SEQUENCE LISTING (191 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 133 aa, Artificial
  Human IL-2 S125I variant sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 5 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 6 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 226 aa, Artificial
  Human IgG1-Fc with reduced/abolished effector function
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 226 aa, Artificial
  Human IgG1-Fc with reduced/abolished effector function and extended half-life
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 9 - protein, 226 aa, Artificial
  Human IgG Knob-Fc with reduced/abolished effector function
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 226 aa, Artificial
  Human IgG Hole-Fc with reduced/abolished effector function
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 11 - protein, 12 aa, Artificial
  Peptide linker
GGGSGGGSGGGS

SEQ ID NO: 12 - protein, 4 aa, Artificial
  Peptide linker
GGGS

SEQ ID NO: 13 - protein, 13 aa, Artificial
  Peptide linker
GSSGGSGGSGGSG

SEQ ID NO: 14 - protein, 5 aa, Artificial
  Peptide linker
GSSGT

SEQ ID NO: 15 - protein, 15 aa, Artificial
  Peptide linker
GGGGSGGGGSGGGGS

SEQ ID NO: 16 - protein, 17 aa, Artificial
  Peptide linker
AEAAAKEAAAKEAAAKA

SEQ ID NO: 17 - protein, 20 aa, Artificial
  Peptide linker
GGGGSGGGGSGGGGSGGGGS

SEQ ID NO: 18 - protein, 8 aa, Artificial
  Peptide linker
GGGSGGGS

SEQ ID NO: 19 - protein, 5 aa, Artificial
  Peptide linker
GSGST

SEQ ID NO: 20 - protein, 4 aa, Artificial
  Peptide linker
GGSS

SEQ ID NO: 21 - protein, 5 aa, Artificial
  Peptide linker
GGGGS

SEQ ID NO: 22 - protein, 4 aa, Artificial
  Peptide linker
GGSG

SEQ ID NO: 23 - protein, 4 aa, Artificial
  Peptide linker
SGGG

SEQ ID NO: 24 - protein, 4 aa, Artificial
  Peptide linker
GSGS

SEQ ID NO: 25 - protein, 6 aa, Artificial
  Peptide linker
GSGSGS

SEQ ID NO: 26 - protein, 8 aa, Artificial
  Peptide linker
GSGSGSGS

SEQ ID NO: 27 - protein, 10 aa, Artificial
  Peptide linker
GSGSGSGSGS

SEQ ID NO: 28 - protein, 12 aa, Artificial
  Peptide linker
GSGSGSGSGSGS

SEQ ID NO: 29 - protein, 10 aa, Artificial
  Peptide linker
GGGGSGGGGS

SEQ ID NO: 30 - protein, 15 aa, Artificial
  Peptide linker
GSGSGSGSGSGSGGS

SEQ ID NO: 31 - protein, 133 aa, Artificial
  IL-2 F42A/S125I variant sequence
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 32 - protein, 133 aa, Artificial; identical to SEQ ID NO: 4
  IL-2 R38F/S125I variant sequence

SEQ ID NO: 33 - protein, 133 aa, Artificial
  IL-2 R38G/S125I variant sequence
APTSSSTKKTQLQLEHLLLDLQMILNGINNYKNPKLTGMLTAKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 34 - protein, 133 aa, Artificial
  IL-2 R38A/S125I variant sequence
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 35 - protein, 133 aa, Artificial
  IL-2 T41A/S125I variant sequence
APTSSSTKKTQLQLEHLLLDLQMILNGINNYKNPKLTRMLAFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 36 - protein, 133 aa, Artificial
  APTSSSTKKTQLQLEHLLLDLQMILNGINNYKNPKLTRMLGFKFYMP [sequence omitted] IVEFLNRWITFIQSIISTLT
APTSSSTKKTQLQLEHLLLDLQMILNGINNYKNPKLTRMLGFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 37 - protein, 133 aa, Artificial
  IL-2 T41V/S125I variant sequence
APTSSSTKKTQLQLEHLLLDLQMILNGINNYKNPKLTRMLVFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 38 - protein, 133 aa, Artificial
  IL-2 F44G/S125I variant sequence
APTSSSTKKTQLQLEHLLLDLQMILNGINNYKNPKLTRMLTFKGYMPKKATELKHLQCLEEELKPLEEVLNLAQSKNFHLRPRDLISNINVIVLELKGSETTFMCEYADETATIVEFLNRWITFIQSIISTLT

SEQ ID NO: 39 - protein, 133 aa, Artificial
  IL-2 F44V/S125I variant sequence
APTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEYADETATIVEFLNRWITFIQSIISTLT

SEQ ID NO: 40 - protein, 133 aa, Artificial
  IL-2 E62A/S125I variant sequence
APTSSSTKKTQLQLEHLLLDLQMILNGINNYKNPKLTRMLTFKFYMPKKATELKHLQCLEEALKPLEEVLNLAQSKNFHLRPRDLISNINVIVLELKGSETTFMCEYADETATIVEFLNRWITFIQSIISTLT

SEQ ID NO: 41 - protein, 133 aa, Artificial
  IL-2 E62F/S125I variant sequence
APTSSSTKKTQLQLEHLLLDLQMILNGINNYKNPKLTRMLTFKFYMPKKATELKHLQCLEEFLKPLEEVLNLAQSKNFHLRPRDLISNINVIVLELKGSETTFMCEYADETATIVEFLNRWITFIQSIISTLT

SEQ ID NO: 42 - protein, 133 aa, Artificial
  IL-2 E62H/S125I variant sequence
APTSSSTKKTQLQLEHLLLDLQMILNGINNYKNPKLTRMLTFKFYMPKKATELKHLQCLEEHLKPLEEVLNLAQSKNFHLRPRDLISNINVIVLELKGSETTFMCEYADETATIVEFLNRWITFIQSIISTLT

SEQ ID NO: 43 - protein, 133 aa, Artificial
  IL-2 E62L/S125I variant sequence
APTSSSTKKTQLQLEHLLLDLQMILNGINNYKNPKLTRMLTFKFYMPKKATELKHLQCLEELLKPLEEVLNLAQSKNFHLRPRDLISNINVIVLELKGSETTFMCEYADETATIVEFLNRWITFIQSIISTLT

SEQ ID NO: 44 - protein, 133 aa, Artificial
  IL-2 P65G/S125I variant sequence
APTSSSTKKTQLQLEHLLLDLQMILNGINNYKNPKLTRMLTFKFYMPKKATELKHLQCLEEELKGLEEVLNLAQSKNFHLRPRDLISNINVIVLELKGSETTFMCEYADETATIVEFLNRWITFIQSIISTLT

SEQ ID NO: 45 - protein, 133 aa, Artificial
  IL-2 P65E/S125I variant sequence
APTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFIQSIISTLT

SEQ ID NO: 46 - protein, 133 aa, Artificial
  IL-2 P65H/S125I variant sequence
APTSSSTKKTQLQLEHLLLDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 47 - protein, 133 aa, Artificial
  IL-2 P65R/S125I variant sequence
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 48 - protein, 133 aa, Artificial
  IL-2 P65A/S125I variant sequence
APTSSSTKKTQLQLEHLLLDLQMILNGINNYKNPKLTRMLTFKFYMPKKATELKHLQCLEEELKALEEVLNLAQSKNFHLRPRDLISNINVIVLELKGSETTFMCEYADETATIVEFLNRWITFIQSIISTLT

SEQ ID NO: 49 - protein, 133 aa, Artificial
  IL-2 P65K/S125I variant sequence
APTSSSTKKTQLQLEHLLLDLQMILNGINNYKNPKLTRMLTFKFYMPKKATELKHLQCLEEELKKLEEVLNLAQSKNFHLRPRDLISNINVIVLELKGSETTFMCEYADETATIVEFLNRWITFIQSIISTLT

SEQ ID NO: 50 - protein, 133 aa, Artificial
  IL-2 P65N/S125I variant sequence
APTSSSTKKTQLQLEHLLLDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 51 - protein, 133 aa, Artificial
  IL-2 P65Q/S125I variant sequence
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 52 - protein, 133 aa, Artificial
  IL-2 E68A/S125I variant sequence
APTSSSTKKTQLQLEHLLLDLQMILNGINNYKNPKLTRMLTFKFYMPKKATELKHLQCLEEELKPLEAVLNLAQSKNFHLRPRDLISNINVIVLELKGSETTFMCEYADETATIVEFLNRWITFIQSIISTLT

SEQ ID NO: 53 - protein, 133 aa, Artificial
  IL-2 E68F/S125I variant sequence
APTSSSTKKTQLQLEHLLLDLQMILNGINNYKNPKLTRMLTFKFYMPKKATELKHLQCLEEELKPLEFVLNLAQSKNFHLRPRDLISNINVIVLELKGSETTFMCEYADETATIVEFLNRWITFIQSIISTLT

SEQ ID NO: 54 - protein, 133 aa, Artificial
  IL-2 E68H/S125I variant sequence
APTSSSTKKTQLQLEHLLLDLQMILNGINNYKNPKLTRMLTFKFYMPKKATELKHLQCLEEELKPLEHVLNLAQSKNFHLRPRDLISNINVIVLELKGSETTFMCEYADETATIVEFLNRWITFIQSIISTLT

SEQ ID NO: 55 - protein, 133 aa, Artificial
  IL-2 E68L/S125I variant sequence
APTSSSTKKTQLQLEHLLLDLQMILNGINNYKNPKLTRMLTFKFYMPKKATELKHLQCLEEELKPLELVLNLAQSKNFHLRPRDLISNINVIVLELKGSETTFMCEYADETATIVEFLNRWITFIQSIISTLT

SEQ ID NO: 56 - protein, 133 aa, Artificial
  IL-2 E68P/S125I variant sequence
APTSSSTKKTQLQLEHLLLDLQMILNGINNYKNPKLTRMLTFKFYMPKKATELKHLQCLEEELKPLEPVLNLAQSKNFHLRPRDLISNINVIVLELKGSETTFMCEYADETATIVEFLNRWITFIQSIISTLT

SEQ ID NO: 57 - protein, 133 aa, Artificial
  IL-2 Y107G/S125I variant sequence
APTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEGADETATIVEFLNRWITFIQSIISTLT

SEQ ID NO: 58 - protein, 133 aa, Artificial
  IL-2 Y107H/S125I variant sequence
APTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEHADETATIVEFLNRWITFIQSIISTLT

SEQ ID NO: 59 - protein, 133 aa, Artificial
  IL-2 Y107L/S125I variant sequence
APTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCELADETATIVEFLNRWITFIQSIISTLT

SEQ ID NO: 60 - protein, 133 aa, Artificial
  IL-2 Y107V/S125I variant sequence
APTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEVADETATIVEFLNRWITFIQSIISTLT

SEQ ID NO: 61 - protein, 133 aa, Artificial
  IL-2 F42A/E62F/S125I variant sequence
APTSSSTKKTQLQLEHLLLDLQMILNGINNYKNPKLTRMLTAKFYMPKKATELKHLQCLEEFLKPLEEVLNLAQSKNFHLRPRDLISNINVIVLELKGSETTFMCEYADETATIVEFLNRWITFIQSIISTLT

SEQ ID NO: 62 - protein, 133 aa, Artificial
  IL-2 F42A/E62A/S125I variant sequence
APTSSSTKKTQLQLEHLLLDLQMILNGINNYKNPKLTRMLTAKFYMPKKATELKHLQCLEEALKPLEEVLNLAQSKNFHLRPRDLISNINVIVLELKGSETTFMCEYADETATIVEFLNRWITFIQSIISTLT

SEQ ID NO: 63 - protein, 133 aa, Artificial
  IL-2 F42A/E62H/S125I variant sequence
APTSSSTKKTQLQLEHLLLDLQMILNGINNYKNPKLTRMLTAKFYMPKKATELKHLQCLEEHLKPLEEVLNLAQSKNFHLRPRDLISNINVIVLELKGSETTFMCEYADETATIVEFLNRWITFIQSIISTLT

SEQ ID NO: 64 - protein, 133 aa, Artificial
  IL-2 F42A/P65H/S125I variant sequence
APTSSSTKKTQLQLEHLLLDLQMILNGINNYKNPKLTRMLTAKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 65 - protein, 133 aa, Artificial
  IL-2 F42A/P65R/S125I variant sequence
APTSSSTKKTQLQLEHLLLDLQMILNGINNYKNPKLTRMLTAKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 66 - protein, 133 aa, Artificial
  IL-2 F42A/P65A/S125I variant sequence
APTSSSTKKTQLQLEHLLLDLQMILNGINNYKNPKLTRMLTAKFYMPKKATELKHLQCLEEELKALEEVLNLAQSKNFHLRPRDLISNINVIVLELKGSETTFMCEYADETATIVEFLNRWITFIQSIISTLT

SEQ ID NO: 67 - protein, 367 aa, Artificial
  P-0250
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 68 - protein, 367 aa, Artificial
  P-0531
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 69 - protein, 367 aa, Artificial
  P-0613
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 70 - protein, 367 aa, Artificial
  P-0614
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTFMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 71 - protein, 367 aa, Artificial
  P-0615
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTGMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 72 - protein, 367 aa, Artificial
  P-0602
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 73 - protein, 367 aa, Artificial
  P-0603
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLAFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 74 - protein, 367 aa, Artificial
  P-0604
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLGFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 75 - protein, 367 aa, Artificial
  P-0605
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLVFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 76 - protein, 367 aa, Artificial
  P-0606
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKGYMPKKATELKHLQCLEEELKPLEEVLNLAQSKNFHLRPRDLISNINVIVLELKGSETTFMCEYADETATIVEFLNRWITFIQSIISTLT

SEQ ID NO: 77 - protein, 367 aa, Artificial
  P-0607
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEYADETATIVEFLNRWITFIQSIISTLT

SEQ ID NO: 78 - protein, 367 aa, Artificial
  P-0624
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEALKPLEEVLNLAQSKNFHLRPRDLISNINVIVLELKGSETTFMCEYADETATIVEFLNRWITFIQSIISTLT

SEQ ID NO: 79 - protein, 367 aa, Artificial
  P-0625
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEFLKPLEEVLNLAQSKNFHLRPRDLISNINVIVLELKGSETTFMCEYADETATIVEFLNRWITFIQSIISTLT

SEQ ID NO: 80 - protein, 367 aa, Artificial
  P-0626
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEHLKPLEEVLNLAQSKNFHLRPRDLISNINVIVLELKGSETTFMCEYADETATIVEFLNRWITFIQSIISTLT

SEQ ID NO: 81 - protein, 367 aa, Artificial
  P-0627
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEELLKPLEEVLNLAQSKNFHLRPRDLISNINVIVLELKGSETTFMCEYADETATIVEFLNRWITFIQSIISTLT

SEQ ID NO: 82 - protein, 367 aa, Artificial
  P-0608
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKGLEEVLNLAQSKNFHLRPRDLISNINVIVLELKGSETTFMCEYADETATIVEFLNRWITFIQSIISTLT

SEQ ID NO: 83 - protein, 367 aa, Artificial
  P-0633
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFIQSIISTLT

SEQ ID NO: 84 - protein, 367 aa, Artificial
  P-0634
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 85 - protein, 367 aa, Artificial
  P-0635
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 86 - protein, 367 aa, Artificial
  P-0628
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEAVLNLAQSKNFHLRPRDLISNINVIVLELKGSETTFMCEYADETATIVEFLNRWITFIQSIISTLT

SEQ ID NO: 87 - protein, 367 aa, Artificial
  P-0629
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEFVLNLAQSKNFHLRPRDLISNINVIVLELKGSETTFMCEYADETATIVEFLNRWITFIQSIISTLT

SEQ ID NO: 88 - protein, 367 aa, Artificial
  P-0630
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEHVLNLAQSKNFHLRPRDLISNINVIVLELKGSETTFMCEYADETATIVEFLNRWITFIQSIISTLT

SEQ ID NO: 89 - protein, 367 aa, Artificial
  P-0631
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLELVLNLAQSKNFHLRPRDLISNINVIVLELKGSETTFMCEYADETATIVEFLNRWITFIQSIISTLT

SEQ ID NO: 90 - protein, 367 aa, Artificial
  P-0632
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEPVLNLAQSKNFHLRPRDLISNINVIVLELKGSETTFMCEYADETATIVEFLNRWITFIQSIISTLT

SEQ ID NO: 91 - protein, 367 aa, Artificial
  P-0609
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEGADETATIVEFLNRWITFIQSIISTLT

SEQ ID NO: 92 - protein, 367 aa, Artificial
  P-0610
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEHADETATIVEFLNRWITFIQSIISTLT

SEQ ID NO: 93 - protein, 367 aa, Artificial
  P-0611
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCELADETATIVEFLNRWITFIQSIISTLT

SEQ ID NO: 94 - protein, 367 aa, Artificial
  P-0612
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKVYMPKKATELKHLQCLEEELKPLEEVLNLAQSKNFHLRPRDLISNINVIVLELKGSETTFMCEVADETATIVEFLNRWITFIQSIISTLT

SEQ ID NO: 95 - protein, 367 aa, Artificial
  P-0551
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFIQSIISTLT

SEQ ID NO: 96 - protein, 367 aa, Artificial
  P-0704 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 97 - protein, 367 aa, Artificial
  P-0706 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKALEEVLNLAQSKNFHLRPRDLISNINVIVLELKGSETTFMCEYADETATIVEFLNRWITFIQSIISTLT

SEQ ID NO: 98 - protein, 367 aa, Artificial
  P-0707 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKKLEEVLNLAQSKNFHLRPRDLISNINVIVLELKGSETTFMCEYADETATIVEFLNRWITFIQSIISTLT

SEQ ID NO: 99 - protein, 367 aa, Artificial
  P-0708 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 100 - protein, 367 aa, Artificial
  P-0709 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 101 - protein, 367 aa, Artificial
  P-0702 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEFLKPLEEVLNLAQSKNFHLRPRDLISNINVIVLELKGSETTFMCEYADETATIVEFLNRWITFIQSIISTLT

SEQ ID NO: 102 - protein, 367 aa, Artificial
  P-0766 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEALKPLEEVLNLAQSKNFHLRPRDLISNINVIVLELKGSETTFMCEYADETATIVEFLNRWITFIQSIISTLT

SEQ ID NO: 103 - protein, 367 aa, Artificial
  P-0767 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEHLKPLEEVLNLAQSKNFHLRPRDLISNINVIVLELKGSETTFMCEYADETATIVEFLNRWITFIQSIISTLT

SEQ ID NO: 104 - protein, 367 aa, Artificial
  P-0703 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 105 - protein, 367 aa, Artificial
  P-0705 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 106 - protein, 367 aa, Artificial
  P-0765 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFYMPKKATELKHLQCLEEELKALEEVLNLAQSKNFHLRPRDLISNINVIVLELKGSETTFMCEYADETATIVEFLNRWITFIQSIISTLT

SEQ ID NO: 107 - protein, 367 aa, Artificial
  P-0689 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 108 - protein, 367 aa, Artificial
  Benchmark knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFIQSIISTLT

SEQ ID NO: 109 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 110 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 111 - protein, 133 aa, Artificial
  IL-2 L19H/P65R/S125I variant sequence
APTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 112 - protein, 133 aa, Artificial
  IL-2 L19Q/P65R/S125I variant sequence
APTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 113 - protein, 133 aa, Artificial
  IL-2 L19Y/P65R/S125I variant sequence
APTSSSTKKTQLQLEHLLYDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 114 - protein, 133 aa, Artificial
  IL-2 L19H/P65Q/S125I variant sequence
APTSSSTKKTQLQLEHLLHDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 115 - protein, 133 aa, Artificial
  IL-2 L19H/P65H/S125I variant sequence
APTSSSTKKTQLQLEHLLHDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 116 - protein, 133 aa, Artificial
  IL-2 L19H/P65N/S125I variant sequence
APTSSSTKKTQLQLEHLLHDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 117 - protein, 133 aa, Artificial
  IL-2 L19Q/P65Q/S125I variant sequence
APTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 118 - protein, 133 aa, Artificial
  IL-2 L19Q/P65H/S125I variant sequence
APTSSSTKKTQLQLEHLLQDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 119 - protein, 133 aa, Artificial
  IL-2 L19Q/P65N/S125I variant sequence
APTSSSTKKTQLQLEHLLQDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 120 - protein, 133 aa, Artificial
  IL-2 P65R/S125I/Q126E variant sequence
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIESIISTLT

SEQ ID NO: 121 - protein, 367 aa, Artificial
  P-0731 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 122 - protein, 367 aa, Artificial
  P-0759 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 123 - protein, 367 aa, Artificial
  P-0761 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLYDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 124 - protein, 367 aa, Artificial
  P-0811 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLHDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 125 - protein, 367 aa, Artificial
  P-0812 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLHDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 126 - protein, 367 aa, Artificial
  P-0813 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLHDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 127 - protein, 367 aa, Artificial
  P-0814 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 128 - protein, 367 aa, Artificial
  P-0815 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 129 - protein, 367 aa, Artificial
  P-0816 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 130 - protein, 367 aa, Artificial
  P-0732 knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIESIISTLT

SEQ ID NO: 131 - protein, 367 aa, Artificial
  P-0758
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 132 - protein, 367 aa, Artificial
  P-0760
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 133 - protein, 367 aa, Artificial
  P-0762
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLYDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 134 - protein, 226 aa, Artificial
  Knob-Fc with extended in vivo half-life
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHAHYTQKSLSLSPG

SEQ ID NO: 135 - protein, 226 aa, Artificial
  Hole-Fc with extended in vivo half-life
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHAHYTQKSLSLSPG

SEQ ID NO: 136 - protein, 449 aa, Artificial
  Humanized anti-FAP antibody heavy chain
QVQLVQSGAEVKKPGASVKVSCKASGYTFTENIIHWVRQAPGQGLEWMGWFHPGSGSIKYAQKFQGRVTMTADKSTSTVYMELSSLRSEDTAVYYCARHGGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 218 aa, Artificial
  Humanized anti-FAP antibody kappa light chain
DIQMTQSPSSLSASVGDRVTITCRASRSISTSAYSYMHWYQQKPGKAPKLLIYLASNLESGVPSRFSGSGSGTDFTLTISSLQPEDFATYYCQHSRELPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 448 aa, Artificial
  Human PD-1 antagonist antibody heavy chain
EVQLVQSGAEVKKPGASVKVSCKASGYRFTSYGISWVRQAPGQGLEWMGWISAYNGNTNYAQKLQGRVTMTTDTSTNTAYMELRSLRSDDTAVYYCARDADYSSGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 214 aa, Artificial
  Human PD-1 antagonist antibody L?
SYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 140 - protein, 446 aa, Artificial
  Humanized PD-1 antagonist antibody-HC
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 214 aa, Artificial
  Humanized PD-1 antagonist antibody-L
DIVMTQSPLSLPVTPGEPASITCKASQDVETVVAWYLQKPGQSPRLLIYWASTRHTGVPDRFSGSGSGTDFTLKISRVEAEDVGVYYCQQYSRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 452 aa, Artificial
  Humanized PD-1 antagonist antibody-HC
QGQLVQSGAEVKKPGASVKVSCKASGYTFTDYEMHWVRQAPGQGLEWMGVIESETGGTAYNQKFKGRAKITADKSTSTAYMELSSLRSEDTAVYYCTREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 219 aa, Artificial
  Humanized PD-1 antagonist antibody-L
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 447 aa, Artificial
  Humanized PD-1 antagonist antibody-HC
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - protein, 218 aa, Artificial
  Humanized PD-1 antagonist antibody-L
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 440 aa, Artificial
  Human PD-1 antagonist antibody-HC
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 214 aa, Artificial
  Human PD-1 antagonist antibody-L
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 448 aa, Artificial
  Humanized PD-L1 antagonist antibody-HC
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 214 aa, Artificial
  Humanized PD-L1 antagonist antibody-L
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 448 aa, Artificial
  Human CTLA-4 antagonist antibody-HC
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 215 aa, Artificial
  Human CTLA-4 antagonist antibody-L
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 452 aa, Artificial
  Human CD40 agonist antibody-HC
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 153 - protein, 214 aa, Artificial
  Human CD40 agonist antibody-L
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 445 aa, Artificial
  Humanized anti-fibronectin antibody-HC
EVQLLESGGGLVQPGGSLRLSCAASGFTFSSFSMSWVRQAPGKGLEWVSSISGSSGTTYYADSVKGRFTISRDSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 215 aa, Artificial
  Humanized anti-fibronectin antibody-L
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 451 aa, Artificial
  Chimeric anti-CD20 antibody-HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 213 aa, Artificial
  Chimeric anti-CD20 antibody-L
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 450 aa, Artificial
  Humanized anti-Her2 antibody-HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 214 aa, Artificial
  Humanized anti-Her2 antibody-L
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 449 aa, Artificial
  Chimeric anti-EGFR antibody-HC
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 214 aa, Artificial
  Chimeric anti-EGFR antibody-L
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 329 aa, Artificial
  Human IgG1 CH1-CH2-CH3 domain with reduced/abolished effector function
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 329 aa, Artificial
  Human IgG1 CH1-CH2-CH3 domain with reduced/abolished effector function knob chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 329 aa, Artificial
  Human IgG1 CH1-CH2-CH3 domain with reduced/abolished effector function Hole chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 165 - protein, 587 aa, Artificial
  Humanized PD-1 antagonist antibody -HC-IL-2 variant
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGGGGSGGGSAPTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 166 - protein, 587 aa, Artificial
  Humanized PD-1 antagonist antibody -HC-IL-2 variant  knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 167 - protein, 587 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 168 - protein, 587 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 169 - protein, 446 aa, Artificial
  Humanized PD-1 antagonist antibody-IgG1-HC hole chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 170 - protein, 165 aa, Artificial
  Human IL-2R? Sushi domains sequence
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 171 - protein, 554 aa, Artificial
  P-0327
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 172 - protein, 548 aa, Artificial
  P-0422
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGGGGSGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 367 aa, Artificial
  P-0482-Hole chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 174 - protein, 405 aa, Artificial
  P-0482-Knob chain
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICT

SEQ ID NO: 175 - protein, 446 aa, Artificial
  Humanized PD-1 antagonist antibody-IgG1-HC hole chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 176 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 177 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 178 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 179 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 180 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLHDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 181 - protein, 594 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLQDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 182 - protein, 449 aa, Artificial
  Benchmark PD-1 antagonist antibody-HC-hole chain
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 183 - protein, 597 aa, Artificial
  Benchmark PD-1 antagonist antibody-HC-Benchmark IL-2 variant knob chain
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 184 - protein, 449 aa, Artificial; identical to SEQ ID NO: 182
  Benchmark PD-1 antagonist antibody-L?

SEQ ID NO: 185 - protein, 443 aa, Artificial
  Surrogate mouse PD-1 antagonist antibody HC chain 1
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 186 - protein, 443 aa, Artificial
  Surrogate mouse PD-1 antagonist antibody HC chain 2
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 187 - protein, 218 aa, Artificial
  Surrogate mouse PD-1 antagonist antibody LC
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 188 - protein, 133 aa, Artificial
  Benchmark IL-2 variant
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 189 - protein, 586 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 190 - protein, 585 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 191 - protein, 584 aa, Artificial
  Humanized PD-1 antagonist antibody-HC-IL-2 variant knob chain
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

(19) United States
(12) Patent Application Publication
(10) Pub. No.: US 2022/0235109 A1
(43) Pub. Date: Jul. 28, 2022

(54) NOVEL INTERLEUKIN-2 VARIANTS FOR THE TREATMENT OF CANCER

(71) Applicant: Cugene Inc, Waltham, MA (US)

(72) Inventors: Yue-Sheng Li, Thousand Oaks, CA (US); Lingyun Rui, Weston, MA (US); Jing Xu, Waltham, MA (US)

(21) Appl. No.: 17/618,140

(22) PCT Filed: Jun. 13, 2020
(86) PCT No.: PCT/US20/37644

§ 371 Date: Dec. 10, 2021

Related U.S. Application Data
(60) Provisional application No. 62/947,806, filed on Dec. 13, 2019.
(60) Provisional application No. 62/861,651, filed on Jun. 14, 2019.

(51) Int. Cl. C07K 14/55; C07K 16/28

(52) U.S. Cl. CPC C07K 14/55; C07K 16/2818; C07K 2317/24; C07K 2317/92; C07K 2317/76

(57) ABSTRACT

The present invention relates to polypeptides which share primary sequence with human IL-2, except for several amino acids that have been mutated. A panel of IL-2 variants comprise mutations substantially reduce the ability of these polypeptides to stimulate Treg cells and make them more effective in the therapy of tumors. Also includes therapeutic uses of these mutated variants, used alone or in combination with vaccines, or TAA-targeting biologics, or immune checkpoint blocker, or as the building block in bifunctional molecule construct, for the therapy of diseases such as cancer or infections where the activity of regulatory T cells (Tregs) is undesirable. In another aspect the present invention relates to pharmaceutical compositions comprising the polypeptides disclosed. Finally, the present invention relates to the therapeutic use of the polypeptides and pharmaceutical compositions disclosed due to their selective modulating effect of the immune system on diseases like autoimmune and inflammatory disorders, cancer, and various infectious diseases.

### RELATED PATENT APPLICATIONS

This application claims benefit of U.S. Provisional Application No. 62/947,806, filed on Dec. 13, 2019, and U.S. Provisional Application No. 62/861,651, filed on Jun. 14, 2019, each incorporated in its entirety by reference herein.

### BACKGROUND ART

Interleukin 2 (IL-2) was the first growth factor described for T cells. Since its discovery it has been shown to promote proliferation and survival of T cells in vitro (Smith, K A. (1988) Science. 240, 1169-76) and the ability to boost immune response in the context of T viral infections (Blattman, J N, et al. (2003) Nat Med 9, 540-7) or vaccines (Fishman, M., et al. (2008) J Immunother. 31, 72-80, Kudo-Saito, C., et al. (2007) Cancer Immunol Immunother. 56, 1897-910; Lin, C T., et al. (2007) Immunol Lett. 114, 86-93).

IL-2 has been used in cancer therapy. Recombinant human IL-2 is an effective immunotherapy for metastatic melanoma and renal cancer, with durable responses in approximately 10% of patients. However short half-life and severe toxicity limits the optimal dosing of IL-2. Further, IL-2 also binds to its heterotrimeric receptor IL-2Rαβγ with greater affinity, which preferentially expands immunosuppressive regulatory T cells (Tregs) expressing high constitutive levels of IL-2Rα. Expansion of Tregs represents an undesirable effect of IL-2 for cancer immunotherapy. Consequently, successful immunotherapy of cancers using IL-2 has to address two fundamentally important issues: 1) how to limit side effects yet be active where it is needed; and 2) how to preferentially activate effector T cells while limiting the stimulation of Tregs.

More recently, it was found that IL-2 could be modified to selectively stimulate cytotoxic effector T cells. Various approaches have led to the generation of IL-2 variants with improved and selective immune stimulatory capacities. Some of these IL-2 variants were designed to increase the capacity of this molecule to signal mainly by the high affinity receptor (alpha, beta and gamma chains) and not by the intermediate affinity receptor (beta and gamma chains). The basic idea was to promote signaling in T cells instead of signaling in NK cells, which were believed to be responsible for the observed toxic effects. The following inventions are in this line of work: U.S. Pat. Nos. 7,186,804, 7,105,653, 6,955,807, 5,229,109, U.S. Patent Application 20050142106. It is important to note that none of these inventions relates to variants of IL-2 that have greater therapeutic efficacy than the native IL-2 in vivo.

In summary, IL-2 is a highly pleiotropic cytokine which is very relevant in the biological activity of different cell populations. This property makes the IL-2 an important node in the regulation of the immune response, making it an attractive target for therapies and complex immune modulation. Further, receptor subunit-biased IL-2 variants can be made to achieve IL-2 mediated selective immune modulation to preferentially expand and activate Teff cells to attack cancer cells while reducing Treg cell expansion and activation.

### DISCLOSURE OF THE INVENTION

In one aspect, the present invention relates to the production of mutated variants of IL-2, which are characterized by being selective agonists of IL-2 activity with reduced or abolished binding capability to IL-2Rα. Specifically, these variants will provide a way to overcome the limitations observed in native IL-2 therapy which are derived from their proven ability to expand in vivo natural regulatory T cells. The present invention relates to polypeptides which share their primary sequence with the human IL-2, except for several amino acids that have been mutated. The mutations introduced substantially reduce the ability of these polypeptides to stimulate Treg cells and give IL-2 a greater efficacy. In addition, the mutations introduced are expected to decrease CD25-mediated VLS and CD25-mediated sink effect. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections where the activity of regulatory T cells (Tregs) is undesirable.

In one aspect, the present invention relates to the production of mutated variants of IL-2, which are characterized by being selective agonists of IL-2 activity with optimally modulated overall potency by reducing IL-2Rβγ interaction in addition to reduced or abolished binding capability to IL-2Rα. The mutations introduced prevent over-activation of the pathway, reduce undesirable “on-target” “off-tissue” toxicity, decrease potential sink, lower activation induced cell exhaustion associated with lymphocyte overstimulation, mitigate receptor mediated IL-2 internalization, and thus, prolong the in vivo half-life and result in slow and durable pharmacodynamics to improve biodistribution, bioavailability, function, and anti-tumor efficacy. The present inventors also propose that the use of IL-2 variants with reduced/abolished binding to IL-2Rα and attenuated IL-2Rβγ activity is to facilitate the establishment of stoichiometric balance between the cytokine and antibody arms exhibiting dramatically different potency and molecular weights to allow optimal dosing and maintain function of each arm. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections

In one aspect, the present invention relates to the production of mutated variants of IL-2, which are characterized by being selective agonists of IL-2 activity with reduced IL-2Rβγ interaction in addition to reduced or abolished binding capability to IL-2Rα. The mutations introduced provide prolonged and durable pharmacodynamics and potentially pharmacokinetics. In addition, the mutations introduced reduce cell exhaustion and activation induced cell death and enhance durable lymphocyte responsiveness. As a result, the mutations introduced allow less frequent dosing regimen and offer dosing convenience in clinic. Cost of goods reduction is also expected. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections

In one aspect, the present invention relates to the production of mutated variants of IL-2, which are characterized by being selective agonists of IL-2 activity with abolished binding to IL-2Rα and bolstered effector T and NK cells responses at unexpected high magnitude unmatched by the wild-type counterpart. The CD25-binding abolishing mutations are expected to reduce sink to CD25 or CD25+ cells and consequently increased the availability to IL-2Rβγ. The enriched receptor occupancy elicits vigorous cytotoxic cell response and strong tumor killing efficacy. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections

In one aspect of the current invention, the mutations introduced reduced binding ability to IL-2Rα (CD25) but retained low levels of Treg response. The residue immune regulatory Tregs provide immune counterbalance to improve systemic tolerability and ensure the immune balance not tilted excessively to cytotoxic effector cells. The fined-tuned Treg response is situated not to suffer tumor killing efficacy but strong enough to maintain peripheral tolerance. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections

In one aspect, the present invention relates to the production of mutated variants of IL-2, which possess reduced aggregation, increased expression, improved manufacturability and developability with a combination of attributes including, for example, substantially reduced ability to stimulate Treg cells, reduced receptor over-activation, reduced undesirable “on-target” “off-tissue” toxicity, and prolonged pharmacodynamics to improve biodistribution, bioavailability, function, and anti-tumor efficacy. The present invention relates to polypeptides which share their primary sequence with the human IL-2, except for one to several amino acids that have been mutated. The present invention also includes therapeutic uses of these mutated variants, alone or in combination with vaccines, or immune checkpoint inhibitors, or tumor associated antigen (TAA)-targeting biologics, or as part of the bifunctional fusion construct for therapy of diseases such as cancer or infections

In one aspect, the present invention relates to the production of mutated variants of IL-2, which are characterized by the reduction of severe toxicity, such as vascular leak syndrome (VLS), associated with high dose IL-2 in clinical for treatment of renal carcinoma and melanoma. Specifically, the mutations introduced substantially reduce binding ability to IL-2Rα (CD25); consequently, impair binding to CD25+ pulmonary endothelial cells, and is expected to prevent endothelial cell damage and significantly reduce VLS. The present invention relates to polypeptides which share their primary sequence with the human IL-2, except for one to several amino acids that have been mutated. The present invention also includes therapeutic uses of these mutated variants, alone or in combination with vaccines, or immune checkpoint modulators, or tumor associated antigen (TAA)-targeting biologics, or as part of the bifunctional fusion construct for therapy of diseases such as cancer or infections to improve safety profile.

The present invention allows for a substantial improvement of the current strategies of immunomodulation based on IL-2 in the therapy of cancer. Specifically, the replacement of the native IL-2 by the mutated variants described herein, will result in no preferential stimulation of Treg cells over cytotoxic effector cells, reduction of undesirable “on-target” “off-tissue” toxicity, minimization of overstimulation associated cell exhaustion, and improvement of pharmacodynamics and potentially pharmacokinetics. Mutations are expected to impair binding to CD25+ pulmonary endothelial cells and consequently reduce VLS. In various embodiments, the IL-2 variant (or mutant) comprises the sequence of the IL-2 variant (or mutant) derived from the sequence of the mature human IL-2 polypeptide as set forth in SEQ ID NO: 3. In various embodiments, the IL-2 variant functions as an IL-2 agonist. In various embodiments, the IL-2 variant functions as an IL-2 antagonist. In various embodiments, the IL-2 variants comprise SEQ ID NOS: 31-66, or SEQ ID NOS: 111-120 or amino acids 9-133, 10-133, and 11-113 of SEQ ID NO: 47.

In another aspect, the IL-2 variants of the present invention are attached to at least one heterologous protein. In various embodiments, IL-2 variants are fused to at least one polypeptide that confers extended half-life on the fusion molecule. Such polypeptides include an IgG Fc or other polypeptides that bind to the neonatal Fc receptor, human serum albumin, or polypeptides that bind to a protein having extended serum half-life. In various embodiments, the IL-2 variant is fused to an IgG Fc molecule. In various embodiments, the Fc domain is a human IgG Fc domain. In various embodiments, the Fc domain is derived from the human IgG1 heavy chain constant domain sequence set forth in SEQ ID NO: 6. In various embodiments, the Fc domain is an Fc domain having the amino acid sequence set forth in SEQ ID NO: 7. In various embodiments, the Fc domain is an Fc domain having the amino acid sequence set forth in SEQ ID NO: 8. In various embodiments, the Fc domain is derived from the human IgG2 heavy chain constant domain sequence. In various embodiments, the Fc domain is derived from the human IgG4 heavy chain constant domain sequence.

In various embodiments, the IL-2 variants can be linked to the N-terminus or the C-terminus of the IgG Fc region.

The term “Fc” refers to molecule or sequence comprising the sequence of a non-antigen-binding fragment of whole antibody, whether in monomeric or multimeric form. The original immunoglobulin source of the native Fc is preferably of human origin and may be any of the immunoglobulins disclosed in the art. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms. Fc domains containing binding sites for Protein A, Protein G, various Fc receptors and complement proteins.

In various embodiments, the term “Fc variant” refers to a molecule or sequence that is modified from a native Fc but still comprises a binding site for the salvage receptor, FcRn. International applications WO 97/34631 (published Sep. 25, 1997) and WO 96/32458 describe exemplary Fc variants, as well as interaction with the salvage receptor, and are hereby incorporated by reference. Furthermore, a native Fc comprises sites that may be removed because they provide structural features or biological activity that are not required for the fusion molecules of the present invention. Thus, in various embodiments, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by recombinant gene expression or by other means. In various embodiments, an “Fc domain” refers to a dimer of two Fc domain monomers (SEQ ID NO: 6) that generally includes full or part of the hinge region. In various embodiments, an Fc domain may be mutated to lack effector functions. In various embodiments, each of the Fc domain monomers in an Fc domain includes amino acid substitutions in the CH2 antibody constant domain to reduce the interaction or binding between the Fc domain and an Fcγ receptor. In various embodiments, each subunit of the Fc domain comprises three amino acid substitutions that reduce binding to an activating Fc receptor and/or effector function wherein said amino acid substitutions are L234A, L235A and G237A (SEQ ID NO: 7). In various embodiments, each subunit of the Fc domain comprises three amino acid substitutions that reduce binding to an activating Fc receptor and/or effector function wherein said amino acid substitutions are L234A, L235A and P329G.

In various embodiments, an Fc domain may be mutated to further extend in vivo half-life. In various embodiments, each subunit of the Fc domain comprises the three amino acid substitutions M252Y, S254T, and T256E, disclosed in U.S. Pat. No. 7,658,921 that enhance binding to human FcRn. In various embodiments, each subunit of the Fc domain comprises the amino acid substitution N434A (SEQ ID NO: 8) disclosed in U.S. Pat. No. 7,371,826. In various embodiments, each subunit of the Fc domain comprises either the amino acid substitution M428L or N434S, disclosed in U.S. Pat. No. 8,546,543 that enhances binding to human FcRn. In various embodiments, half-life extension mutations can be combined with amino acid substitutions that reduce binding to an activating Fc receptor and/or effector function.

In various embodiments, the IL-2 variant Fc-fusion protein will be monomeric, i.e., contain only a single IL-2 mutein molecule. In such embodiments, the fusion protein is co-expressed with a heterodimeric Fc (e.g. a Knob-Fc having the sequence set forth in SEQ ID NO: 9) linked to an IL-2 variant and the matching heterodimeric Fc (e.g. a Hole-Fc having the sequence set forth in SEQ ID NO: 10). When the heterodimer of the two Fc-containing polypeptides forms, the resulting protein comprises only a monovalent IL-2 variant. In various embodiments, the heterodimeric Fc domain used to make monovalent IL-2 Fc fusion proteins is a Knob Fc domain with reduced/abolished effector function and extended half-life (SEQ ID NO: 134) and a Hole-Fc domain with reduced/abolished effector function and extended half-life (SEQ ID NO: 135).

In various embodiments, the IL-2 variants of the present invention can be attached to an antibody that confers extended half-life on the fusion molecule, such as anti-keyhole limpet hemocyanin (KLH) antibody. Such an antibody recognizes a foreign antigen, confers longer half-life but have no biological function or harm in human. The IgG class could be IgG, IgA, IgE or subclass (e.g., IgG1, IgG2, IgG3, IgA1, IgA2).

In various embodiments, the IL-2 variant constructs of the present invention comprise a targeting moiety in the form of an antibody, an antibody fragment, a protein or a peptide binding to a molecule enriched in the cancer tissue, such as a tumor associated antigen (TAA).

The TAA can be any molecule, macromolecule, combination of molecules, etc. against which an immune response is desired. The TAA can be a protein that comprises more than one polypeptide subunit. For example, the protein can be a dimer, trimer, or higher order multimer. In various embodiments, two or more subunits of the protein can be connected with a covalent bond, such as, for example, a disulfide bond. In various embodiments, the subunits of the protein can be held together with non-covalent interactions. Thus, the TAA can be any peptide, polypeptide, protein, nucleic acid, lipid, carbohydrate, or small organic molecule, or any combination thereof, against which the skilled artisan wishes to induce an immune response. In various embodiments, the TAA is a peptide that comprises about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 400, about 500, about 600, about 700, about 800, about 900 or about 1000 amino acids. In various embodiments, the peptide, polypeptide, or protein is a molecule that is commonly administered to subjects by injection.

In various embodiments, the tumor-specific antibody or binding protein serves as a targeting moiety to guide the IL-2 variant to the diseased site, such as a tumor site, where they can stimulate more optimal anti-tumor immune responses while avoiding the systemic toxicities of free cytokine therapy. For an IL-2 full agonist, IL-2-IL-2R interactions, rather than antibody-antigen targeting, can dictate immunocytokine localization to IL-2 receptor-expressing cells rather than tumor cells at typical antibody doses. In various embodiments, the use of IL-2 variants with reduced/abolished binding to IL-2Rα and attenuated potency in antibody fusion protein facilitates the establishment of stoichiometric balance between the IL-2 and the targeting antibody to achieve optimal dosing at which the antibody can achieve sufficient target occupancy while the IL-2 moiety does not cause over activation of the pathway. The use of IL-2 variants with reduced/abolished binding to IL-2Rα and attenuated potency in the IL-2 antibody fusion proteins and further enhance tumor targeting via the antibody. minimize peripheral activation and AICD, mitigate antigen-sink, and promote tumor targeting via the antibody arm.

In various embodiments, the IL-2 variants of the present invention can be attached to targeting/dual functional moiety that is an antibody, an antibody fragment, a protein, or a peptide targeting immune checkpoint modulators.

A number of immune-checkpoint protein antigens have been reported to be expressed on various immune cells, including, e.g., SIRP (expressed on macrophage, monocytes, dendritic cells), CD47 (highly expressed on tumor cells and other cell types), VISTA (expressed on monocytes, dendritic cells, B cells, T cells), CD152 (expressed by activated CD8+ T cells, CD4+ T cells and regulatory T cells), CD279 (expressed on tumor infiltrating lymphocytes, expressed by activated T cells (both CD4 and CD8), regulatory T cells, activated B cells, activated NK cells, anergic T cells, monocytes, dendritic cells), CD274 (expressed on T cells, B cells, dendritic cells, macrophages, vascular endothelial cells, pancreatic islet cells), and CD223 (expressed by activated T cells, regulatory T cells, anergic T cells, NK cells, NKT cells, and plasmacytoid dendritic cells)(see, e.g., Pardoll, D., Nature Reviews Cancer, 12:252-264, 2012). Antibodies that bind to an antigen which is determined to be an immune-checkpoint protein are known to those skilled in the art. For example, various anti-CD276 antibodies have been described in the art (see, e.g., U.S. Pat. Public. No. 20120294796 (Johnson et al) and references cited therein); various anti-CD272 antibodies have been described in the art (see, e.g., U.S. Pat. Public. No. 20140017255 (Mataraza et al) and references cited therein); various anti-CD152/CTLA-4 antibodies have been described in the art (see, e.g., U.S. Pat. Public. No. 20130136749 (Korman et al) and references cited therein); various anti-LAG-3/CD223 antibodies have been described in the art (see, e.g., U.S. Pat. Public. No. 20110150892 (Thudium et al) and references cited therein); various anti-CD279 (PD-1) antibodies have been described in the art (see, e.g., U.S. Pat. No. 7,488,802 (Collins et al) and references cited therein); various anti-CD274 (PD-L1) antibodies have been described in the art (see, e.g., U.S. Pat. Public. No. 20130122014 (Korman et al) and references cited therein); various anti-TIM-3 antibodies have been described in the art (see, e.g., U.S. Pat. Public. No. 20140044728 (Takayanagi et al) and references cited therein); and various anti-B7-H4 antibodies have been described in the art (see, e.g., U.S. Pat. Public. No. 20110085970 (Terrett et al) and references cited therein); and various anti-TIGIT antibodies have been described in the art (see, e.g., U.S. Pat. Public. No. 20180169239A1 (Grogan) and references cited therein). Each of these references is hereby incorporated by reference in its entirety for the specific antibodies and sequences taught therein.

In various embodiments, IL-2 variant can be fused to an antibody, antibody fragment, or protein or peptide that exhibit binding to an immune-checkpoint protein antigen that is present on the surface of an immune cell. In various embodiments, the immune-checkpoint protein antigen is selected from the group consisting of, but not limited to, CD279 (PD-1), CD274 (PDL-1), CD276, CD272, CD152, CD223 (LAG-3), CD40, SIRPα, CD47, OX-40, GITR, ICOS, CD27, 4-1 BB, TIM-3, B7-H3, B7-H4, TIGIT, and VISTA.

In various embodiments, the antibody is an antagonistic FAP antibody or antibody fragment. In various embodiments, the antibody is a humanized antagonistic FAP antibody comprising the variable domain sequences set forth in SEQ ID NOS: 136 and 137. In various embodiments, the heterologous protein is an antibody or an antibody fragment to an immune checkpoint modulator. In various embodiments, the antibody is an antagonistic PD-1 antibody or antibody fragment. In various embodiments, the antibody is an antagonistic PD-1 antibody comprising the variable domain sequences set forth in SEQ ID NOS: 138 and 139, SEQ ID NOS: 140 and 141, SEQ ID NOS: 142 and 143, SEQ ID NOS: 144 and 145, or SEQ ID NOS: 146 and 147. In various embodiments, the antibody is an antagonistic human PD-L1 antibody comprising the variable domain sequences set forth in SEQ ID NOS: 148 and 149. In various embodiments, the antibody is an antagonistic CTLA-4 antibody comprising the variable domain sequences set forth in SEQ ID NOS: 150 and 151. In various embodiments, the heterologous protein is attached to the IL-2 variant by a linker and/or a hinge linker peptide. The linker or hinge linker may be an artificial sequence of between 5, 10, 15, 20, 30, 40 or more amino acids that are relatively free of secondary structure.

In various embodiments, the heterologous protein is attached to the IL-2 variant by a rigid linker peptide of between 10, 15, 20, 30, 40 or more amino acids that display α-helical conformation and may act as rigid spacers between protein domains.

In another aspect, IL-2 variant can be linked to various nonproteinaceous polymers, including, but not limited to, various polyols such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. In various embodiments, amino acid substitutions may be made in various positions within the IL-2 variants to facilitate the addition of polymers such as PEG. In various embodiments, such PEGylated proteins may have increased half-life and/or reduced immunogenicity over the non-PEGylated proteins.

In various embodiments, IL-2 variants can be linked non-covalently or covalently to an IgG Fc or other polypeptides that bind to the neonatal Fcγ/receptor, human serum albumin, or polypeptides that bind to a protein having extended serum half-life, or various nonproteinaceous polymers at either the N-terminus or C-terminus.

In another aspect, the present disclosure provides a pharmaceutical composition comprising the isolated IL-2 variants in admixture with a pharmaceutically acceptable carrier.

In another aspect, the present disclosure provides a method for treating cancer or cancer metastasis in a subject, comprising administering a therapeutically effective amount of the pharmaceutical compositions of the invention to a subject in need thereof. In one embodiment, the subject is a human subject. In various embodiments, the cancer is selected from but not limited to pancreatic cancer, gastric cancer, ovarian cancer, colorectal cancer, melanoma, leukemia, myelodysplastic syndrome, lung cancer, prostate cancer, brain cancer, bladder cancer, head-neck cancer, or rhabdomyosarcoma.

In another aspect, the present disclosure provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, and stem cell transplantation. In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, PD-L1, OX-40, CD137, TIGIT, GITR, LAGS, TIM-3, CD47, SIRPα, ICOS, and VISTA; treatment using bispecific T cell engaging antibodies (BiTE®) such as blinatumomab: treatment involving administration of biological response modifiers such as TNF family, IL-1, IL-4, IL-7, IL-12, IL-15, IL-17, IL-21, IL-22, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR: TLR7, TLR8, and TLR 9) agonists CpG and imiquimod; wherein the combination therapy provides increased effector cell killing of tumor cells, i.e., a synergy exists between the IL-2 variants and the immunotherapy when co-administered.

In another aspect, the disclosure provides uses of the IL-2 variants for the preparation of a medicament for the treatment of cancer.

In another aspect, the present disclosure provides isolated nucleic acid molecules comprising a polynucleotide encoding an IL-2 variant of the present disclosure. In another aspect, the present disclosure provides vectors comprising the nucleic acids described herein. In various embodiments, the vector is an expression vector. In another aspect, the present disclosure provides isolated cells comprising the nucleic acids of the disclosure. In various embodiments, the cell is a host cell comprising the expression vector of the disclosure. In another aspect, methods of making the IL-2 variants are provided by culturing the host cells under conditions promoting expression of the proteins or polypeptides.

### BRIEF DESCRIPTION OF THE FIGURES

FIG. 1 depicts the purity determined by SDS-PAGE (under non-reducing (lane 1) and reducing conditions (lane 2)) and monomer percentage assessed by SEC-HPLC of exemplary IL-2 variant Fc fusion proteins P-0635 (1A) and P-0704 (1B). P-0635 and P-0704 share the same amino acid substitution P65R in wild-type IL-2. P-0635 comprises a bivalent IL-2 variant fused to homodimer Fc, while P-0704 comprises a monovalent IL-2 variant fused to knob-into-hole heterodimeric Fc.

FIG. 2 depicts size exclusion chromatogram of exemplary IL-2 Fc fusion proteins P-0250 (2A), P-0318 (2B), P-0317 (2C), and P-0531 (2D) after protein A purification.

FIG. 3 depicts the impact of IL-2 valency on the binding strength of IL-2 Fc fusion proteins to IL-2Rα in ELISA. P-0531 and P-0689 share the same the developability-improving amino acid substitution S125I in wild-type IL-2. P-0531 comprises a bivalent IL-2 variant fused to homodimer Fc, while P-0689 comprises monovalent IL-2 fused to knob-into-hole heterodimeric Fc.

FIG. 4 depicts the impact of various mutations on the binding strength of IL-2 variant Fc fusions to IL-2Rα in ELISA. (4A) The IL-2 variant Fc fusions contain amino acid substitutions to T41; (4B) the IL-2 variant Fc fusions contain amino acid substitutions to Y107; (4C and 4D) the IL-2 variant Fc fusions contain amino acid substitutions to R38.

FIG. 5 depicts the impact of IL-2 E68 substitutions on the binding strength of IL-2 variant Fc fusions to IL-2Rα in ELISA.

FIG. 6 depicts the impact of IL-2 E62 substitutions on the binding strength of IL-2 variant Fc fusions to IL-2Rα in ELISA.

FIG. 7 depicts the impact of various IL-2 P65 substitutions on the binding strength of IL-2 variant Fc fusions to IL-2Rα in ELISA. (7A-7B) IL-2 P65 substitutions resulted in enhanced binding to IL-2Rα; (7C) IL-2 P65 substitutions resulted in reduced binding to IL-2Rα; (7D) IL-2 P65 substitutions resulted in complete loss of binding to IL-2Rα.

FIG. 8 depicts the effect of IL-2 amino acid substitution combinations on the binding strength to IL-2Rα in ELISA. (8A) The impact of IL-2 F42A substitution on the binding strength to IL-2Rα; (8B) Combination of F42A and CD25-disrupting substitution E62F resulted in complete loss of binding to IL-2Rα; (8C) Combination of F42A and CD25-disrupting substitution P65H resulted in complete loss of binding to IL-2Rα.

FIG. 9 depicts differential effects of IL-2 variants Fc fusion proteins on dose-dependent induction of STAT5 phosphorylation in CD4+ Treg cells in comparison with the wild-type fusion protein (P-0531) and a benchmark protein (P-0551) in human PBMC assay. The panel of IL-2 variants contain CD25-interfering mutations that resulted in enhanced, reduced, or abolished binding to IL-2Rα.

FIG. 10 depicts the full preservation of binding of a panel of IL-2 variant Fc fusion proteins to IL-2Rβγ in ELISA in comparison to the wild-type IL-2 fusion protein P-0531, and a benchmark protein P-0551. The panel of IL-2 variants contain CD25-interfering mutations that resulted in enhanced, reduced, or abolished binding to IL-2Rα.

FIG. 11 depicts that a panel of IL-2 variant Fc fusion proteins exhibited comparable activity in inducing Ki67 expression on CD8+ T cells (11A) and NK cells (11B) in human PBMC. The panel of IL-2 variants contain CD25-interfering mutations that resulted in enhanced, reduced, or abolished binding to IL-2Rα. Wild-type IL-2 fusion protein P-0531 and a benchmark protein P-0511 are included for comparison.

FIG. 12 depicts the impact of IL-2 valency on the activity in inducing Ki67 expression on CD8+ T cells in human PBMC. P-0531 and P-0689 are the bivalent and monovalent counterparts of wild-type IL-2 Fc fusion proteins. P-0635 and P-0704 are the bivalent and monovalent equivalents of IL-2 P65R Fc fusions.

FIG. 13 depicts the impact of various IL-2Rβ/γc-modulating amino acid substitutions or N-terminal deletions on the activity of inducing pSTAT5 expression on CD4+ T cells in comparison to their wild-type counterpart. (13A) IL-2 mutants with amino acid substitutions at position D20; (13B-13C) IL-2 mutants with amino acid substitutions at position L19; (13D) IL-2 Q126E mutation; and (13E) IL-2 mutants with N-terminal acid deletions.

FIG. 14 depicts the impact of IL-2Rβ- or γc-disrupting amino acid substitutions on binding strength to IL-2Rβγ in ELISA (14A) and on the activity in inducing Ki67 expression on CD8+ T cells in human PBMC (14B). P-0689 is a monovalent wild-type IL-2 Fc fusion protein and P-0704 is a monovalent IL-2 P65R Fc fusion that can no longer bind to IL-2Rα but retains full affinity and functional activity for the dimeric IL-2Rβγ receptor.

FIG. 15 depicts the impact of various IL-2Rβ-disrupting amino acid changes on the activity of IL-2 variant Fc fusions in inducing Ki67 expression on CD8+ T cells (15A), NK cells (15B), and CD4+ T cells (15C) in human PBMC. P-0704 and benchmark molecule (the monomeric version of P-0551) were included for comparison.

FIG. 16 depicts time-dependent effects of P-0704 on the expansion of Treg (16A), CD8+ T (16B), and NK cells (16C) in peripheral blood following a single injection in Balb/C mice. P-0704 is a monovalent IL-2 P65R Fc fusion; P-0689, a monovalent wild-type IL-2 Fc fusion protein was included for comparison. Blood was collected on days 3 and 5 for lymphocyte phenotyping by FACS analysis.

FIG. 17 depicts the impact of fusion format on dose-dependent induction of STAT5 phosphorylation on CD4+ Treg (17A), CD8+ T (17B), and NK cells (17C) in human PBMC assay. P-0704 is a monovalent IL-2 P65R Fc fusion, and P-0803 is an antibody fusion harboring the same IL-2 moiety.

FIG. 18 depicts differential effects of IL-2 variant antibody fusion proteins on dose-dependent induction of STAT5 phosphorylation on CD4+ Treg (18A), CD8+ T (18B), and NK cells (18C) in comparison with the wild type fusion protein (P-0837) in human PBMC assay. P-0838 harbors IL-2 P65Q mutation that significantly reduced binding ability to IL-2Rα, and P-0782 has an IL-2 P65R moiety with abolished binding to IL-2Rα.

FIG. 19 depicts impact of IL-2Rβ-modulating amino acid changes on the activity of IL-2 variant antibody fusions in stimulating STAT5 phosphorylation on CD8+ T (19A) and NK (19B), and in inducing Ki67 expression on CD8+ T (19C) and NK (19D) cells in human PBMC. All three compounds comprise P65R mutation in IL-2 moiety, and P-0786 and P-0783 contain additional IL-2Rβ-disrupting mutations L19Q and L19H, respectively.

FIG. 20 depicts impact of IL-2Rβ-modulating amino acid changes on the activity of IL-2 variant antibody fusions in stimulating STAT5 phosphorylation on CD4+ Treg (20A), CD8+ T (20B), and NK cells (20C), and in inducing Ki67 expression on CD8+ T (20D) and NK cells (20E) in human PBMC assay. All three compounds, P-0838, P-0790, and P-0787 comprise P65Q mutation in IL-2 moiety, and P-0790 and P-0787 contain additional IL-2Rβ-modulating mutations L19Q and L19H, respectively. P-0837 is the wild-type IL-2 fusion counterpart.

FIG. 21 depicts impact of IL-2Rβ-modulating amino acid changes on the activity of IL-2 variant antibody fusions in proliferating CTLL-2 cells. P-0782, P-0783, and P-0786 all comprise P65R mutation in IL-2 moiety; P-0786 and P-0783 contain additional IL-2Rβ-modulating mutations L19Q and L19H, respectively. P-0837 is the wild-type IL-2 fusion counterpart.

FIG. 22 depicts the minimal impact of fusion of IL-2 variants on direct binding (22A) and ligand competitive inhibition (22B) to the antibody arm in ELISA, and similarly, IL-2 variant human PD-1 antibody IL-2 showed similar binding as the parent antibody to PD1 expressed on cell surface analyzed by FACS analysis (FIG. 22C). P-0795 is a human PD-1 antagonist antibody, P-0803, P-0880, and P-0885 have monomeric IL-2 P65R variant covalently linked to the C-terminus of P-0795's heavy chain. P-0803 and P-0885 share the same IL-2 P65R/S125I substitutions but with different linkers ((G₃S)₂ and (G₄S)₃, respectively). P-0885 contains one additional L19Q mutation that P-0880. P-0704 and P-0859 are the Fc fusion counterparts of P-0880 and P-0885, respectively.

FIG. 23 depicts differential effects of IL-2 variant antibody fusion proteins on dose-dependent induction of STAT5 phosphorylation on CD4+ Treg (23A & 23B),) CD8+ T (23C & 23D), and NK cells (23E & 23F) in human PBMC. P-0803 and P-0804 are IL-2 variant human PD-1 antibody fusion proteins harboring P65R and L19H/P65R mutations, respectively. P-0782 is IL-2 P65R surrogate mouse PD-1 antibody fusion, and P-0783 contains an additional L19H mutation compared to P-0782.

FIG. 24 depicts the size exclusion chromatograms of IL-2 variant human PD-1 antibody fusion proteins, P-0840 (24A), P-0841 (24B), P-0803 (24C), and P-0880 (24D), after protein A purification.

FIG. 25 depicts the impact of linker length of IL-2 variants antibody fusion proteins on dose-dependent induction of STAT5 phosphorylation on CD8+ T (25A & 25B), and NK cells (25C & 25D) in human PBMC assay. P-0840 and P-0841 are both IL-2 L19Q/P650 variant hu man PD-1 antibody fusion proteins; P-0840 comprises a (G₃S)₂ linker while P-0841 has a (G₄S)₃ linker. Likewise, P-0803 and P-0880 are IL-2 P65R variant human PD-1 antibody fusion proteins; P-0803 comprises a (G₃S)₂ linker while P-0880 has a (G₄S)₃ linker.

FIG. 26 depicts the impact of IL-2Rβ-modulating amino acid changes on the activity of IL-2 variant human PD-1 antibody fusions in stimulating STAT5 phosphorylation on CD8+ T (26A) and NK cells (26B), and in inducing Ki67 expression on CD8+ T (26C) and NK cells (26D) in human PBMC. All three compounds, P-0880, P-0885, and P-0882 comprise P65R mutation in IL-2 moiety, while P-0885 and P-0882 contain additional IL-2Rβ-modulating mutations L19Q and L19H, respectively. P-0849 is the wild-type IL-2 fusion counterpart. All compounds have (G₄S)₃ linker connecting PD-1 antibody heavy chain and IL-2.

FIG. 27 depicts time-dependent effects of IL-2 variant surrogate mouse PD-1 antibody fusion proteins P-0782, P-0838, P-0781 (Benchmark), and P-0837 on Ki67 expression on CD8+ T (27A), and NK cells (27B), and effects on cell expansion of CD8 (27C) and NK cells (27D) following a single injection in C57BL6 mice. Cell expansion was expressed in cell number fold changes over the baseline. P-0782 comprises P65R mutation in IL-2 moiety, P-0838 contains P65Q mutation, P-0781 harbors a benchmark IL-2 variant that abolished binding to IL-2Rα, and P-0837 is the wild-type IL-2 fusion counterpart.

FIG. 28 depicts time- and does-dependent effects of IL-2 variant surrogate mouse PD-1 antibody fusion protein P-0786 on Ki67 expression on CD8+ T (28A), and NK cells (28B), and effects on cell expansion of CD8+ T (28C) and NK cells (28D) following a single injection in C57BL6 mice. Cell expansion was expressed in fold change in cell numbers over the baseline. P-0786 comprises L19Q/P65R mutation that renders abolished binding to IL-2Rα and reduced overall potency. P-0837, the wild-type IL-2 fusion counterpart, was included for comparison.

FIG. 29 depicts time- and does-dependent effects of IL-2 variant surrogate mouse PD-1 antibody fusion protein P-0783 on Ki67 expression on CD8+ T (29A), and NK cells (29B), and effects on cell expansion of CD8 (29C) and NK cells (29D) following a single injection in C57BL6 mice. Cell expansion was expressed in fold change in cell numbers over the baseline. P-0783 comprises L19H/P65R mutation that renders abolished binding to IL-2Rα and reduced overall potency. P-0837, the wild-type IL-2 fusion counterpart, was included for comparison.

FIG. 30 depicts body weight change in C57BL/6 mice treated with IL-2 variant surrogate mouse PD-1 antibody fusion proteins, P-0782, P-0786, and P-0783. All compounds comprise P65R mutation in the IL-2 moiety, P-0781 harbors a benchmark IL-2 variant that abolished binding to IL-2Rα, and P-0786 and P-0783 contain additional IL-2Rβ-disrupting mutations L19Q and L19H, respectively. Data are expressed as mean±SEM.

FIG. 31 depicts the antitumor efficacy (31A) and body weigh change (31B) of IL-2 variant surrogate mouse PD-1 antibody fusion proteins in subcutaneous B16F10 murine melanoma tumor model following a Q7D repeated dosing schedule. All three antibody fusion proteins contain IL-2 L65Q mutation to impair binding to IL-2Rα; P-0790 and P-0787 comprise additional L19Q and L19H mutations, respectively, to further modulate overall potency. Data are expressed as mean±SEM.

FIG. 32 depicts the antitumor efficacy (32A) and body weigh change (32B) of P-0787 at two different doses in subcutaneous B16F10 murine melanoma tumor model following a Q7D repeated dosing schedule. P-0787 is an IL-2 variant surrogate mouse PD-1 antibody fusion protein comprise L19H/P65Q mutations. Data are expressed as mean±SEM.

FIG. 33 depicts the antitumor efficacy of IL-2 variant surrogate mouse PD-1 antibody fusion proteins, P-0782 and P-0786, in subcutaneous B16F10 murine melanoma tumor model following a Q7D repeated dosing schedule. P-0722, the surrogate mouse PD-1 antibody, was included for comparison. Both P-0782 and P-0786 comprise IL-2Rα binding-abrogated mutation P65R, while P-0786 contains additional L19Q mutation to modulate overall potency. Data are expressed as mean±SEM.

FIG. 34 depicts dose-dependent inhibition of lung metastatic nodules by P-0790 in mouse B16F10 pulmonary metastasis model. (34A) Average lung nodule counts; (34B) Lung picture of a representative animal from each group. P-0790 is an IL-2 L19Q/P65Q surrogate mouse PD-1 antibody fusion protein with significantly impaired binding to IL-2Rα and modulated overall potency. Data are expressed as mean±SEM. Statistical analysis was performed by one-way anova followed by Tukey post hoc test. *p<0.05.

### MODE(S) FOR CARRYING OUT THE DISCLOSURE

The present invention relates to polypeptides which share primary sequence with human IL-2, except for one to several amino acids that have been mutated. IL-2 variants comprise mutations substantially reduce the ability of these polypeptides to stimulate Treg cells and make them more effective in the therapy of tumors. Also includes therapeutic uses of these mutated variants, used alone or in combination with vaccines, or TAA-targeting biologics, or immune checkpoint blocker, or as the building block in bifunctional molecule construct, for the therapy of diseases such as cancer or infections where the activity of regulatory T cells (Tregs) is undesirable. In another aspect the present invention relates to pharmaceutical compositions comprising the polypeptides disclosed. Finally, the present invention relates to the therapeutic use of the polypeptides and pharmaceutical compositions disclosed due to their selective modulating effect of the immune system on cancer and various infectious diseases.

### Definitions

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (amino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine (S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within +2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included.

Exemplary amino acid substitutions are set forth in Table 1.

TABLE 1

| Original Residues | Exemplary Substitutions | Preferred Substitutions |
|---|---|---|
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g. amino acid sequence) or the post-translational modifications (e.g. glycosylation) of a polypeptide.

The term “knob-into-hole modification” as used herein refers to a modification within the interface between two immunoglobulin heavy chains in the CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001).

The term “fusion protein” as used herein refers to a fusion polypeptide molecule comprising two or more genes that originally coded for separate proteins, wherein the components of the fusion protein are linked to each other by peptide-bonds, either directly or through peptide linkers. The term “fused” as used herein refers to components that are linked by peptide bonds, either directly or via one or more peptide linkers.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 2-20 amino acids. Peptide linkers are known in the art or are described herein. Suitable, non-immunogenic linker peptides include, for example, (G₄S)ₙ, (SG₄)ₙ or G₄(SG₄)ₙ peptide linkers. “n” is generally a number between 1 and 10, typically between 2 and 4.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, SIRP, CD47 and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

The term “Fc domain” or “Fc region” as used herein is used to define a C-terminal region of an immunoglobulin heavy chain that contains at least a portion of the constant region. The term includes native sequence Fc regions and variant Fc regions. An IgG Fc region comprises an IgG CH2 and an IgG CH3 domain. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical immunoglobulin heavy chains as herein described. Unless otherwise specified herein, numbering of amino acid residues in the Fc region or constant region is according to the EU numbering system.

The term “effector functions” as used herein refers to those biological activities attributable to the Fc region of an immunoglobulin, which vary with the immunoglobulin isotype. Examples of immunoglobulin effector functions include: CIq binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. Effector functions may also refer to similar immune responses elicited by effector immune cells such as CD8 and NK cell.

The term “regulatory T cell” or “Treg cell” as used herein is meant a specialized type of CD4+ T cell that can suppress the responses of other T cells (effector T cells). Treg cells are characterized by expression of CD4, the α-subunit of the IL-2 receptor (CD25), and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors.

The term “conventional CD4+ T cells” as used herein is meant CD4+ T cells other than regulatory T cells. The conventional CD4+ T cells expression of CD3 and CD4. At naïve and unstimulated condition, they do not express the α-subunit of the IL-2 receptor (CD25) but express the βγ-subunit of the IL-2 receptor.

The term “CD8 T cells” are a type of cytotoxic T lymphocytes characterized by expression of CD3 and CD8. CD8 T cells mainly express the βγ-subunit of the IL-2 receptor and play a critical role in killing cancer cells, cells that are infected with viruses, or cells that are damaged in other ways

The term “NK cells” are a type of cytotoxic lymphocyte critical to the innate immune system. NK cells mainly express the βγ-subunit of the IL-2 receptor and provide rapid responses to virus-infected cells and tumor formation.

As used herein, “specific binding” is meant that the binding is selective for the antigen and can be discriminated from unwanted or non-specific interactions. The ability of an immunoglobulin to bind to a specific antigen can be measured either through an enzyme-linked immunosorbent assay (ELISA) or other techniques familiar to one of skill in the art, e.g. Surface Plasmon Resonance (SPR) technique.

The terms “affinity” or “binding affinity” as used herein refers to the strength of the sum total of non-covalent interactions between a single binding site of a molecule (e.g. an antibody) and its binding partner (e.g. an antigen). The affinity of a molecule X for its partner Y can generally be represented by the dissociation constant (KD), which is the ratio of dissociation and association rate constants (koff and kon, respectively). A particular method for measuring affinity is Surface Plasmon Resonance (SPR).

The term “reduced binding”, as used herein refers to a decrease in affinity for the respective interaction, as measured for example by SPR. Conversely, “increased binding” refers to an increase in binding affinity for the respective interaction.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

By “polyethylene glycol” or “PEG” is meant a polyalkylene glycol compound or a derivative thereof, with or without coupling agents or derivatization with coupling or activating moieties (e.g., with aldehyde, hydroxysuccinimidyl, hydrazide, thiol, triflate, tresylate, azirdine, oxirane, orthopyridyl disulphide, vinylsulfone, iodoacetamide or a maleimide moiety). In various embodiments, PEG includes substantially linear, straight chain PEG, branched PEG, or dendritic PEG. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, organophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA. The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence-dependent and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3.sup.rd ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses, and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers, and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The terms “label” or “labeled” as used herein refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In various embodiments, labels are attached by spacer arms of various lengths to reduce potential steric hindrance.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variations.

### IL-2

Interleukin-2 (IL-2), a classic Th1 cytokine, is produced by T cells after activation through the T-cell antigen receptor and the co-stimulatory molecule CD28. The regulation of IL-2 occurs through activation of signaling pathways and transcription factors that act on the IL-2 promoter to generate new gene transcription, but also involves modulation of the stability of IL-2 mRNA. IL-2 binds to a multichain receptor, including a highly regulated α chain and β and γ chains that mediate signaling through the Jak-STAT pathway. IL-2 delivers activation, growth, and differentiation signals to T cells, B cells, and NK cells. IL-2 is also important in mediating activation-induced cell death of T cells, a function that provides an essential mechanism for terminating immune responses. A commercially available unglycosylated human recombinant IL-2 product, aldesleukin (available as the PROLEUKIN® brand of des-alanyl-1, serine-125 human interleukin-2 from Prometheus Laboratories Inc., San Diego Calif.), has been approved for administration to patients suffering from metastatic renal cell carcinoma and metastatic melanoma. IL-2 has also been suggested for administration in patients suffering from or infected with hepatitis C virus (HCV), human immunodeficiency virus (HIV), acute myeloid leukemia, non-Hodgkin's lymphoma, cutaneous T-cell lymphoma, juvenile rheumatoid arthritis, atopic dermatitis, breast cancer and bladder cancer. Unfortunately, short half-life and severe toxicity limits the optimal dosing of IL-2.

As used herein, the terms “native IL-2” and “native interleukin-2” in the context of proteins or polypeptides refer to any naturally occurring mammalian interleukin-2 amino acid sequences, including immature or precursor and mature forms. Non-limiting examples of GenBank Accession Nos. for the amino acid sequence of various species of native mammalian interleukin-2 include NP 032392.1 (Mus musculus, immature form), NP_001040595.1 (Macaca mulatta, immature form), NP_000577.2 (human, precursor form), CAA01199,1 (human, immature form), AAD48509.1 (human, immature form), and AAB20900.1 (human). In various embodiments of the present invention, native IL-2 is the immature or precursor form of a naturally occurring mammalian IL-2. In other embodiments, native IL-2 is the mature form of a naturally occurring mammalian IL-2. In various embodiments, native IL-2 is the precursor form of naturally occurring human IL-2. In various embodiments, native IL-2 is the mature form of naturally occurring human IL-2. In various embodiments, the IL-2-based domain D2 is derived from the amino acid sequence of the human IL-2 precursor sequence set forth in SEQ ID NO: 1:

(SEQ ID NO: 1)
MYRMQLLSCIALSLALVTNSAPTSSSTKKTQLQLE
HLLLDLQMILNGINNYKNPKLTRMLTFKFYMPKKA
TELKHLQCLEEELKPLEEVLNLAQSKNFHLRPRDL
ISNINVIVLELKGSETTFMCEYADETATIVEFLNR
WITFCQSIISTLT

In various embodiments, the IL-2-based domain D2 comprises the amino acid sequence of the human IL-2 mature form wild-type sequence set forth in SEQ ID NO: 3, which contains substitution of cysteine at position 125 to serine, but does not alter IL-2 receptor binding compared to the naturally occurring IL-2:

(SEQ ID NO: 3)
APTSSSTKKTQLQLEHLLLDLQMILNGINNYKNPK
LTRMLTFKFYMPKKATELKHLQCLEEELKPLEEVL
NLAQSKNFHLRPRDLISNINVIVLELKGSETTFMC
EYADETATIVEFLNRWITFSQSIISTLT

### IL-2 Variants

The present invention relates to polypeptides which share primary sequence with human IL-2, except for one to several amino acids that have been mutated. One panel of IL-2 variants comprise mutations substantially reduce the ability of these polypeptides to stimulate Treg cells and make them more effective in the therapy of tumors. Also includes therapeutic uses of these mutated variants, used alone or in combination with vaccines, or TAA-targeting biologics, or immune checkpoint blocker, or as the building block in bifunctional molecule construct, for the therapy of diseases such as cancer or infections where the activity of regulatory T cells (Tregs)—is undesirable. In another aspect the present invention relates to pharmaceutical compositions comprising the polypeptides disclosed. Finally, the present invention relates to the therapeutic use of the polypeptides and pharmaceutical compositions disclosed due to their selective modulating effect of the immune system on diseases like autoimmune and inflammatory disorders or cancer and various infectious diseases.

The present invention relates to polypeptides of 100 to 500 amino acids in length, preferably of 140 residues size whose apparent molecular weight is at least 15 kD. These polypeptides maintain high sequence identity, more than 90%, with native IL-2. In a region of their sequence, these polypeptides are mutated introducing amino acid residues different from those in the same position in the native IL-2.

The polypeptides of the present invention may be referred to as immunomodulatory polypeptides, IL-2 analogs or IL-2 variants, among other names. These polypeptides are designed based on the 3D structure of the IL-2 receptor complex (available in PDB public database), introducing mutations mainly in the positions of the IL-2 corresponding to amino acids interacting with IL-2 receptor subunit α.

In various embodiments, the IL-2 variant (or mutant) comprises a sequence derived from the sequence of the mature human IL-2 polypeptide as set forth in SEQ ID NO: 3. In various embodiments, the IL-2 variant comprises a different amino acid sequence than the native (or wild type) IL-2 protein. In various embodiments, the IL-2 variant interacts with the IL-2 receptor polypeptide and functions as an IL-2 agonist or antagonist. In various embodiments, the IL-2 variants with agonist activity have super agonist activity. In various embodiments, the IL-2 variant can function as an IL-2 agonist or antagonist independent of its association with IL-2Rα. IL-2 agonists are exemplified by comparable or increased biological activity compared to wild type IL-2. IL-2 antagonists are exemplified by decreased biological activity compared to wild type IL-2 or by the ability to inhibit IL-2-mediated responses. In various embodiments, the sequence of the IL-2 variant has at least one amino acid change, e.g. substitution or deletion, compared to the native IL-2 sequence, such changes resulting in IL-2 agonist or antagonist activity. In various embodiments, the IL-2 variants have the amino acid sequences set forth in SEQ ID NOS: 31-66 with reduced/abolished binding to IL-2Rα to selectively activate and proliferate effector T cells (Teff). In various embodiments, the IL-2 variants have the amino acid sequences set forth in SEQ ID NOS: 111-120 comprising IL-2β or γc-modulating mutations in addition to mutations that cause reduced/abolished binding to IL-2Rα to selectively activate and proliferate effector T cells with attenuated potency in order to reduce IL-2β or γc associated toxicity, attenuate cell exhaustion and improved durable pharmacodynamics. In various embodiments, the IL-2 variants have the amino acid sequences in SEQ ID NOS: 189 (amino acids 462-586), 190 (amino acids 462-585), and 191 (amino acids 462-584) comprising N-terminal deletions in addition to mutations that cause reduced/abolished binding to IL-2Rα to selectively activate and proliferate effector T cells with attenuated potency. In various embodiments, the IL-2 variants with the amino acid sequences set forth in SEQ ID NOS: 31-66, 111-120, and amino acids 9-133, 10-133, and 11-133 of SEQ ID NOS: 47 also comprise S125I amino acid substitution to improve the developability profiles of IL-2 and the corresponding fusion proteins.

Exemplary IL-2 variants with amino acid substitutions introduced at the interface with the IL-2Rα are provided in Table 2:

TABLE 2
IL-2 variants or fusion constructs comprising mutation(s) to
amino acids interacting with receptor subunit α. All variants
comprise the developability-improving substitution (S125I).

| | | Bivalent IL-2 | | Monovalent IL-2 | |
|---|---|---|---|---|---|
| | | Fc fusion | | Fc fusion | |
| Amino acid | SEQ ID: | Protein | SEQ | Protein | SEQ |
| substitutions | NO | ID | ID NO: | ID | ID NO: |
| F42A | 31 | P-0613 | 69 | X | X |
| R38F | 32 | P-0614 | 70 | X | X |
| R38G | 33 | P-0615 | 71 | X | X |
| R38A | 34 | P-0602 | 72 | X | X |
| T41A | 35 | P-0603 | 73 | X | X |
| T41G | 36 | P-0604 | 74 | X | X |
| T41V | 37 | P-0605 | 75 | X | X |
| F44G | 38 | P-0606 | 76 | X | X |
| F44V | 39 | P-0607 | 77 | X | X |
| E62A | 40 | P-0624 | 78 | X | X |
| E62F | 41 | P-0625 | 79 | X | X |
| E62H | 42 | P-0626 | 80 | X | X |
| E62L | 43 | P-0627 | 81 | X | X |
| P65G | 44 | P-0608 | 82 | X | X |
| P65E | 45 | P-0633 | 83 | X | X |
| P65H | 46 | P-0634 | 84 | X | X |
| P65R | 47 | P-0635 | 85 | P-0704 | 96 + 10 |
| P65A | 48 | X | X | P-0706 | 97 + 10 |
| P65K | 49 | X | X | P-0707 | 98 + 10 |
| P65N | 50 | X | X | P-0708 | 99 + 10 |
| P65Q | 51 | X | X | P-0709 | 100 + 10 |
| E68A | 52 | P-0628 | 86 | X | X |
| E68F | 53 | P-0629 | 87 | X | X |
| E68H | 54 | P-0630 | 88 | X | X |
| E68L | 55 | P-0631 | 89 | X | X |
| E68P | 56 | P-0632 | 90 | X | X |
| Y107G | 57 | P-0609 | 91 | X | X |
| Y107H | 58 | P-0610 | 92 | X | X |
| Y107L | 59 | P-0611 | 93 | X | X |
| Y107V | 60 | P-0612 | 94 | X | X |
| IL-2 Variant | X | X | 95 | X | X |
| Benchmark | | | | | |
| F42A/E62F | 61 | X | X | P-0702 | 101 + 10 |
| F42A/E62A | 62 | X | X | P-0766 | 102 + 10 |
| F42A/E62H | 63 | X | X | P-0767 | 103 + 10 |
| F42A/P65H | 64 | X | X | P-0703 | 104 + 10 |
| F42A/P65R | 65 | X | X | P-0705 | 105 + 10 |
| F42A/P65A | 66 | X | X | P-0765 | 106 + 10 |

The main aspect of the present invention is to improve IL-2 selectivity relative to wild-type IL-2 for cells expressing IL-2Rβγ (but not IL-2Rα) over cells expressing IL-2Rαβγ for cancer therapy. One approach used by the present inventors is to generate highly selective IL-2-Fc-fusion proteins through introduction of CD25-disrupting mutations into the cytokine component. Selection of CD25-disrupting mutations was based on inspection of the IL-2/IL-2R co-crystal structure (PDB code 2651). Multiple amino acid substitutions to one or two relevant residues at the interface with the IL-2 receptor a subunit, including R38, T41, F42, F44, E62, P65, E68, and Y107, were introduced aiming to reduce or abolish binding to IL-2Rα. These constructs also contained S125I mutation for significantly improved developability. Additionally, impairment of IL-2 variants in binding to IL-2Rα+ pulmonary endothelial cells is expected to prevent endothelial cell damage and significantly reduce VLS. Furthermore, impairment of CD25 binding is also expected to reduce CD25 antigen sink and enrich the cytokine occupancy to IL-2Rβγ-expressing cells and consequently enhanced in vivo response and tumor killing efficacy.

As all the targeted IL-2 residues, R38, T41, F42, F44, E62, P65, E68, and Y107, are at the interface with IL-2Rα and form either hydrogen bond/salt bridge or hydrophobic interactions with multiple IL-2Rα residues (Mathias Rickert, et al. (2005) Science 308, 1477-80), it was reasoned that the IL-2 variants listed in Table 2 and similar are expected to disrupt interaction with IL-2Rα and resulted in IL-2 variants with reduced or abolished binding to IL-2Rα. However, it was discovered that mutations at different sites and different substitutions at the same site could result in drastic differences in affecting IL-2Rα binding, which could not be predicted based on the structure-based mutagenesis approach, and some are particularly unexpected (refer to examples 4 and 5).

Further, it was reasoned that IL-2Rβγ-modulating substitutions can be further incorporated to attenuate overall potency for optimal activity. Agonists of IL-2Rβγ modulated potency may prevent over-activation of the cytotoxic lymphocytes and minimize “on-target” and “off tissue” toxicity. In addition, overstimulation induced cell exhaustion and apoptosis can be minimized. Further, attenuation of binding affinity of cytokine signaling molecule can reduce receptor mediated internalization, decrease unwanted target sink and lead to persistent receptor activation and durable pharmacodynamics and pharmacokinetics; Consequently, IL-2Rβγ-modulating substitutions can potentially reduce toxicity and improve pharmacokinetics and pharmacodynamics as well as therapeutic index.

Exemplary IL-2 variants with amino acid substitutions comprising IL-2β or γc-disrupting mutations to IL-2 variants with reduced/abolished binding to IL-2Rα are provided in Table 3:

TABLE 3
Introduction of IL-2Rβ or γc-disrupting substitutions
to IL-2 variants with reduced/abolished binding to IL-2Rα. All
variants comprise the developability-improving substitution (S125I).

| | | Monovalent IL-2 | | Bivalent IL-2 | |
|---|---|---|---|---|---|
| | | Fc fusion | | Fc fusion | |
| Amino acid | SEQ ID: | Protein | SEQ | Protein | SEQ |
| substitutions | NO | ID | ID NO: | ID | ID NO: |
| L19H/P65R | 111 | P-0731 | 121 + 10 | P-0758 | 131 |
| L19Q/P65R | 112 | P-0759 | 122 + 10 | P-0760 | 132 |
| L19Y/P65R | 113 | P-0761 | 123 + 10 | P-0762 | 133 |
| L19H/P65Q | 114 | P-0811 | 124 + 10 | X | X |
| L19H/P65H | 115 | P-0812 | 125 + 10 | X | X |
| L19H/P65N | 116 | P-0813 | 126 + 10 | X | X |
| L19Q/P65Q | 117 | P-0814 | 127 + 10 | X | X |
| L19Q/P65H | 118 | P-0815 | 128 + 10 | X | X |
| L19Q/P65N | 119 | P-0816 | 129 + 10 | X | X |
| P65R/Q126E | 120 | P-0732 | 130 | X | X |

The present invention also includes additional modifications to the class of IL-2 variants mentioned above and especially to those described in Table 2 and Table 3, including deletions of 8, or 9, or 10 N-terminal residues to the IL-2 variants mentioned above to selectively activate and proliferate effector T cells with various level of attenuated potency. Any further combination mutants come with the spirit and scope of the present invention whether it is to alter their affinity to specific components of the IL-2 receptor, or to improve their in vivo pharmacodynamics: increase half-life or reduce their internalization by T cells. These additional mutations may be obtained by rational design with bioinformatics tools, or by using combinatorial molecular libraries of different nature (phage libraries, libraries of gene expression in yeast or bacteria). In another aspect the present invention relates to a fusion protein comprising any of the immunomodulatory polypeptides described above, coupled to a carrier protein. The carrier protein can be Albumin or the Fc region of human immunoglobulins.

In various embodiments, IL-2RαSushi having the amino acid sequence set forth in SEQ ID NO: 170, was linked between IL-2 and Fc domains using linkers of various lengths and compositions. Fc domain can be in the N-terminus or C-terminus. IL-2-IL-2RαSushi-Fc fusion protein have the amino acid sequence set forth in SEQ ID NOS: 171-172 is expected to have reduced binding to IL-2Rα to selectively activate and proliferate effector T cells.

In various embodiments, IL-2 and IL-2RαSushi form non-covalent complexation. IL-2 was fused to either N- or C-terminus of a Hole-Fc chain (SEQ ID NO: 10), and IL-2RαSushi was fused to either N- or C-terminus of a Knob-Fc chain (SEQ ID NO: 9). Non-covalent C-terminal IL-2-IL-2RαSushi-Fc fusion protein have the amino acid sequence set forth in SEQ ID NOS: 173-174.

TABLE 4
IL-2 and IL-2RαSushi covalently linked or
non-covalently complexed as Fc fusion proteins

| Construction design | Fusion protein ID | SEQ ID NO: |
|---|---|---|
| IL-2 linked to IL-2RαSushi at | P-0327 | 171 |
| C-terminal of Fc | | |
| IL-2 linked to IL-2RαSushi at | P-0422 | 172 |
| N-terminal of Fc | | |
| IL-2 and IL-2RαSushi non- | P-0482 | 173 + 174 |
| covalent complexed via | | |
| heterodimeric Fc | | |

### Fc Domains

Immunoglobulins of IgG class are among the most abundant proteins in human blood. Their circulation half-lives can reach as long as 21 days. Fusion proteins have been reported to combine the Fc regions of IgG with the domains of another protein, such as various cytokines and receptors (see, for example, Capon et al., Nature, 337:525-531, 1989; Chamow et al., Trends Biotechnol, 14:52-60, 1996); U.S. Pat. Nos. 5,116,964 and 5,541,087). The prototype fusion protein is a homodimeric protein linked through cysteine residues in the hinge region of IgG Fc, resulting in a molecule similar to an IgG molecule without the heavy chain variable and CH1 domains and light chains. The dimer nature of fusion proteins comprising the Fc domain may be advantageous in providing higher order interactions (i.e. bivalent or bispecific binding) with other molecules. Due to the structural homology, Fc fusion proteins exhibit in vivo pharmacokinetic profile comparable to that of human IgG with a similar isotype.

The term “Fc” refers to molecule or sequence comprising the sequence of a non-antigen-binding fragment of whole antibody, whether in monomeric or multimeric form. The original immunoglobulin source of the native Fc is preferably of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms. Fc domains containing binding sites for Protein A, Protein G, various Fc receptors and complement proteins.

In various embodiments, the term “Fc variant” refers to a molecule or sequence that is modified from a native Fc but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference. Furthermore, a native Fc comprises sites that may be removed because they provide structural features or biological activity that are not required for the fusion molecules of the present invention. Thus, in various embodiments, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by recombinant gene expression or by other means. In various embodiments, an “Fc domain” refers to a dimer of two Fc domain monomers (SEQ ID NO: 6) that generally includes full or part of the hinge region. In various embodiments, an Fc domain may be mutated to lack effector functions. In various embodiments, each of the Fc domain monomers in an Fc domain includes amino acid substitutions in the CH2 antibody constant domain to reduce the interaction or binding between the Fc domain and an Fcγ receptor. In various embodiments, each subunit of the Fc domain comprises three amino acid substitutions that reduce binding to an activating Fc receptor and/or effector function wherein said amino acid substitutions are L234A, L235A and G237A (SEQ ID NO: 7).

In various embodiments, each of the two Fc domain monomers in an Fc domain includes amino acid substitutions that promote the heterodimerization of the two monomers. In various other embodiments, heterodimerization of Fc domain monomers can be promoted by introducing different, but compatible, substitutions in the two Fc domain monomers, such as “knob-into-hole” residue pairs. The “knob-into-hole” technique is also disclosed in U.S. Pat. No. 8,216,805. In yet another embodiment, one Fc domain monomer includes the knob mutation T366W and the other Fc domain monomer includes hole mutations T366S, L358A, and Y407V. In various embodiments, two Cys residues were introduced (S354C on the “knob” and Y349C on the “hole” side) that form a stabilizing disulfide bridge (SEQ ID NOS: 9 and 10). The use of heterodimeric Fc may result in monovalent IL-2 variant.

In various embodiments, the Fc domain sequence used to make dimeric IL-2 variant Fc fusions is the human IgG1-Fc domain sequence set forth in SEQ ID NO: 7:

(SEQ ID NO: 7)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPSRDELTK
NQVSLTCLVKGFYPSDIAVEWESNGQPENNYKTTP
PVLDSDGSFFLYSKLTVDKSRWQQGNVFSCSVMHE
ALHNHYTQKSLSLSPG

wherein SEQ ID NO: 7 contains amino acid substitutions (underlined) that ablate FcγR and C1q binding.

In various embodiments, the Fc domain sequence used to make dimeric IL-2 Fc fusion proteins is the IgG1-Fc domain sequences set forth in SEQ ID NO: 8:

(SEQ ID NO: 106)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPSRDELTK
NQVSLTCLVKGFYPSDIAVEWESNGQPENNYKTTP
PVLDSDGSFFLYSKLTVDKSRWQQGNVFSCSVMHE
ALHAHYTQKSLSLSPG

wherein SEQ ID NO: 8 contains amino acid substitutions (underlined) that ablate FcγR and C1q binding and amino acid substitution (bold) to extend half-life.

In various embodiments, the heterodimeric Fc domain sequence used to make monomeric IL-2 variant fusions is the Knob-Fc domain sequence set forth in SEQ ID NO: 9:

(SEQ ID NO: 9)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVCTLPPSREEMTK
NQVSLWCLVKGFYPSDIAVEWESNGQPENNYKTTP
PVLDSDGSFFLYSKLTVDKSRWQQGNVFSCSVMHE
ALHNHYTQKSLSLSPG

wherein SEQ ID NO: 9 contains amino acid substitutions (underlined) that ablate FcγR and C1q binding.

In various embodiments, the heterodimeric Fc domain sequence used to make IL-2 variants is the Hole-Fc domain sequence set forth in SEQ ID NO: 10:

(SEQ ID NO: 10)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPCREEMTK
NQVSLSCAVKGFYPSDIAVEWESNGQPENNYKTTP
PVLDSDGSFFLVSKLTVDKSRWQQGNVFSCSVMHE
ALHNHYTQKSLSLSPG

wherein SEQ ID NO: 10 contains amino acid substitutions (underlined) that ablate FcγR and C1q binding.

In various embodiments, the heterodimeric Fc domain used to make monomeric IL-2 Fc fusion proteins is the Knob-Fc domain of reduced/abolished effector function and extended half-life with the amino acid sequence set forth in SEQ ID NO: 134:

(SEQ ID NO: 134)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVCTLPPSREEMTK
NQVSLWCLVKGFYPSDIAVEWESNGQPENNYKTTP
PVLDSDGSFFLYSKLTVDKSRWQQGNVFSCSVMHE
ALHAHYTQKSLSLSPG

wherein SEQ ID NO: 134 contains amino acid substitutions (underlined) that ablate FcγR and C1q binding and amino acid substitution (bold) to extend half-life.

In various embodiments, the heterodimeric Fc domain used to make monomeric IL-2 Fc fusion proteins is the Hole-Fc domain of reduced/abolished effector function and extended half-life with the amino acid sequence set forth in SEQ ID NO: 135:

(SEQ ID NO: 135)
DKTHTCPPCPAPEAAGAPSVFLFPPKPKDTLMISR
TPEVTCVVVDVSHEDPEVKFNWYVDGVEVHNAKTK
PREEQYNSTYRVVSVLTVLHQDWLNGKEYKCKVSN
KALPAPIEKTISKAKGQPREPQVYTLPPCREEMTK
NQVSLSCAVKGFYPSDIAVEWESNGQPENNYKTTP
PVLDSDGSFFLVSKLTVDKSRWQQGNVFSCSVMHE
ALHAHYTQKSLSLSPG

wherein SEQ ID NO: 135 contains amino acid substitutions (underlined) that ablate FcγR and C1q binding and amino acid substitution (bold) to extend half-life.

### Antibodies as Targeting Moieties

In various embodiments, the IL-2 variant constructs of the present invention comprise a targeting moiety in the form of an antibody, an antibody fragment, a protein or a peptide binding to a molecule enriched in the cancer tissue, such as a tumor associated antigen (TAA).

The TAA can be any molecule, macromolecule, combination of molecules, etc. against which an immune response is desired. The TAA can be a protein that comprises more than one polypeptide subunit. For example, the protein can be a dimer, trimer, or higher order multimer. In various embodiments, two or more subunits of the protein can be connected with a covalent bond, such as, for example, a disulfide bond. In various embodiments, the subunits of the protein can be held together with non-covalent interactions. Thus, the TAA can be any peptide, polypeptide, protein, nucleic acid, lipid, carbohydrate, or small organic molecule, or any combination thereof, against which the skilled artisan wishes to induce an immune response. In various embodiments, the TAA is a peptide that comprises about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 400, about 500, about 600, about 700, about 800, about 900 or about 1000 amino acids. In various embodiments, the peptide, polypeptide, or protein is a molecule that is commonly administered to subjects by injection. In various embodiments, after administration, the tumor-specific antibody or binding protein serves as a targeting moiety to guide the IL-2 variant to the diseased site, such as a cancer site, where the active domain can be released and interact with its cognate receptors on diseased cells.

Any of the foregoing markers can be used as TAAs targets for the IL-2 variants of this invention. In various embodiments, the one or more TAA, TAA variant, or TAA mutant contemplated for use in the IL-2 variant constructs and methods of the present disclosure is selected from, or derived from, the list provided in Table 5.

TABLE 5

| Tumor Associated Antigen | RefSeq (protein) |
|---|---|
| Her2/neu | NP_001005862 |
| Her3 | NP_001005915 |
| Her4 | NP_001036064 |
| EGF | NP_001171601 |
| EGFR | NP_005219 |
| CD2 | NP_001758 |
| CD3 | NM_000732 |
| CD5 | NP_055022 |
| CD7 | NP_006128 |
| CD13 | NP_001141 |
| CD19 | NP_001171569 |
| CD20 | NP_068769 |
| CD21 | NP_001006659 |
| CD22 | NP_001762 |
| CD23 | NP_001193948 |
| CD30 | NP_001234 |
| CD33 | NP_001234.3 |
| CD34 | NP_001020280 |
| CD38 | NP_001766 |
| CD40 | NP_001241 |
| CD46 | NP_002380 |
| CD55 | NP_000565 |
| CD59 | NP_000602 |
| CD69 | NP_001772 |
| CD70 | NM_001252 |
| CD71 | NP_001121620 |
| CD80 | NP_005182 |
| CD97 | NP_001020331 |
| CD117 | NP_000213 |
| CD127 | NP_002176 |
| CD134 | NP_003318 |
| CD137 | NP_001552 |
| CD138 | NP_001006947 |
| CD146 | NP_006491 |
| CD147 | NP_001719 |
| CD152 | NP_001032720 |
| CD154 | NP_000065 |
| CD195 | NP_000570 |
| CD200 | NP_001004196 |
| CD212 | NP_001276952 |
| CD223 | NP_002277 |
| CD253 | NP_001177871 |
| CD272 | NP_001078826 |
| CD276 | NP_001019907 |
| CD278 | NP_036224 |
| CD279 (PD-1) | NP_005009 |
| TIGIT | NP_776160 |
| CD309 (VEGFR2) | NP_002244 |
| DR6 | NP_055267 |
| CD274 (PD-L1) | NP_001254635 |
| Kv1.3 | NP_002223 |
| 5E10 | NP_006279 |
| MUC1 | NP_001018016 |
| uPA | NM_002658 |
| SLAMF7 (CD319) | NP_001269517 |
| MAGE 3 | NP_005353 |
| MUC 16 (CA-125) | NP_078966 |
| KLK3 | NP_001025218 |
| K-ras | NP_004976 |
| Mesothelin | NP_001170826 |
| p53 | NP_000537 |
| Survivin | NP_001012270 |
| G250 (Renal Cell Carcinoma Antigen) | GenBank CAB82444.1 |
| PSMA | NP_001014986 |
| HLA-DR | NP_001020330 |
| 1D10 | NP_114143 |
| Collagen Type I | NP_000079 |
| Collagen Type II | NP_000080 |
| Fibronectin | XP_005246463 |
| Tenascin | NP_002151 |
| Fibroblast Activation Protein (FAP) | NM_004460 |
| Matrix metalloproteinase-14 (MMP-14) | NP_004986 |
| Legumain | NP_001008530 |
| Matrix Metalloproteinase-2 (MMP-2) | NP_001121363 |
| Matrix Metalloproteinase-9 (MMP-9) | NP_004985 |
| Siglec-7 | NP_055200 |
| Siglec-9 | NP_001185487 |
| Siglec-15 | NP_998767 |

In various embodiments, the IL-2 variants of the present invention can be attached to targeting/dual functional moiety that is an antibody, an antibody fragment, a protein or a peptide targeting immune checkpoint modulators.

A number of immune-checkpoint protein antigens have been reported to be expressed on various immune cells, including, e.g., SIRP (expressed on macrophage, monocytes, dendritic cells), CD47 (highly expressed on tumor cells and other cell types), VISTA (expressed on monocytes, dendritic cells, B cells, T cells), CD152 (expressed by activated CD8+ T cells, CD4+ T cells and regulatory T cells), CD279 (expressed on tumor infiltrating lymphocytes, expressed by activated T cells (both CD4 and CD8), regulatory T cells, activated B cells, activated NK cells, anergic T cells, monocytes, dendritic cells), CD274 (expressed on T cells, B cells, dendritic cells, macrophages, vascular endothelial cells, pancreatic islet cells), and CD223 (expressed by activated T cells, regulatory T cells, anergic T cells, NK cells, NKT cells, and plasmacytoid dendritic cells)(see, e.g., Pardoll, D., Nature Reviews Cancer, 12:252-264, 2012). Antibodies that bind to an antigen which is determined to be an immune-checkpoint protein are known to those skilled in the art. For example, various anti-CD276 antibodies have been described in the art (see, e.g., U.S. Pat. Public. No. 20120294796 (Johnson et al) and references cited therein); various anti-CD272 antibodies have been described in the art (see, e.g., U.S. Pat. Public. No. 20140017255 (Mataraza et al) and references cited therein); various anti-CD152/CTLA-4 antibodies have been described in the art (see, e.g., U.S. Pat. Public. No. 20130136749 (Korman et al) and references cited therein); various anti-LAG-3/CD223 antibodies have been described in the art (see, e.g., U.S. Pat. Public. No. 20110150892 (Thudium et al) and references cited therein); various anti-CD279 (PD-1) antibodies have been described in the art (see, e.g., U.S. Pat. No. 7,488,802 (Collins et al) and references cited therein); various anti-CD274 (PD-L1) antibodies have been described in the art (see, e.g., U.S. Pat. Public. No. 20130122014 (Korman et al) and references cited therein); various anti-TIM-3 antibodies have been described in the art (see, e.g., U.S. Pat. Public. No. 20140044728 (Takayanagi et al) and references cited therein); and various anti-B7-H4 antibodies have been described in the art (see, e.g., U.S. Pat. Public. No. 20110085970 (Terrett et al) and references cited therein). Each of these references is hereby incorporated by reference in its entirety for the specific antibodies and sequences taught therein.

In various embodiments, IL-2 fusion partner can be an antibody, antibody fragment, or protein or peptide that exhibit binding to an immune-checkpoint protein antigen that is present on the surface of an immune cell. In various embodiments, the immune-checkpoint protein antigen is selected from the group consisting of, but not limited to, PD1 (CD279), PDL-1 (CD274), CD276, CD272, CD152 (CTLA-4), CD223, CD279, CD274, CD40, SIRPα, CD47, OX-40, GITR, ICOS, CD27, 4-1 BB, TIM-3, B7-H3, B7-H4, TIGIT and VISTA.

In various embodiments, the antibody is an antagonistic FAP antibody or antibody fragment. In various embodiments, the antibody is a humanized antagonistic FAP antibody comprising the variable domain sequences set forth in SEQ ID NOS: 136 and 137. In various embodiments, the heterologous protein is an antibody or an antibody fragment to an immune checkpoint modulator. In various embodiments, the antibody is an antagonistic human TIGIT antibody. In various embodiments, the antibody is an antagonistic PD-1 antibody or antibody fragment. In various embodiments, the antibody is an antagonistic PD-1 antibody comprising the variable domain sequences set forth in SEQ ID NOS: 138 and 139, SEQ ID NOS: 140 and 141, SEQ ID NOS: 142 and 143, SEQ ID NOS: 144 and 145, or SEQ ID NOS: 146 and 147. In various embodiments, the antibody is an antagonistic human PD-L1 antibody comprising the variable domain sequences set forth in SEQ ID NOS: 148 and 149. In various embodiments, the antibody is an antagonistic human CTLA-4 antibody comprising the variable domain sequences set forth in SEQ ID NOS: 150 and 151. In various embodiments, exemplary bifunctional IL-2 PD1 antibody fusion proteins are listed in Table 12.

### Bifunctional IL-2 Variant PD-1 Antibody Fusion Proteins

In various embodiments, immune checkpoint blocking antibodies that bypass the immunosuppressive effects in the tumor microenvironment or immune-stimulatory antibodies to potentiate existing responses are used to construct IL-2 antibody fusion proteins. The expression levels of negative immune checkpoints are particularly increased on tumor-antigen experienced exhausted T cells infiltrated in the tumor microenvironment. In various embodiments, tethering IL-2 variants to antibodies targeting immune checkpoints is expected to direct IL-2 to exhausted T cells and make tumor microenvironment immunologically hot. In various embodiments, Bifunctional IL-2 variant checkpoint inhibitor antibody fusion proteins can deliver IL-2 preferentially in cis to checkpoint inhibitor-expressing cells, such as tumor-antigen experienced exhausted T cells infiltrated in the tumor microenvironment, to facilitate selective signaling and enhance activity at the desired tumor site. In various embodiment, bifunctional IL-2 variant checkpoint inhibitor antibody fusion proteins provide synergy by removing the negative regulation and reinvigorating T cells in function and expanding Teff cell number to further enhance the immune system's activity against tumors.

In various embodiments, bifunctional IL-2 variant checkpoint inhibitor antibody fusion proteins reduce systemic exposure of IL-2 and off target toxicity. In various embodiments, the use of IL-2 variants with both reduced/abolished binding to IL-2Rα and attenuated/modulated IL-2Rβγ activity facilitate the establishment of stoichiometric balance between the cytokine IL-2 activity and antibody activity. Attenuated IL-2 activity variants with adequate antibody targeting or cis-activation at the exhausted Teff cells will allow optimal dosing and maintain function of each arm. Further, attenuated IL-2 activity variants fused with antibody is expected to minimize peripheral activation, reduce T cell AICD, mitigate antigen-sink, and promote tumor killing via the antibody targeting moiety to tumor and or immune cell site.

In various embodiments, the IL-2 variants of the present invention can be attached to checkpoint inhibitor that is an antibody, an antibody fragment, a protein, or a peptide targeting immune checkpoint modulators. In various embodiments, the immune checkpoint inhibitor is an antagonist PD-1 antibody. In various embodiments, the PD-1 antibody comprising the variable domain sequences set forth in SEQ ID NOS: 138 and 139, SEQ ID NOS: 140 and 141, SEQ ID NOS: 142 and 143, SEQ ID NOS: 144 and 145, or SEQ ID NOS: 146 and 147. In various embodiments, exemplary bifunctional IL-2 PD1 antibody fusion proteins are listed in Table 12.

### Linkers

In various embodiments, the heterologous protein is attached to the IL-2 variant by a linker and/or a hinge linker peptide. The linker or hinge linker may be an artificial sequence of between 5, 10, 15, 20, 30, 40 or more amino acids that are relatively free of secondary structure or display α-helical conformation.

Peptide linker provides covalent linkage and additional structural and/or spatial flexibility between protein domains. As known in the art, peptide linkers contain flexible amino acid residues, such as glycine and serine. In various embodiments, peptide linker may include 1-100 amino acids. In various embodiments, a spacer can contain motif of GGGSGGGS (SEQ ID NO: 18). In other embodiments, a linker can contain motif of GGGGS (SEQ ID NO: 21)n, wherein n is an integer from 1 to 10. In other embodiments, a linker can also contain amino acids other than glycine and serine. In another embodiment, a linker can contain other protein motifs, including but not limited to, sequences of α-helical conformation such as AEAAAKEAAAKEAAAKA (SEQ ID NO: 16). In various embodiments, linker length and composition can be tuned to optimize activity or developability, including but not limited to, expression level and aggregation propensity. In another embodiment, the peptide linker can be a simple chemical bond, e.g., an amide bond (e.g., by chemical conjugation of PEG).

Exemplary peptide linkers are provided in Table 6:

TABLE 6

| Linker sequence | SEQ ID NO: |
|---|---|
| GGGSGGGSGGGS | 11 |
| GGGS | 12 |
| GSSGGSGGSGGSG | 13 |
| GSSGT | 14 |
| GGGGSGGGGSGGGS | 15 |
| AEAAAKEAAAKEAAAKA | 16 |
| GGGGSGGGGSGGGGSGGGGS | 17 |
| GGGSGGGS | 18 |
| GSGST | 19 |
| GGSS | 20 |
| GGGGS | 21 |
| GGSG | 22 |
| SGGG | 23 |
| GSGS | 24 |
| GSGSGS | 25 |
| GSGSGSGS | 26 |
| GSGSGSGSGS | 27 |
| GSGSGSGSGSGS | 28 |
| GGGGSGGGGS | 29 |
| GGGGSGGGGSGGGGS | 30 |

### Polynucleotides

In another aspect, the present disclosure provides isolated nucleic acid molecules comprising a polynucleotide encoding IL-2, an IL-2 variant, an IL-2 fusion protein, or an IL-2 variant fusion protein of the present disclosure. The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Genomic DNA encoding IL-2 polypeptides is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. cDNA is obtained from libraries prepared from mRNA isolated from various tissues that express IL-2. The DNA molecules of the disclosure include full-length genes as well as polynucleotides and fragments thereof. The full-length gene may also include sequences encoding the N-terminal signal sequence. Such nucleic acids may be used, for example, in methods for making the novel IL-2 variants.

In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In various embodiments, the recombinant nucleic acids of the present disclosure may be operably linked to one or more regulatory nucleotide sequences in an expression construct. Regulatory sequences are art-recognized and are selected to direct expression of the IL-2 variant. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect of the present disclosure, the subject nucleic acid is provided in an expression vector comprising a nucleotide sequence encoding an IL-2 variant and operably linked to at least one regulatory sequence. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding an IL-2 variant. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered. An exemplary expression vector suitable for expression of IL-2 is the pDSRa, (described in WO 90/14363, herein incorporated by reference) and its derivatives, containing IL-2 polynucleotides, as well as any additional suitable vectors known in the art or described below.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant IL-2 polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In various embodiments, a vector will be designed for production of the subject IL-2 variants in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject IL-2 variants in cells propagated in culture, e.g., to produce proteins, including fusion proteins or variant proteins, for purification.

This present disclosure also pertains to a host cell transfected with a recombinant gene including a nucleotide sequence coding an amino acid sequence for one or more of the subject IL-2 variants. The host cell may be any prokaryotic or eukaryotic cell. For example, an IL-2 variant of the present disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject IL-2 variants. For example, a host cell transfected with an expression vector encoding an IL-2 variant can be cultured under appropriate conditions to allow expression of the IL-2 variant to occur. The IL-2 variant may be secreted and isolated from a mixture of cells and medium containing the IL-2 variant. Alternatively, the IL-2 variant may be retained cytoplasmically or in a membrane fraction and the cells harvested, lysed and the protein isolated. A cell culture includes host cells, media and other byproducts. Suitable media for cell culture is well known in the art.

The polypeptides and proteins of the present disclosure can be purified according to protein purification techniques are well known to those of skill in the art. These techniques involve, at one level, the crude fractionation of the proteinaceous and nonproteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

### Pharmaceutical Compositions

In another aspect, the present disclosure provides a pharmaceutical composition comprising the IL-2 variants, or IL-2 variant fusion proteins, in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired IL-2 polypeptide or IL-2 polypeptide fusion protein, in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a IL-2 variant), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. In various embodiments, pharmaceutical compositions may be formulated in nanoparticles, as slow release hydrogel, or incorporated into oncolytic viruses. Such nanoparticles methods include, e.g., encapsulation in nanoparticles composed of polymers with a hydrophobic backbone and hydrophilic branches as drug carriers, encapsulation in microparticles, insertion into liposomes in emulsions, and conjugation to other molecules. Examples of nanoparticles include mucoadhesive nanoparticles coated with chitosan and Carbopol (Takeuchi et al., Adv. Drug Deliv. Rev. 47(1):39-54, 2001) and nanoparticles containing charged combination polyesters, poly (2-sulfobutyl-vinyl alcohol) and poly (D,L-lactic-co-glycolic acid) (Jung et al., Eur. J. Pharm. Biopharm. 50(1):147-160, 2000). Albumin-based nanoparticle compositions have been developed as a drug delivery system for delivering hydrophobic drugs such as a taxane. See, for example, U.S. Pat. Nos. 5,916,596; 6,506,405; 6,749,868; 6,537,579; 7,820,788; and 7,923,536. Abraxane®, an albumin stabilized nanoparticle formulation of paclitaxel, was approved in the United States in 2005 and subsequently in various other countries for treating metastatic breast cancer.

Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.001 mg/kg to up to about 100 mg/kg or more, depending on the factors mentioned above. Polypeptide compositions may be preferably injected or administered intravenously. Long-acting pharmaceutical compositions may be administered every three to four days, every week, or biweekly depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, or intraperitoneal or intratumorally; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively, or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### Therapeutic Uses

In one aspect, the present disclosure provides for a method of treating cancer cells in a subject, comprising administering to said subject a therapeutically effective amount (either as monotherapy or in a combination therapy regimen) of an IL-2 variant, or IL-2 variant fusion proteins, of the present disclosure in pharmaceutically acceptable carrier, wherein such administration inhibits the growth and/or proliferation of a cancer cell. Specifically, an IL-2 variant, or IL-2 variant fusion protein, of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypophthalmic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to, bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, various lymphocytic leukemia, various myelogenous leukemia, and hairy cell leukemia. In various embodiments, the cancer will be a cancer with high expression of TGF-β family member, such as activin A, myostatin, TGF-β and GDF15, e.g., pancreatic cancer, gastric cancer, ovarian cancer, colorectal cancer, melanoma leukemia, lung cancer, prostate cancer, brain cancer, bladder cancer, and head-neck cancer.

“Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an EC₅₀. A dose can then be formulated in animal models to achieve a circulating plasma concentration range that includes the EC₅₀ as determined in cell culture. Such information can be used to determine useful doses more accurately in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of an IL-2 variant, or IL-2 variant fusion protein, of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

The dosing frequency of the administration of the IL-2 variant, or IL-2 variant fusion protein pharmaceutical composition depends on the nature of the therapy and the particular disease being treated. The subject can be treated at regular intervals, such as twice weekly, weekly or monthly, until a desired therapeutic result is achieved. Exemplary dosing frequencies include but are not limited to: once weekly without break; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

As used herein, the terms “co-administration”, “co-administered” and “in combination with”, referring to the a IL-2 variant, or IL-2 variant fusion protein, of the disclosure and one or more other therapeutic agents, is intended to mean, and does refer to and include the following: simultaneous administration of such combination of a IL-2 variant, or IL-2 variant fusion protein, of the disclosure and therapeutic agent(s) to a subject in need of treatment, when such components are formulated together into a single dosage form which releases said components at substantially the same time to said subject; substantially simultaneous administration of such combination of a IL-2 variant, or IL-2 variant fusion protein, of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of a IL-2 variant, or IL-2 variant fusion protein, of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of a IL-2 variant, or IL-2 variant fusion protein, of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present disclosure provides a method for treating cancer or cancer metastasis in a subject, comprising administering a therapeutically effective amount of the pharmaceutical compositions of the invention in combination with a second therapy, including, but not limited to immunotherapy, cytotoxic chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of the combination methods described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 24.0 mg/m², 250 mg/m², 260 mg/m², and 300 mg/m².

In various embodiments, the combination therapy methods of the present disclosure may further comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, SIRP, CD47, CD40, TIGIT and VISTA; treatment using bispecific T cell engaging antibodies (BiTE®) such as blinatumomab: treatment involving administration of biological response modifiers such as IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; wherein the combination therapy provides increased effector cell killing of tumor cells, i.e., a synergy exists between the IL-2 variants and the immunotherapy when co-administered.

In various embodiments, the combination therapy comprises administering an IL-2 variant and the second agent composition simultaneously, either in the same pharmaceutical composition or in separate pharmaceutical composition. In various embodiments, an IL-2 variant composition and the second agent composition are administered sequentially, i.e., an IL-2 variant composition is administered either prior to or after the administration of the second agent composition. In various embodiments, the administrations of an IL-2 variant composition and the second agent composition are concurrent, i.e., the administration period of an IL-2 variant composition and the second agent composition overlap with each other. In various embodiments, the administrations of an IL-2 variant composition and the second agent composition are non-concurrent. For example, in various embodiments, the administration of an IL-2 variant composition is terminated before the second agent composition is administered. In various embodiments, the administration second agent composition is terminated before an IL-2 variant composition is administered.

The following examples are offered to illustrate the disclosure more fully but are not construed as limiting the scope thereof.

### Example 1

### Construction and Production of IL-2 Fc Fusion Constructs

All genes were codon optimized for expression in mammalian cells, which were synthesized and subcloned into the recipient mammalian expression vector (GenScript). Protein expression is driven by an CMV promoter and a synthetic SV40 polyA signal sequence is present at the 3′ end of the CDS. A leader sequence has been engineered at the N-terminus of the constructs to ensure appropriate signaling and processing for secretion.

The constructs were produced by co-transfecting HEK293-F cells growing in suspension with the mammalian expression vectors using polyethylenimine (PEI, 25,000 MW linear, Polysciences). If there were two or more expression vectors, the vectors were transfected in a 1:1 ratio. For transfection, HEK293 cells were cultivated in serum free FreeStyle™ 293 Expression Medium (ThermoFisher). For production in 1000 ml shaking flasks (working volume 330 mL), HEK293 cells were seeded at a density of 0.8×10⁶ cells/ml 24 hours before transfection. A total of 330 μg of DNA expression vectors were mixed with 16.7 ml Opti-mem Medium (ThermoFisher). After addition of 0.33 mg PEI diluted in 16.7 ml Opti-mem Medium, the mixture was vortexed for 15 sec and subsequently incubated for 10 min at room temperature. The DNA/PEI solution was then added to the cells and incubated at 37° C. in an incubator with 8% 002. Sodium butyrate (Millipore Sigma) was added to the cells on day 4 at a final concentration of 2 mM to help sustain protein expression. After 6 days of cultivation, supernatant was collected for purification by centrifugation for 20 min at 2200 rpm. The solution was sterile filtered (0.22 μm filter, Corning). The secreted protein was purified from cell culture supernatants using Protein A affinity chromatography.

Alternatively, the constructs were produced in ExpiCHO cells (ThermoFisher) following manufacturer's instructions.

For affinity chromatography each supernatant was loaded on a HiTrap MabSelectSure column (CV=5 mL, GE Healthcare) equilibrated with 25 ml phosphate buffered saline, pH 7.2 (ThermoFisher). Unbound protein was removed by washing with 5 column volumes PBS, pH 7.2 and target protein was eluted with 25 mM sodium citrate, 25 mM sodium chloride, pH 3.2. Protein solution was neutralized by adding 3% of 1 M Tris pH 10.2. Ion exchange chromatography or mix-mode chromatography, including but not limited to CaptoMMC (GE Healthcare), ceramic hydroxyapatite, or ceramic fluoroapatite (Bio-Rad) was also utilized to polish the Protein A material as needed. Target protein was concentrated with an Amicon®Ultra-15 concentrator 10 KDa NMWC (Merck Millipore Ltd.)

The purity and molecular weight of the purified constructs were analyzed by SDS-PAGE with and in the absence of a reducing agent and staining with Coomassie (Imperial^{R} Stain). The NuPAGE® Pre-Cast gel system (4-12% or 8-16% Bis-Tris, ThermoFisher) was used according to the manufacturer's instructions. The protein concentration of purified protein sample was determined by measuring the UV absorbance at 280 nm (Nanodrop Spectrophotometer, ThermoFisher) divided by the molar extinction coefficient calculated on the basis of the amino acid sequence. The aggregate content of the constructs was analyzed on an Agilent 1200 high-performance liquid chromatography (HPLC) system. Samples were injected onto an AdvanceBio size-exclusion column (300 Å, 4.6×150 mm, 2.7 μm, LC column, Agilent) using 150 mM sodium phosphate, pH 7.0 as the mobile phase at 25° C.

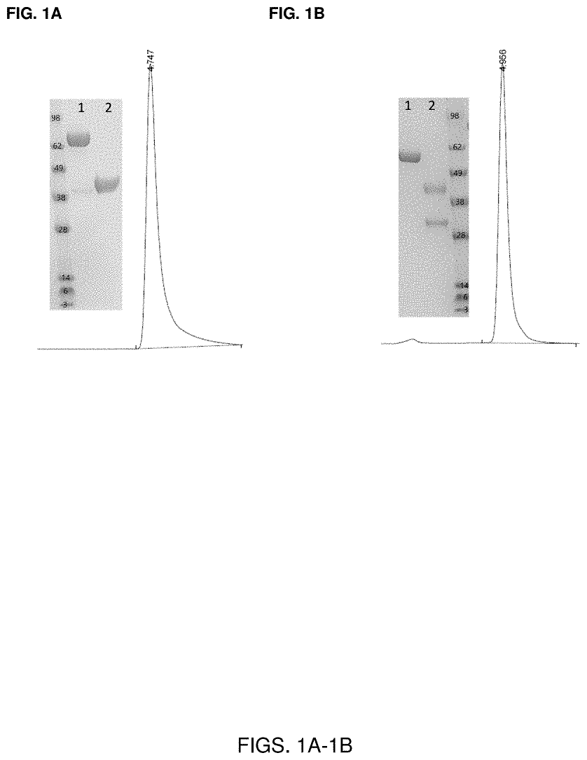

SDS-PAGE and size exclusion chromatogram analyses of protein A purified exemplary IL-2 variant Fc fusion constructs, P-0635 and P-0704, were illustrated in FIG. 1. P-0635 (SEQ ID NO: 85; FIG. 1A) and P-0704 (SEQ ID NOS: 96 and 10; FIG. 1B) share the same amino acid substitution P65R in IL-2. P-0635 comprises a bivalent IL-2 variant fused to homodimer Fc, while P-0704 comprises a monovalent IL-2 variant fused to knob-into-hole heterodimeric Fc. SDS-PAGE analysis demonstrated that both molecules exhibited high protein purity, and the samples run under reduced conditions (lane 2) showed expected MW for both the homodimer Fc chain in P-0635 and heterodimeric Fc chains of P-0704. Size exclusion chromatogram analysis showed that both molecules exhibited low aggregation propensity with less than 5% aggregation after initial protein A capture step.

### Example 2

### A Single Amino Acid Substitution in IL-2 Results in Universal Improvement in the Developability of the Fusion Compounds

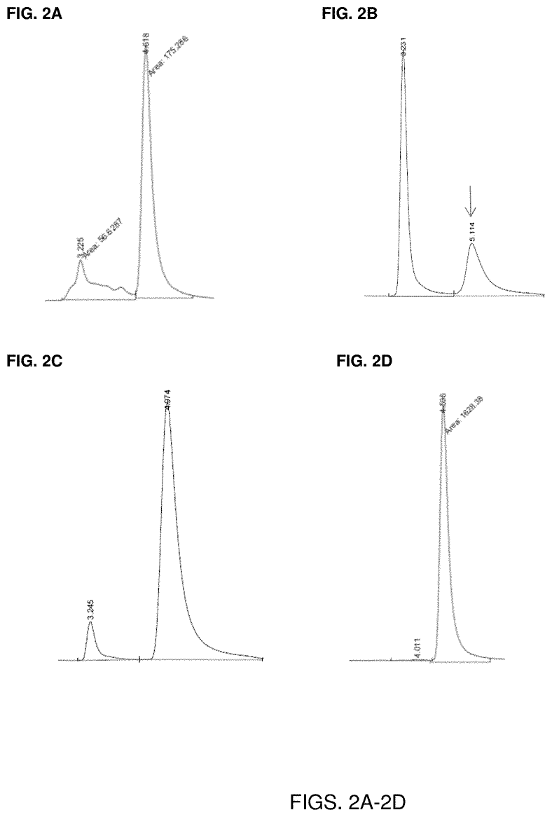

The engineering approach to find a combination of mutations that result in a variant protein with the desired biological properties encountered significant challenges when applied to IL-2. It is known in the field that naturally occurring IL-2 protein tends to be very unstable and is prone to aggregate. This was demonstrated in our experiments that the wild-type IL-2 Fc fusion protein (P-0250) expressed at a low level (around 3 mg/L transiently in HEK-293F cells) with high aggregation propensity, exemplified by SEC chromatogram depicted in FIG. 2A. The engineering efforts floundered as amino acid substitutions in IL-2 aimed at achieving the desired biological activity typically resulted in mutant proteins that are even less stable. A significant portion of IL-2 variants of the early phase of the current work expressed at extremely low level, and some variants were significantly more aggregation-prone, exemplified by SEC chromatogram of P-0318 (IL-2 D201/N881 Fc fusion) depicted in FIG. 2B. This is problematic for the manufacture and storage of a therapeutic agent.

It was also observed that the expression profiles and aggregation propensities of IL-2 variant fusions vary significantly among constructs with different mutation sites or mutants sharing the same mutation site but different residue substitutions. This observation is exemplified by P-0317 (IL-2 D201/N88R Fc fusion) and P-0318 (IL-2 D201/N881 Fc fusion). Both variant fusions share the same mutation sites at residues 20 and 88 and differ only by one amino acid and expressed at similarly low level. As can be seen in FIG. 2B, P-0318 is very aggregation-prone and contains 65% high-molecular weight species, which makes the expected peak as the minor species in the chromatogram and was marked with an arrow. In contrast, P-0317 is relatively pure with 7.5% aggregates (FIG. 2C). It would be deduced that N88R mutation may reduce aggregation propensity of the resulting fusion proteins. However, IL-2 with N88R single mutation, or D20T/N88R dual mutations, the resulting fusion proteins, P-0254 and P-0324, respectively, were aggregation-prone with 30-40% aggregates. So, the contributions of individual amino acid substitution to the protein stability seem to be context dependent.

The fact that amino acid substitutions to IL-2 typically result in less stable protein was further compounded by the unpredictable contributions of different residue substitutions to the protein stability. It is thus very desirable to find residue substitution(s) that can universally enhance protein developability, including improved stability, higher expression level, and lower aggregation propensity.

Amino acid substitutions at position 125 was originally aimed at tuned IL-2 selectivity as the residue is in immediate proximity to Q126, which is integral to the γc interaction. Naturally occurring IL-2 contains an unpaired cysteine at position 125, which was replaced by serine in Proleukin. IL-2 containing alanine substitution at position 125 is also widely used. As substitution of serine or alanine for cysteine at position 125 retained full biological activity, bulky charged or hydrophobic residues, including Glu, Lys, Try, His, and Iso, were introduced at position 125 aiming to interfere the interaction of Q126 with γc so as to achieve altered biological activity. All the resulting fusion molecules but the fusion molecule contains Iso125 (P-0531) expressed at too low level to be characterized. When compared to its S125 counterpart (P-0250), P-0531 expressed at a significantly higher level (29.5 mg/L vs 3.1 mg/L titer) with greatly reduced aggregation propensity (0.7% vs 25.7% aggregation). The impressive improvement in developability, especially on the product purity prompted us to evaluate whether such enhancement by isoleucine substitution at position 125 can be recapitulated in different mutational context.

S125I substitution was thus introduced into a number of IL-2 variant Fc fusion molecules. The constructs harboring Ile substitution at amino acid position 125 (125I) in IL-2 were expressed using the same vector and in the same culturing conditions as their Ser-125 counterparts and purified using MabSelectSure. The expression level in mg/L and purity assessed by SEC chromatography in aggregation % of exemplary molecules are summarized in Table 7. The two molecules in the same row of Table 7 share the same other amino acid substitution(s) and differ only at residue 125 with either serine or isoleucine. The SEC profile of P-0531 (SEQ ID NO: 68), the S125I equivalent of wild-type IL-2 Fc fusion, was further illustrated in FIG. 2D. It is clear from Table 7 that isoleucine substitution at position 125 resulted in 4 to 11-fold enhanced expression level and uniformly low aggregation propensity.

TABLE 7
The S125I substitution reduced aggregation and increased
expression of various IL-2 fusion proteins

| | | | | | expression |
|---|---|---|---|---|---|
| | Serine-125 | | Isoleucine-125 | | fold↑ |
| IL-2 | Aggre- | Expres- | Aggre- | Expres- | by S125I |
| Amino acid | gation | sion | gation | sion | substi- |
| substitution | % (SEC) | (mg/L) | % (SEC) | (mg/L) | tution |
| w/t | 25.7 | 3.1 | 0.7 | 29.5 | 9.6 |
| L19H | 21.4 | 7.7 | 0.6 | 36.7 | 4.8 |
| L19D | 32.6 | 2.6 | 0 | 13.6 | 5.2 |
| L19Y | 21.7 | 4.0 | 1.0 | 19.3 | 4.8 |
| D20T | 29.4 | 1.4 | 0.5 | 11.7 | 8.4 |
| D20E | 21.1 | 0.7 | 1.7 | 7.9 | 11.3 |
| L19H/Q126E | 23.7 | 7.3 | 0.7 | 26.6 | 3.6 |
| L19Y/Q126E | 33.8 | 6.7 | 0.8 | 23.5 | 3.5 |

It is evident from current invention that isoleucine at position 125 resulted in universal improvement in developability of the IL-2 fusion constructs. This finding is especially valuable as engineering of IL-2 for desired biological properties had been hindered by the fact that altering marginally stable wild-type IL-2 typically results in even less stale mutant proteins. The inherent challenges of IL-2 engineering can be mitigated by a single amino acid substitution at position 125 with isoleucine.

### Example 3

### Design of the IL-2 Constructs to Improved Selectivity for Effector T Cells and NK Cells

The main aspect of the present invention is to improve IL-2 selectivity relative to wild-type IL-2 for cells expressing IL-2Rβγ (but not IL-2Rα) over cells expressing IL-2Rαγ for cancer therapy. One approach used by the present inventors is to generate highly selective IL-2-Fc-fusion proteins through introduction of CD25-disrupting mutations into the cytokine component. Selection of CD25-disrupting mutations was based on inspection of the IL-2/IL-2R co-crystal structure (PDB code 2651). Multiple amino acid substitutions to one or two relevant residues at the interface with the IL-2 receptor a subunit, including R38, T41, F42, F44, E62, P65, E68, and Y107, were introduced aiming to reduce or abolish binding to IL-2Rα. These constructs also contained S125I mutation for significantly improved developability. Additionally, impairment of IL-2 variants in binding to IL-2Rα+ pulmonary endothelial cells is expected to prevent endothelial cell damage and significantly reduce VLS. Furthermore, impairment of CD25 binding is also expected to reduce CD25 antigen sink and enrich the cytokine occupancy to IL-2Rβγ-expressing cells and consequently enhanced in vivo response and tumor killing efficacy.

Table 3 summarizes the panel of IL-2 muteins expressed as C-terminal fusions to the Fc homodimer or Fc heterodimer. A panel of IL-2 variants (SEQ ID NOs: 31-66) harboring one or two amino acid substitutions to the residues at the interface with the IL-2 receptor a subunit were fused via a “GGGSGGGS” linker (SEQ ID NO: 18) to the C-terminus of either Fc homodimer as bivalent IL-2 fusions (SEQ ID Nos: 69-95) or Fc heterodimer as monovalent IL-2 fusions (SEQ ID Nos: 96-106).

### Example 4

### Impact of the IL-2 Mutations Introduced at the Interface with IL-2Rα on Binding to the Receptor Subunit α

A panel of IL-2 muteins was expressed as C-terminal fusions to the Fc homodimer of Fc heterodimer and screened for binding to IL-2Rα in enzyme-linked immunosorbent assay (ELISA). Briefly, IL-2Rα-ECD (SEQ ID NO: 5) was coated onto the wells of Nunc Maxisorp 96-well microplates at 0.1 μg/well. After overnight incubation at 4° C. and blocking with superblock (Thermo Fisher), 3-fold serial dilutions of IL-2 Fc fusion proteins starting at 100 nM were added to each well at 100 μI/well. Following one-hour incubation at room temperature, 100 μl/well of goat anti-human IgG Fc-HRP (1:5000 diluted in diluent) were added to each well and incubated at room temperature for 1 hour. Wells were thoroughly aspirated and washed three times with PBS/0.05% Tween-20 following each step. Finally, 100 μl TMB substrate was added to each well; the plate was developed at room temperature in the dark for 10 minutes, and 100 μl/well of stop solution (2N Sulfuric acid, Ricca Chemical) was added. Absorbance was determined at 450 nm and curves were fit using Prism software (Graph Pad).

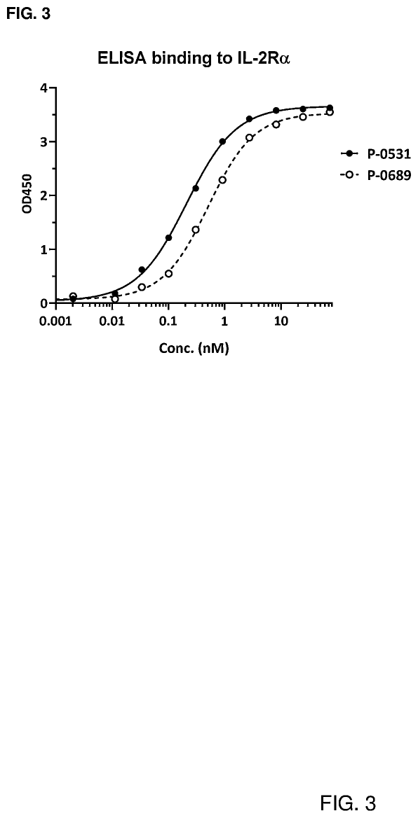

First, the S125I equivalent of wild-type IL-2 Fc fusion proteins, P-0531 and P-0689, were tested for CD25 binding. P-0531 comprises bivalent IL-2 moiety fused to Fc homodimer (SEQ ID NO: 68), and P-0689 ((SEQ ID NO: 107+10) is the monovalent counterpart of P-0531. As illustrated in FIG. 3, the 2-fold variation in binding EC₅₀ between P-0531 and P-0689 (0.21 nM and 0.51 nM, respectively) were consistent with the IL-2 valency difference.

Since all the targeted IL-2 residues, R38, T41, F42, F44, E62, P65, E68, and Y107, are at the interface with IL-2Rα and form either hydrogen bond/salt bridge or hydrophobic interactions with multiple IL-2Rα residues (Mathias Rickert, et al. (2005) Science 308, 1477-80), it was reasoned that amino acid substitutions at these sites are expected to disrupt interaction with IL-2Rα and resulted in IL-2 variants with reduced or abolished binding to IL-2Rα. However, the binding data revealed that the impact of different IL-2 mutations on IL-2Rα binding varied dramatically.

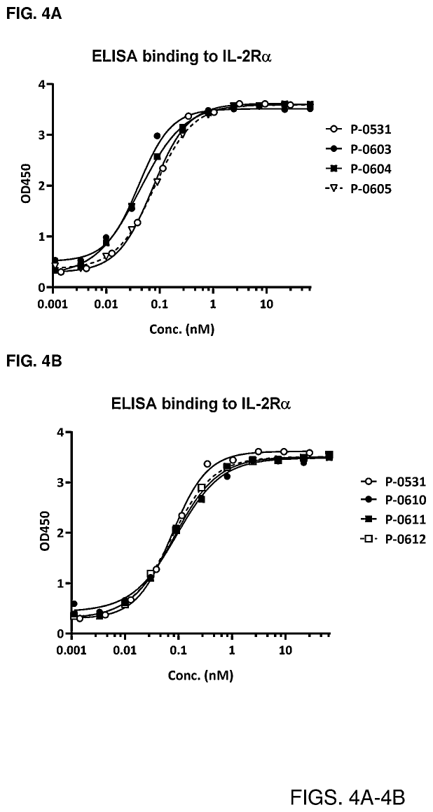

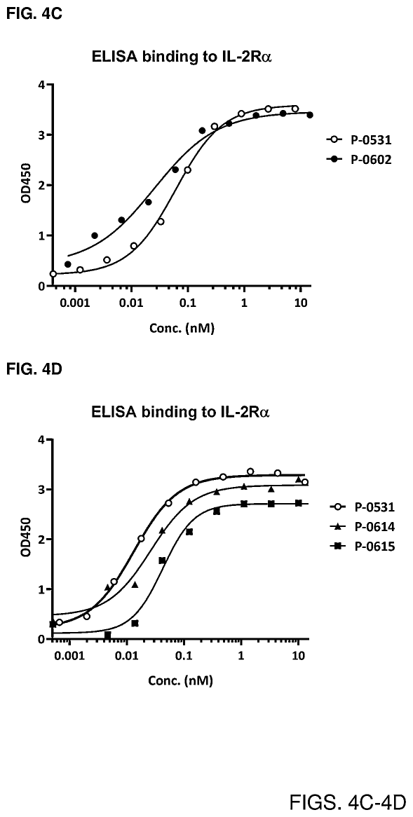

As illustrated in FIG. 4, IL-2 homodimer Fc fusions harboring various substitutions at position T41 (exemplified by P-0603, P-0604, and P-0605 in FIG. 4A) or Y107 (exemplified by P-0610, P-0611, and P-0612 in FIG. 4B) fully maintained the binding strength to IL-2Rα. The data suggested that residues T41 and Y107 are likely not functionally critical despite being at the interface of IL-2Rα and interacting with various IL-2Rα residues.

Residue R38 was implicated as an energetic hot spot for IL-2/IL-2Rα interaction, engaging in critical hydrogen bonds; multiple engineering efforts, e.g., Keith M. Heaton, et al, (1993) Cancer Res. 53. 2597-2602, and Peisheng Hu, et. al, (2003) Blood 101: 4853-4861, showed that a variety of substitutions at R38 resulted in disrupted interaction with IL-2Rα. Consequently, it was rather unexpected to observe that various mutations, exemplified by P-0602 (R38A), P-0614 (R38F), and P-0615 (R38G), resulted in no or only minimal reduction (up to 3-fold) in binding strength to IL-2Rα. The binding data are illustrated in FIGS. 4C-4D.

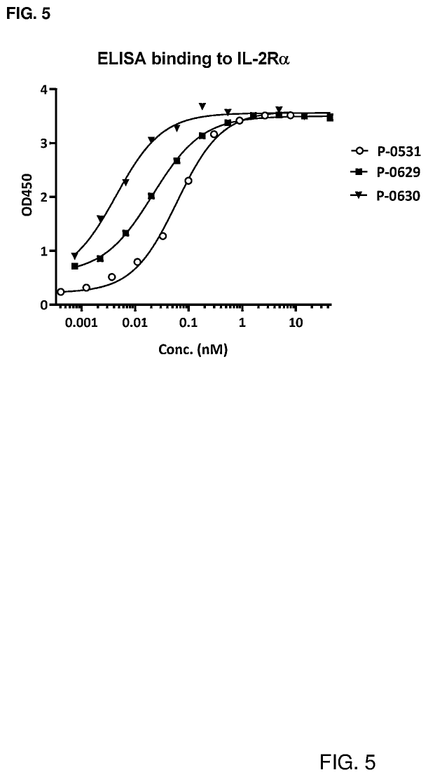

Likewise, residue E68 engages in multiple hydrogen bonds with IL-2Rα interface residues, but none of the substitutions at E68 of various amino acid properties, exemplified by E68A (P-0628), E68F (P-0629), E68H (P-0630), and E68L (P-0631), resulted in any reduction in binding to IL-2Rα. Interestingly, P-0629 and P-0630 actually demonstrated enhanced binding to IL-2Rα by 3- and 14-fold, respectively (FIG. 5).

In summary, replacement of IL-2 residues, T41, R38, E68, and Y107 generally did not disrupt IL-2Rα interaction and resulting IL-2 homodimer Fc fusions retained full or close to full binding to IL-2Rα. ELISA binding EC₅₀ of various IL-2 muteins normalized to that of P-0531 are summarized in Table 8.

TABLE 8
IL-2 residues whose replacements generally did not disrupt
IL-2Rα interaction and resulting IL-2 variants
retained full binding to IL-2Rα

| Protein | SEQ | IL-2 amino acid | Binding EC₅₀ |
|---|---|---|---|
| ID | ID NO: | substitutions | vs. P-0531 |
| P-0603 | 73 | T41A | 0.55 |
| P-0604 | 74 | T41G | 0.57 |
| P-0605 | 75 | T41V | 1.08 |
| P-0610 | 92 | Y107H | 1.24 |
| P-0611 | 93 | Y107L | 1.09 |
| P-0612 | 94 | Y107V | 1.00 |
| P-0614 | 70 | R38F | 0.42 |
| P-0615 | 71 | R38G | 2.0 |
| P-0602 | 72 | R38A | 3.23 |
| P-0628 | 86 | E68A | 1.0 |
| P-0629 | 87 | E68F | 0.35 |
| P-0630 | 88 | E68H | 0.073 |
| P-0631 | 89 | E68L | 1.0 |

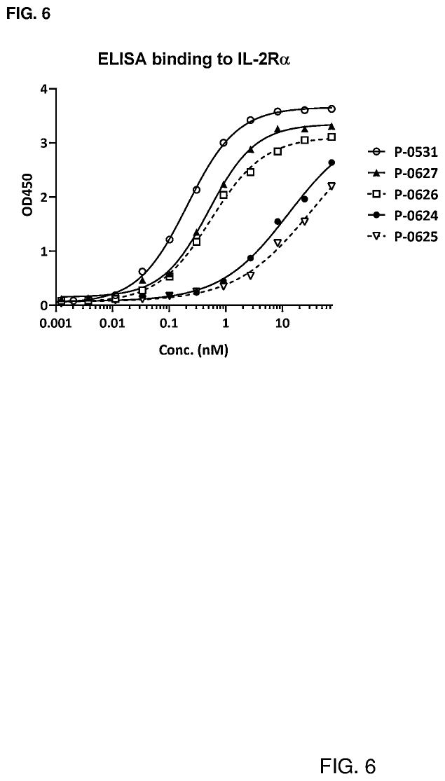

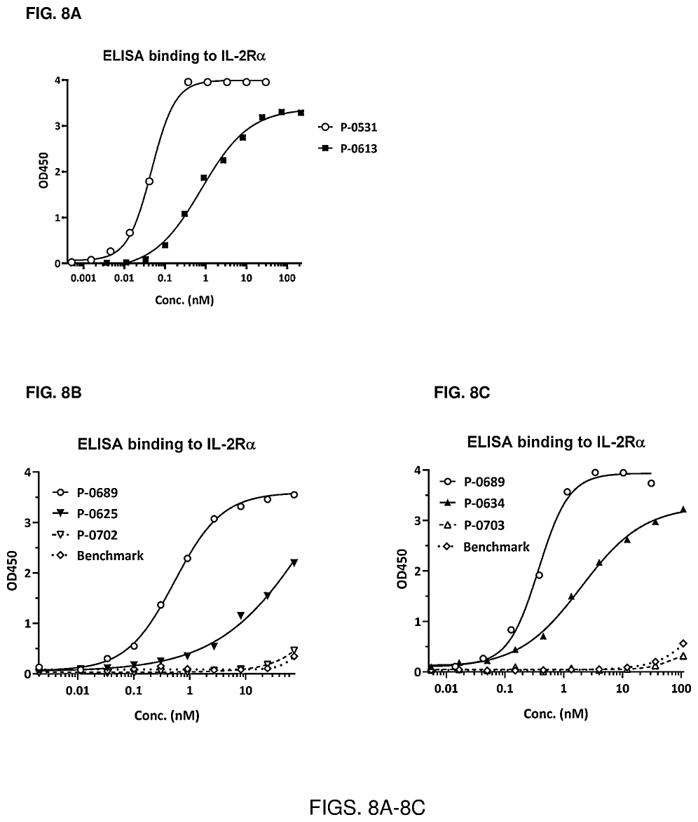

In contrast, amino acid substitutions at residue E62, exemplified by P-0624 (E62A), P-0625 (E62F), P-0626 (E62H), and P-0627 (E62L), all resulted in reduced binding to IL-2Rα, suggesting that E62 is indeed an energetic hot spot for IL-2/IL-2Rα interaction. As demonstrated in FIG. 6, while E62H and E62L substitutions only yielded in modest 2-3-fold reduction in binding to IL-2Rα, E62A and E62F mutations seem to produce drastic disruption in the interaction with this IL-2R subunit, resulted in 60- and 150-fold reduction in binding to IL-2Rα, respectively. Additionally, IL-2 F42A mutation (P-0613) was well documented in literature to disrupt interaction with receptor a, which was demonstrated in FIG. 8A, with a 15-fold reduction in binding to IL-2Rα.

In summary, F42 and E62 are IL-2 residues whose replacements generally disrupted IL-2Rα interaction and resulting IL-2 variants displayed reduced binding to IL-2Rα. ELISA binding EC₅₀ of various IL-2 muteins were normalized to that of P-0531 and shown in Table 9.

TABLE 9
IL-2 residues whose replacements generally disrupted IL-2Rα interaction
and resulting IL-2 variants had reduced binding to IL-2Rα

| Protein | SEQ | IL-2 amino acid | Binding EC₅₀ |
|---|---|---|---|
| ID | ID NO: | substitutions | vs. P-0531 |
| P-0613 | 69 | F42A | 15.6 |
| P-0624 | 78 | E62A | 60.5 |
| P-0625 | 79 | E62F | 151 |
| P-0626 | 80 | E62H | 2.57 |
| P-0627 | 81 | E62L | 2.38 |

### Example 5

### Amino Acid Substitutions at Residue P65 Yielded Unexpectedly Manifold Impact on Binding to Receptor Subunit α

IL-2 residue P65 engages Van der Waals interaction with a couple of critical IL-2Rα interface residues, including R36 and L42, but does not form salt bridges or hydrogen bonds with IL-2Rα. It was thus speculated that P65 substitutions may only result in modest disruption in interaction with this IL-2R subunit and likely cause mild impact on binding to IL-2Rα. However, the impacts of P65 substitutions on IL-2Rα interaction were in sharp contrast to the presumption and were unexpectedly manifold, including fully retain/enhance, reduce, or completely abolish binding to IL-2Rα.

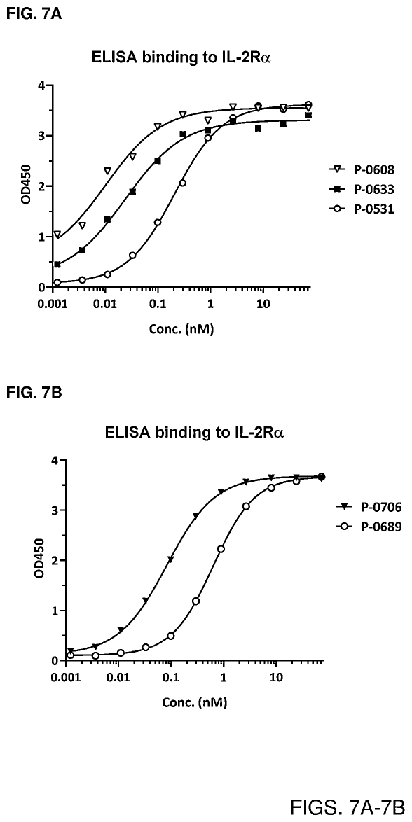

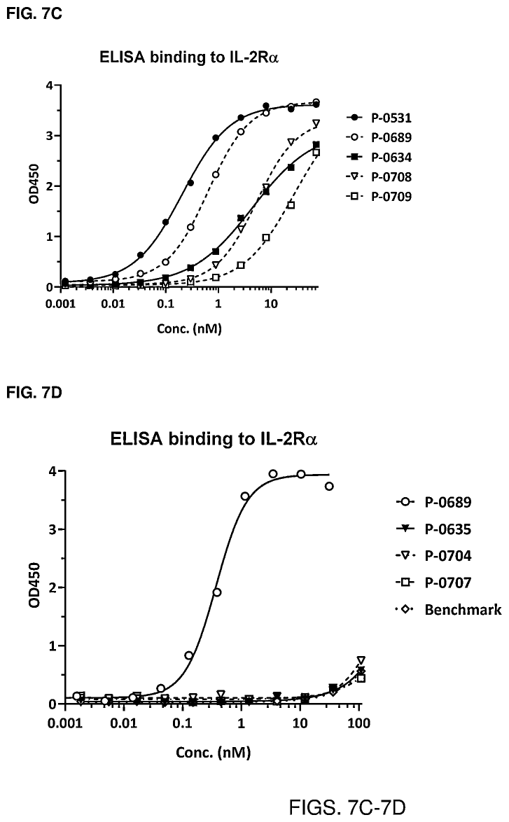

Multiple substitutions at P65, exemplified by P65G, P65E, P65A, P65H, P65N, P65Q, P65R, P65K, were introduced, and the resulting IL-2 muteins were expressed as C-terminal fusions to either Fc homodimer or Fc heterodimer. This panel of IL-2 muteins was subsequently screened in ELISA binding to CD25. The binding data were illustrated in FIG. 7, and ELISA binding EC₅₀ of IL-2 muteins normalized to that of either P-0531 or P-0689 to match each construct's valency were summarized in Table 10.

TABLE 10
Substitutions of P65 resulted in unexpectedly
manifold impact on IL-2Rα binding

| Protein | SEQ | IL-2 | IL-2 | Binding EC₅₀ |
|---|---|---|---|---|
| ID | ID NO: | substitutions | valency | vs. P-0531/P-0689 |
| P-0608 | 82 | P65G | Bivalent | 0.055 |
| P-0633 | 83 | P65E | Bivalent | 0.1 |
| P-0706 | 97 + 10 | P65A | Monovalent | 0.14 |
| P-0634 | 84 | P65H | Bivalent | 23 |
| P-0708 | 99 + 10 | P65N | Monovalent | 8.6 |
| P-0709 | 100 + 10 | P65Q | Monovalent | 43 |
| P-0635 | 85 | P65R | Bivalent | >500 |
| P-0704 | 96 + 10 | P65R | Monovalent | >500 |
| P-0707 | 98 + 10 | P65K | Monovalent | >500 |

As illustrated in FIGS. 7A and 7B, P65G (P-0608), P65E (P-0633), P65A (P-0706) mutations seemed not to produce any disruption in the interaction with IL-2Rα subunit; rather, the binding strength to IL-2Rα was enhanced by 18, 10, and, 10 fold, respectively when compare to their wild-type counterparts.

Another panel of IL-2 mutein Fc fusions, P-0634, P-0708, and P-0709, harbors P65 mutations that caused significant disruption in IL-2 interaction with IL-2Rα subunit. As illustrated in FIG. 7C and summarized in Table 9, P65N (P-0708) caused a modest 8.6-fold reduction in binding to IL-2Rα, while P65H (P-0634) and P65H (P-0709) substitutions resulted in more pronounced impact, which was demonstrated by a 23-fold and 43-fold reduction in IL-2Rα binding, respectively.

Yet another category of IL-2 P65 substitutions, P65R and P65K, seemed to engender drastic disruption in IL-2 and IL-R2Rα interaction and abolished binding of P-0635, P-0704, and P-0707 to the IL-2Rα (FIG. 7D). P-0635 and P-0704 are the bivalent and monovalent counterparts of IL-2 Fc fusion comprising P65R substitution, and P-0707 harbors P65K amino acid replacement. FIG. 7D illustrated that all the three IL-2 mutein Fc fusions showed minimal signal at IL-2Rα concentration as high as 100 nM, comparable to the Benchmark molecule, which harbors triple CD25-disrupting mutations F42A/Y45A/L72G that demonstrated to abolish binding (Christian Klein, et. al, OncoImmunology (2017), 6: 3, e1277306).

As summarized in FIGS. 7A-7C and Tables 9 and 10, substitutions of residue P65 resulted in unexpectedly manifold impact on IL-2Rα binding. Importantly, its substitution can either fully retain/enhance, reduce, or completely abolish binding of resulting IL-2 variants to IL-2Rα. As will be appreciated by those in the art, this level of activity variations resulting from changes to a single amino acid could not be predicted by structure-based mutagenesis approach. The complete abrogation of IL-2Rα binding was not expected or taught by the prior art either, as mutations to P65 only altered a limited part of the Van′ der Waal interaction surface.

### Example 6

### Amino Acid Substitution Combinations to Modulate IL-2 Binding to Receptor Subunit α

As will be appreciated by those in the art, the mutations disclosed in the current invention can be optionally and independently combined in any way to optimally modulate IL-2 binding to receptor subunit α. Here we demonstrate the design of IL-2 compounds incapable of binding to IL-2Rα by combining two IL-2Rα-disrupting amino acid substitutions.

P-0613 contains the F42A mutation, which resulted in a 15-fold reduction in binding to IL-2Rα (FIG. 8A), P-0625 and P-0634 harbor E62F and P65H substitutions had 150-fold and 23-fold reduced binding to IL-2Rα, respectively. Combination of F42A and E62F dual mutations in P-0702 and F42A and P65H dual mutations in P-0703 both resulted in abolished binding to IL-2Rα (FIGS. 8B and 8C). As expected, P-0766 comprising F42/E62A dual amino acid changes and P-0767 of F42A/E62H double substitutions were incapable of binding to IL-2Rα (data not shown).

In addition to serving as an effective way to design IL-2 muteins with abrogated binding to IL-2Rα, amino acid combinations also can be used to modulate the level of binding activity. One example illustrated here is P-0765, which combines one CD25-disrupting mutation F42A and one CD25-enhancing substitution, P65A, and there was a modest 6.8-fold reduction binding strength to IL-2Rα in comparison to its wild-type counterpart P-0689 (data not shown), which was in line with the combination of the individual mutations. ELISA binding EC₅₀ of IL-2 muteins normalized to that of P-0689 were summarized in Table 11.

TABLE 11
Impact of combinations of amino acid substitutions
at the CD25 interface on binding to IL-2Rα

| Protein | SEQ | IL-2 amino acid | Binding EC₅₀ |
|---|---|---|---|
| ID | ID NO: | substitutions | vs P-0689 |
| P-0702 | 101 + 10 | F42A/E62F | >500 |
| P-0766 | 102 + 10 | F42A/E62A | >500 |
| P-0767 | 103 + 10 | F42A/E62H | >500 |
| P-0703 | 104 + 10 | F42A/P65H | >500 |
| P-0705 | 105 + 10 | F42A/P65R | >500 |
| P-0765 | 106 + 10 | F42A/P65A | 6.8 |

In summary, amino acid substitution combination is a versatile approach to modulate IL-2 binding to receptor subunit α. It can achieve complete abrogation of IL-2Rα binding by combining two CD25-disrupting residues, or it may serve to modulate IL-2Rα binding with different attenuation levels.

### Example 7

### Modulation in IL-2Rα Binding Strength Correlates with IL-2 Potency in Stimulation Treg Cells in Ex Vivo Functional Assay

A panel of IL-2 variant Fc fusion proteins were subsequently examined for their ability to differentially stimulate STAT5 phosphorylation in CD4+ Treg cells in comparison to wild-type fusion P-0531 and benchmark molecule P-0551 (SEQ ID NO: 95). STAT5 is known to be involved in the downstream signaling cascade upon IL-2 binding to the transmembrane IL-2 receptors. The phosphorylation of STAT5 in lymphocyte subpopulations was measured using fresh human peripheral blood mononuclear cells (PBMC) and the forkhead transcription factor FOXP3 was used to identify the Treg population in FACS analysis.

Purified PBMC were starved in serum-free MACS buffer at 4° C. for 1 hour. 2×10⁵ PBMC were then treated with serial dilutions of test compounds for 30 min at 37° C. Cells were fixed and permeabilized with Foxp3/Transcription Factor Staining Buffer Set (EBIO) by incubating with 1×Foxp3 fixation/permeabilization working solution for 30 minutes and washing with 1× permeabilization buffer. Cells were additionally fixed with Cytofix buffer and permeabilized with Perm Buffer III (BD Biosciences) and then washed. After blocking Fc receptors by adding human TruStain FcX (1:50 dilution), cells were stained with a mixture of anti-CD25-PE, anti-FOXP3-APC, anti-pSTAT5-FITC, and anti-CD4-PerCP-Cy5.5 antibodies at concentrations recommended by the manufacturer for 45 minutes at room temperature. Cells were collected by centrifugation, washed, resuspended in FACS buffer, and analyzed by flow cytometry. The flow cytometry data was gated into CD4+/Foxp3+/CD25^{high} group for the Treg cell subsets. Data are expressed as a percent of pStat5 positive cells in gated population.

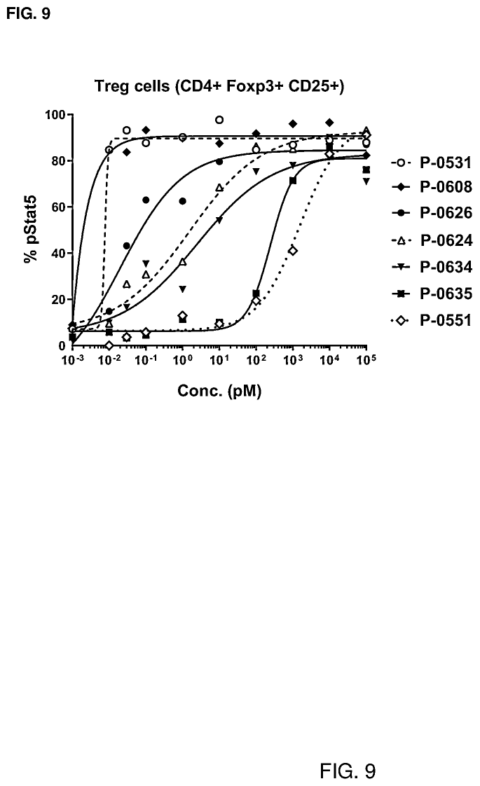

This panel of IL-2 variant Fc fusions contain amino acid substitutions that render either enhanced binding (P-0608), reduced binding (P-0626, P-0634, and P-0624), or abolished binding (P-0635) to IL-2Rα. Further, P-0626, P-0634, and P-0624 displayed different levels of attenuation in IL-2Rα binding strength; the reduction in binding was 2.6, 23, and 60-fold for P-0626, P-0634, and P-0624, respectively. The trend and level of modulation in IL-2Rα binding was reflected in the differential potencies of various IL-2 variant Fc fusions in stimulating STAT5 phosphorylation in CD4+ Treg cells (FIG. 9). P-0608 with enhanced binding to IL-2Rα correspondingly displayed a trend of higher potency than P-0531 in stimulating Treg STAT5 phosphorylation. P-0626, P-0624, and P-0634 all showed reduced pSTAT5 potency, consistent with their lower IL-2Rα binding strength. Their retained albeit lower binding to IL-2Rα resulted in Tregs still being more potently activated than P-0635 and benchmark P-0551, which were abolished of binding to IL-2Rα. P-0635 and P-0551 have comparable 5-log right-shift of potency in inducing pSTAT5 in Treg cells, and this low level of Treg signaling is likely resulted from activation of IL-Rβγ expressed on Treg cells. Consequently, the mutants are expected to achieve the desired property of activating Tregs at the concentration when CD8+ T and NK cells were also activated. It is striking to observe that the complete abrogation of IL-2Rα binding resulted in over 5 logs reduction in Treg potency (FIG. 9).

### Example 8

### The Effect of IL-2 Mutations Introduced at IL-2Rα Interface on the Interaction with IL-2Rβγ

To investigate whether the IL-2 mutations introduced at IL-2Rα interface would affect IL-2 interaction with IL-2Rβγ, binding to IL-2Rβγ was assessed in ELISA for the same panel of IL-2 variant Fc fusion proteins in Example 7.

Briefly, recombinant IL-2Rβγ heterodimer comprising IL-2Rβ ECD (SEQ ID NO: 109) fused to the N-terminus of an Fc hole chain (SEQ ID NO: 10) and γc ECD (SEQ ID NO: 110) fused to the N-terminus of an Fc knob chain (SEQ ID NO: 9) was coated onto the wells of Nunc Maxisorp 96-well microplates at 2 μg/well. After overnight incubation at 4° C. and blocking with 1% BSA, 3-fold serial dilutions of IL-2 Fc fusion proteins starting at 10 nM were added to each well at 100 μl/well. Following a one-hour incubation at room temperature, 100 μl/well of biotin mouse anti-human IL-2 clone B33-2 (BD Biosiences) at 0.5 μg/ml were added to each well and incubated at room temperature for 1 hour. Subsequently, 100 μl/well of streptavidin-HRP (1:5000 diluted in diluent) were added to each well and incubated at room temperature for 40 min. Wells were thoroughly aspirated and washed three times with PBS/0.05% Tween-20 following each step. Finally, 100 μl TMB substrate was added to each well; the plate was developed at room temperature in the dark for 10 minutes, and 100 μl/well of stop solution (2N Sulfuric acid, Ricca Chemical) was added. Absorbance was determined at 450 nm and curves were fit using Prism software (GraphPad).

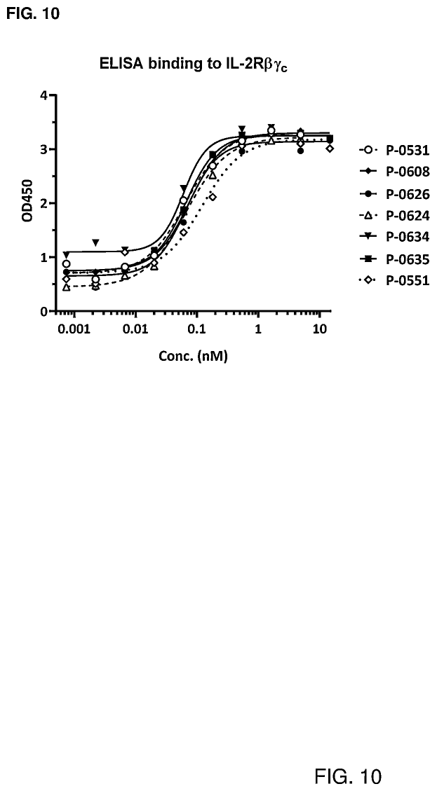

As shown in FIG. 10, compared to wild-type IL-2 fusion P-0531, the exemplary IL-2 variant Fc fusions, comprising mutations rendering either enhanced, reduced, or abolished binding to IL-2Rα, all displayed un-altered binding to IL-2Rβγ. The data confirmed that the tested IL-2 mutations introduced at IL-2Rα interface indeed only interfere with CD25 binding, and do not affect the interaction with IL-2Rβγ.

The panel of exemplary IL-2 variant Fc fusion proteins was further characterized for induction of Ki67 expression on human CD8+ T cells and NK cells by flow cytometry. Freshly isolated NK cells and CD8+ T cells express no or very low CD25 expression and the IL-2R signaling is mainly mediated via the intermediate affinity receptor subunits βγ. Ki67 is a nuclear protein served as a marker for cell proliferation.

Briefly, human PBMC were isolated by Ficoll-Hypaque centrifugation from the buffy coat of a healthy donor. Purified human PBMCs were treated with serial dilutions of IL-2 variant Fc fusion compounds and incubated at 37° C. for 5 days. On day 5, cells were washed once with FACS buffer (1% FBS/PBS) and first stained with Fc-blocker and surface marker antibodies, anti-human CD56-FITC, anti-human CD8-APC. After 30-minutes incubation and wash, cell pellets were fully resuspended by 200 μl/well of 1×Foxp3 fixation & permeabilization working solution and incubated for 30-minutes at room temperature in dark. After centrifugation, 200 μl of 1× permeabilization buffer were added to each well for another wash. Cell pellets were resuspended in permeabilization buffer with anti-human Ki67-PE (1:25 dilution). After 30-minutes incubation at room temperature, cells were collected and washed, resuspended in FACS buffer, and analyzed by flow cytometry. Data are expressed as % of Ki-67 positive cells in gated population.

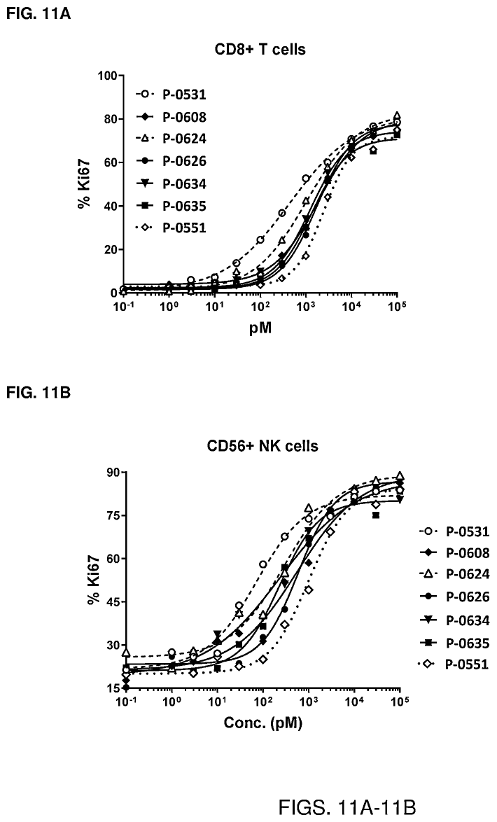

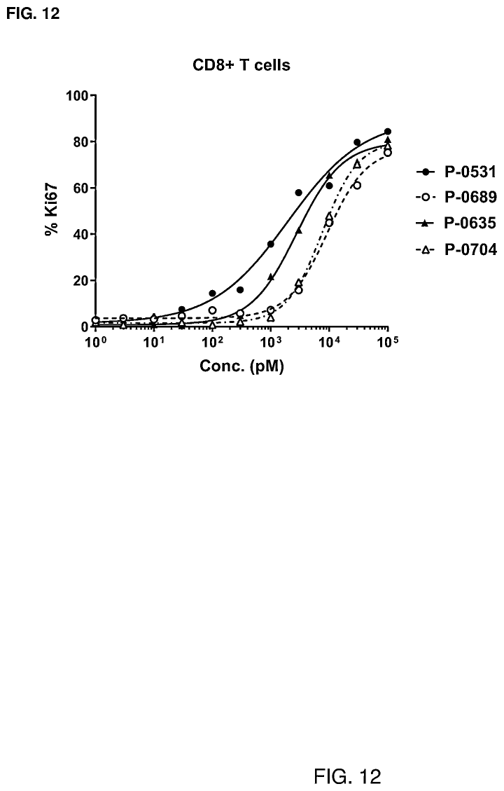

Dose-dependent increases of Ki-67 expression on CD8+ T cells and NK cells responding to IL-2 variant Fc fusion proteins in comparison to P-0531 and P-0551 were illustrated in FIGS. 11A and 11B. The introduction of mutations interfering with CD25 resulted in Fc fusion constructs with comparable potency to P-0531, the wild-type IL-2 bivalent fusion protein.

Further, P-0689 and P-0704, the monovalent counterparts of P-0531 and P-0635, respectively, were characterized for induction of Ki-67 expression on human CD8+ T cells. As illustrated in FIG. 11C, P-0689 (wild-type IL-2) and P-0704, harboring P65R mutation that abolished binding to IL-2Rα, showed equally potent dose-dependent increases of Ki-67 expression on CD8+ T cells. The combined ex vivo functional data further confirmed that IL-2 mutations introduced at IL-2Rα interface have minimal or no impact on the interaction with IL-2Rβγ. Moreover, potency variations between P-0531 and P-0689 and between P-0635 and P-0704 were consistent with their respective IL-2 valency differences.

### Example 9

### Introduction of IL-2Rβ or γc-Disrupting Substitutions to IL-2 Variants with Reduced Binding to IL-2Rα for Overall Potency Attenuation

Full IL-2 agonist could result in over-activation of the pathway and undesirable “on-target” “off-tissue” toxicity. It could be especially true for IL-2Rβγ selective full agonist; due to enhanced selectivity and reduced CD25 sink, IL-2Rβγ-selective full agonist can bolster dramatical in vivo responses of CD4+, CD8+ effector T and NK cells. As a result, acute toxicity may be observed with marked weight loss. In addition, overstimulation induced cell exhaustion or death may cause loss of response in vivo following repeated dosing. It was reasoned that lower overall potency may prevent pathway over-activation and reduce unwanted target sink; consequently, can potentially reduce toxicity and improve pharmacokinetics and pharmacodynamics. IL-2Rβγ-modulating substitutions to attenuate overall potency were thus incorporated to IL-2 variants with reduced/abolished binding to IL-2Rα for optimal activity. The attenuated binding affinity to IL-2Rβγ will also reduce receptor-mediated IL-2 internalization leading to slow but persistent receptor activation and durable pharmacodynamics than wild-type IL-2.

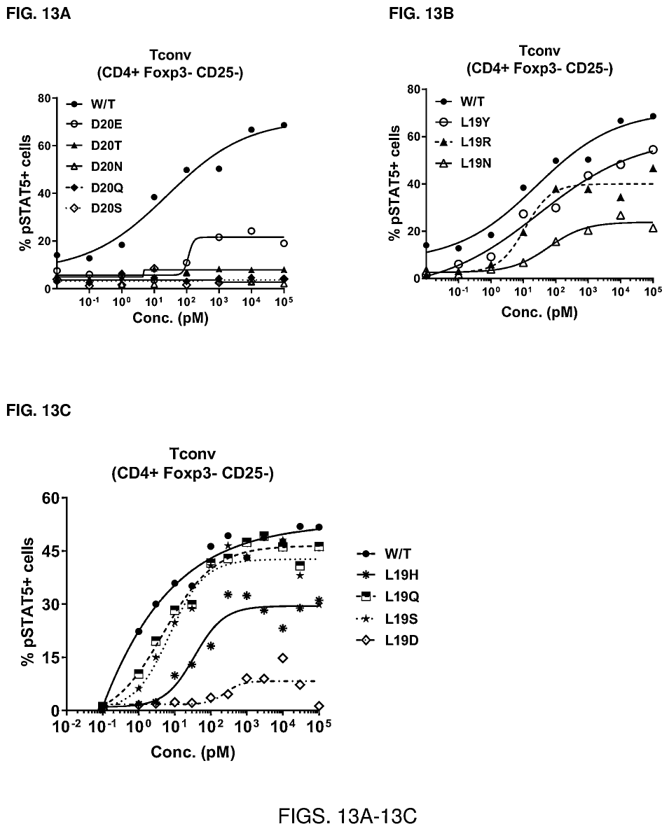

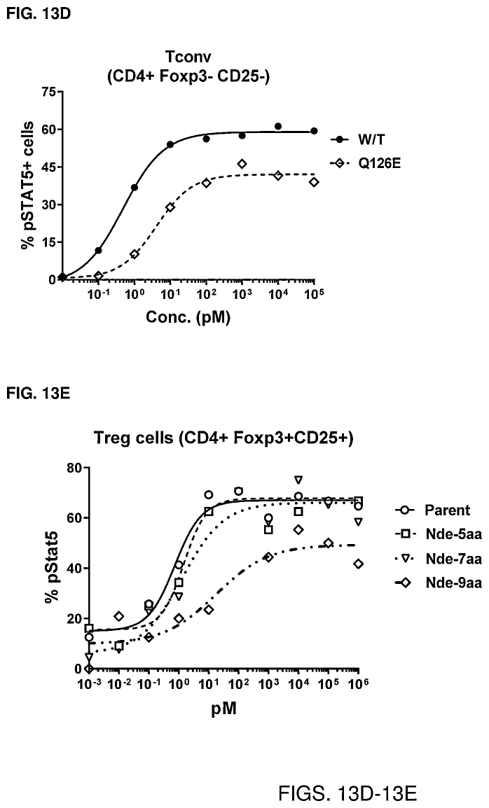

Selection of IL-2R6 or γc-disrupting mutations was based on inspection of the IL-2/IL-2R co-crystal structure (PDB code 2B51). Replacement of residues at or near the interface that make direct contact with IL-2Rβ or γc receptor subunits can resulted in reduced binding to IL-2Rβγ and thus modulate overall potency in activating the pathway. For example, D20 is engaged in an extensive network of hydrogen bonds to receptor subunit side chains at the IL-2Rβ interface. Similarly, N88 is an energetic hot spot for the IL-2/IL-2Rβ interaction, engaging in critical hydrogen bonds with the receptor chain. Q126 is integral to the γc interaction, However, amino acid substitutions at energy hot spot could resulted in substantially diminished activity rendering suboptimal potency, which was exemplified by various mutations at D20 position (D20E, D20T, D20N, D20Q, D20S) in FIG. 13A. All the mutations were introduced to IL-2 in P-0250 (SEQ ID: 67) and expressed as IL-2 variant Fc fusion proteins As depicted in FIG. 13A, majority of the mutations at D20 resulted in largely diminished or abolished activity in stimulating pSTAT5 expression in CD4+ Tconv cells, which express only the IL-2 Rβγ subunits. Similarly, mutations at position N88 also resulted in mostly abrogated activity in activation of CD4+ Tconv cells (data not shown)

Amino acid substitutions were thus introduced at position L19, a residue that only makes van der Waals interaction with IL-2Rβ, and the resulting mutants only modulate, not abrogate the IL-2 functional activity. FIGS. 13B and 13C shows that of IL-2 variants harboring various mutations at position 19 demonstrated a spectrum levels of potency in inducing STAT5 phosphorylation on CD4+ Tconv Cells. Compared to the wild type. L19Y, L19R, L19Q mutations resulted in mild activity reduction, while L19N and L19H moderately reduced the activity. For L19D, such activity was significantly impaired. Different levels of potency reduction by mutating position L19 facilitate activity fine tuning for optimal potency to reduce toxicity and improve pharmacokinetics and pharmacokinetics in vivo.

Further, IL-2 variants harboring amino acid changes at Q126, a residue that is integral to the γc interaction, were similarly made. The functional activity of IL-2 Q126E Fc fusion protein in inducing STAT5 phosphorylation on CD4+ Tconv cells is demonstrated in FIG. 13D. Compared to its wild-type counterpart, Q126E resulted in a modest activity reduction.

Additionally, as the amino acid at IL-2 N-terminus are mainly involved in the interaction with IL-2Rβγ, N-terminal amino acid deletion was considered as a different approach to modulate overall potency. Consequently, N-terminal deletion mutants (5, 7, 9, or 11-amino acid N-terminal deletions) build on an IL-2 variant harboring L19H/S125I/Q126E were constructed and assayed in human PBMC assay. As the parent molecule, IL-2 L19H/S125I/Q126E variant, retains full binding to IL-2Rα but diminished binding to IL-2Rβγ, so it can only be reliably assayed in Treg cells, which is still capable of dissecting the impact of mutations on overall potency. The Fc IL-2 variant comprising 11-aa deletion did not yield sufficient material for characterization. As depicted in FIG. 13E, while 5- and 7-aa deletions fully retained potency, 9-aa deletion resulted in a 25-fold activity impairment (18 pM vs. 0.74 pM). It is thus expected that various IL-2 variants of different potency could be further tuned for desired activity profile with amino acid deletions of 7, 8, 9, or 10 amino acids at the N-terminus.

IL-2Rβ-disrupting mutations L19H, L19Q, L19Y and γc-disrupting mutation Q126E was introduced to P-0704 yielding P-0731, P-0759, P-0761 and P-0732, respectively. P-0704 comprises P65R amino acid substitution that rendered complete loss of binding to IL-2Rα. P-0731, P-0759, P-0761 and P-0732 were assessed for binding to IL-2Rβγ in ELISA and for induction of Ki-67 expression on human CD8+ T cells, CD4+ T cells and NK cells by flow cytometry in comparison to P-0704.

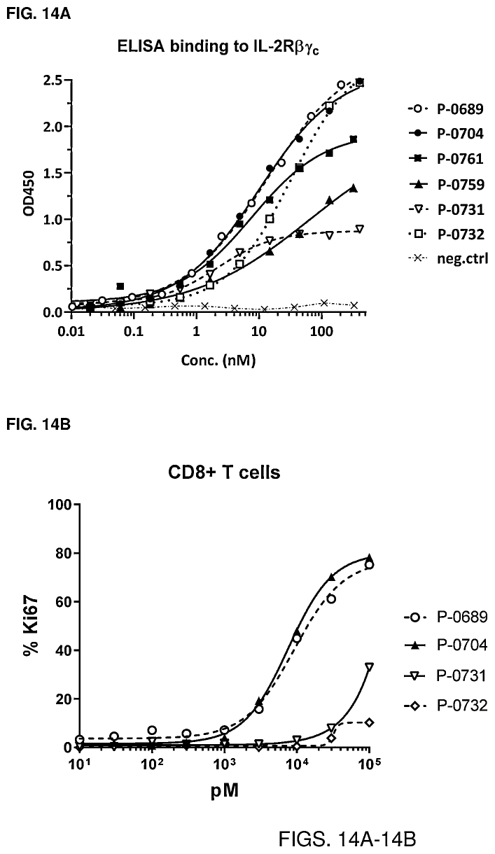

As shown in FIG. 14A, compared to P-0689 and P-0704, the exemplary IL-2 variant Fc fusions all displayed various level of reduced binding to IL-2Rβγ. As the binding of IL-2 to receptor subunit β or γ were weak and of high dissociation rate, the binding activity to each individual subunit could not be reliably assessed by ELISA (data not shown). However, the reduction in binding to IL-2Rβγ heterodimer was expected to be attributed by amino acid changes disrupting interaction with respective β or γ receptor subunit.

Potency reduction caused by IL-2Rβ-disrupting substitution L19H in P-0731 and by γc-disrupting mutation Q126E in P-0732 was assessed for the activity in inducing Ki67 expression on human CD8+ T cells in human PBMC. P-0689, S125I equivalent of wild-type IL-2 monomeric Fc fusion, and P-0704, which lost binding to IL-2Rα but fully retained affinity and functional activity for the dimeric IL-2Rβγ receptor, was included for comparison. As demonstrated in FIG. 14B, all the monomeric IL-2 Fc fusion proteins induced increased percentage of Ki-67 positive CD8+ T cells in a dose-dependent manner; P-0731 exhibited around 30-fold potency reduction compared to P-0704. P-0732 displayed the lowest potency with a greater than 100-fold weakened EC₅₀ in comparison to P-0704.

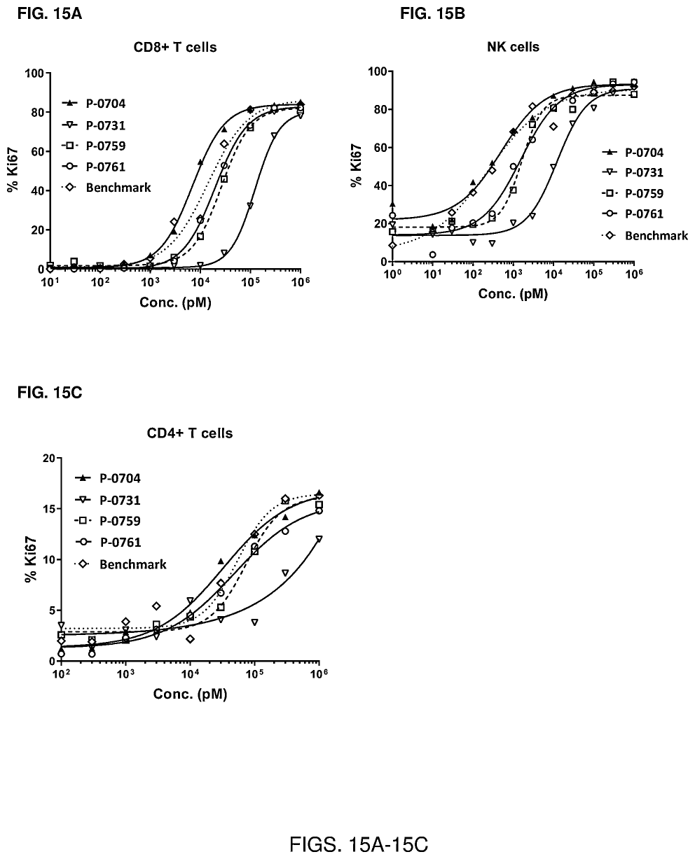

Dose-dependent increases in the proliferation of human CD8+ T cells, NK cells, and CD4+ T cells by P-0731, P-759, and P-0761 were illustrated in FIGS. 15A, 15B, and 15C, respectively. IL-2 variant Fc fusion proteins P-0731, P-0759, and P-0761 all contain IL-2Rβ-disrupting mutations at position L19 in addition to the IL-2Rα binding-abolishing substitution P65R in P-0704. Compared to P-0704, all variants showed expected potency reduction in the proliferation of human CD8+ T cells, NK cells, and CD4+ T cells. P-0759 (L19Q) and P-0761 (L19Y) showed a modest 3-5-fold potency reduction while L19H mutation in P-0731 resulted in a more profound 30-fold potency reduction. The level of potency attenuation by L19Q and L19H substitutions followed the same trend across all the cell subsets assessed and were consistent with the level of activity reduction in inducing pSTAT5 expression on CD4+ Tconv cells (FIGS. 13B and 13C) and the level of weakened binding to the recombinant IL-2Rβγ protein (FIG. 14A). Benchmark molecule displayed comparable but slightly lower potency in inducing cell proliferation in comparison to P-0704.

In summary, in addition to introduce CD25-disrupting substitution in IL-2 to curb undesirable expansion of immunosuppressive Tregs, IL-2Rβγ-disrupting substitutions or N-terminal deletions can be further incorporated to attenuate overall potency for optimal activity. Lower potency may prevent over-activation of the pathway and reduce unwanted target sink; consequently, can potentially reduce toxicity and improve pharmacokinetics and pharmacodynamics.

### Example 10

### Pharmacodynamic Effects of IL-2 Variant Fc Fusion Proteins in Mice Following a Single Injection

A time course of cell expansion of different lymphocyte subsets after treatment with P-0704 (SEQ ID NOS: 96 and 10), a C-terminal monovalent IL-2 variant Fc fusion protein with abolished binding to IL-2Rα, was conducted in Balb/C mice following a single injection. The effect on peripheral blood lymphocyte expansion was monitored over time. In addition, the immuno pharmacodynamics profiles of P-0704 were compared with those of P-0689 (SEQ ID NOS 107 and 10), the wild-type IL-2 counterpart.

Seven-week old female Balb/c mice were received from Charles River Laboratory and acclimated in house for at least 7 days before the study. Vehicle, and a single dose at 0.6 mg/kg of P-0704 and P-0689 was administered i.p. to mice on day 0. Blood samples were withdrawn on days 3 and 5 post injection. Each group included 4 mice.

Heparin-treated whole blood was used for immune phenotyping. After red blood cell lysis using BD pharm lysis buffer, total viable mononuclear blood cells were counted by trypan blue dead cells exclusion and proceeded to Ki67 intracellular staining. Cell pellets were fully resuspended by 200 ul/well of 1×Foxp3 fixation/permeabilization working solution and incubated for 30 minutes at room temperature in the dark. After centrifugation, 200 ul of 1× permeabilization buffer was added to each well for another wash. After blocking Fc-receptors with purified anti-mouse CD16/CD32 (1:50 dilution), cells were stained with APC-cy7 CD3, BV510 CD4, FITC Foxp3, PE Ki67, APC CD335, and Percpcy5.5 CD8 (1:50 dilution). After a 30-minute incubation, cells were collected and washed, resuspended in FACS buffer, and analyzed by flow cytometry.

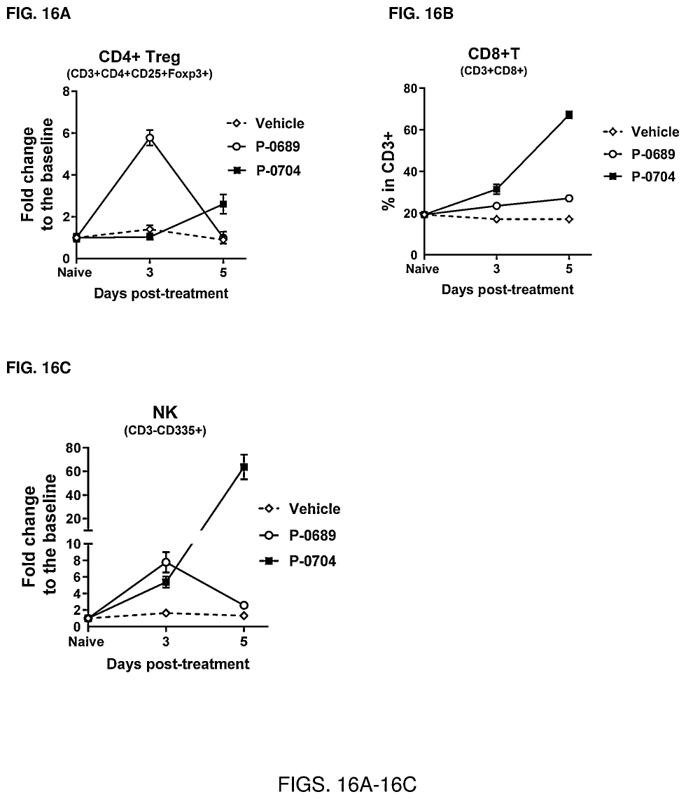

As shown in FIG. 16A, wild-type IL-2 in P-0689 resulted in strong expansion of Treg cells (6 fold increase in cell numbers) peaked on day 3, which is deemed undesirable for treatment of cancer, while P-0704 had no Treg expansion on day 3 and only minimally expanded Treg cells on Day 5. In contrast, P-0704 increased the percentage of CD8+ T cells in the total CD3+ lymphocyte population at Day 3 and continued enhanced CD8 population at Day 5 from 19% (baseline) to 67% (FIG. 16B). In contrary, CD8+ T cell expansion by P-0689 was minimal (FIG. 16B). For NK cells, a 5.4-fold cell number increase was observed on day 3, and the cell expansion continued and resulted in a 64-fold cell increase on day 5 by P-0704. P-0689 increased NK cell numbers by 7.8-fold on day 3, but the effect quickly diminished and returned to baseline on Day 5 (FIG. 16C).

In summary, P-0704 demonstrated nearly abolished Treg expansion and markedly enhanced CD8 and NK cell expansion, a sharply different cell expansion profile from P-0689. The observation is consistent with the drastic difference in the binding ability to IL-2Rα subunit and consequently Treg cell responsiveness. Additionally, as an IL-2Rβγ-selective full agonist, P-0704 can bolster dramatical in vivo responses of CD8+ effector T and NK cells due to enhanced selectivity and reduced CD25 sink.

### Example 11

### Construction, Expression, and Purification of IL-2-Antibody Fusion Proteins

In this example, various IL-2-antibody fusion proteins are prepared and evaluated. Tethering IL-2 variants to antibodies targeting immune checkpoints is expected to direct IL-2 to exhausted T cells and make tumor microenvironment immunologically hot. The strategy also reduces systemic exposure of IL-2 and off target toxicity. Bifunctional fusion protein of immune checkpoint inhibitors with IL-2 variants is also expected to provide synergy by removing the negative regulation and reinvigorating T cells in function and number. Immune checkpoint blocking antibody-cytokine fusion proteins are expected to further enhance the immune system's activity against tumors. The present inventors propose that the use of IL-2 variants with reduced/abolished binding to IL-2Rα and attenuated IL-2Rβγ activity is to facilitate the establishment of stoichiometric balance between the cytokine and antibody arms exhibiting dramatically different potency and molecular weights to allow optimal dosing and maintain function of each arm. Further, cytokine activity attenuation is expected to minimize peripheral activation, mitigate antigen-sink, and promote tumor targeting via the antibody arm.

For checkpoint inhibitor targets express on cytotoxic T cells or other lymphocyte subsets that also express IL-2Rβγ, such as PD-1, it is expected that IL-2 PD-1 antibody fusion proteins can deliver IL-2 variant preferentially in cis to PD-1+ cells, such as activated and exhausted CD8+ T in tumor microenvironment, to facilitate selective signaling.

Following this concept, various IL-2-antibody fusion proteins were constructed.

To prepare IL-2-antibody fusion proteins, the CH1-CH2-CH3 (antibody residue 118-447 based on EU numbering) domain of the heavy chains of the above listed antibodies were replaced with an IgG1 sequence set forth in SEQ ID NO: 162 which comprises L234A, L235A, G237A mutations to abolish binding to FcγR and C1q, but retain FcRn binding or PK. IL-2 variant peptide is fused via a peptide linker with sequences listed in Table 6 to the C-terminus of Fc domain. Alternatively, to express monovalent IL-2 variant, the CH1-CH2-CH3 domain of the heavy chains of the above listed antibodies were replaced with heterodimeric chains set forth in SEQ ID NO: 163-164. IL-2 variant peptide is fused via a peptide linker with sequences listed in Table 6 to the C-terminus of the knob-containing heterodimeric heavy chain engineered using the knob-into-holes technology. Half-life extension mutations, e.g., N434A, can be further incorporated into the homodimeric or heterodimer Fc chains. Exemplary IL-2 PD-1 antagonist antibody fusion proteins are listed in Table 12. Additionally, P-0844 is a benchmark IL-2 variant PD-1 antagonist antibody fusion protein comprising SEQ ID NOS:182-184.

TABLE 12
Exemplary IL-2 variant antagonist PD-1 antibody fusion proteins

| | IL-2 variant | IL-2-Antibody | | |
|---|---|---|---|---|
| IL-2 | polypeptide | fusion | IL-2 | Protein |
| mutations | SEQ ID NO: | SEQ ID NOS: | valency | ID |
| L19H/ | 111 | 165 + 141 | Dimer | P-0817 |
| P65R/S125I | | 176 + 175 + 141 | Monomer | P-0882 |
| P65R/S125I | 47 | 166 + 169 + 141 | Monomer | P-0803 |
| | | 177 + 175 + 141 | Monomer | P-0880 |
| P65Q/S125I | 51 | 167 + 169 + 141 | Monomer | P-0850 |
| L19Q/ | 117 | 168 + 169 + 141 | Monomer | P-0840 |
| P65Q/S125I | | 178 + 175 + 141 | Monomer | P-0841 |
| L19Q/ | 112 | 179 + 175 + 141 | Monomer | P-0885 |
| P65R/S125I | | | | |
| L19H/ | 114 | 180 + 175 + 141 | Monomer | P-0883 |
| P65Q/S125I | | | | |
| L19Q/ | 119 | 181 + 175 + 141 | Monomer | P-0884 |
| P65N/S125I | | | | |
| P65R/ | Amino acids | 189 + 175 + 141 | Monomer | P-0900 |
| S125I + 8- | 9-133 of | | | |
| aa N-terminal | SEQ ID | | | |
| deletion | NO: 47 | | | |
| P65R/ | Amino acids | 190 + 175 + 141 | Monomer | P-0901 |
| S125I + 9- | 10-133 of | | | |
| aa N-terminal | SEQ ID | | | |
| deletion | NO: 47 | | | |
| P65R/ | Amino acids | 191 + 175 + 141 | Monomer | P-0902 |
| S125I + 10- | 11-133 of | | | |
| aa N-terminal | SEQ ID | | | |
| deletion | NO: 47 | | | |

Gene synthesis, expression vector construction, and protein production, purification, and characterization were conducted following the same procedures detailed in Example 1.

Murine surrogate PD-1 IL-2 variant fusion proteins were produced analogously for use in vivo tumor models in immunocompetent mice. The surrogate anti-mouse PD-1 antibody comprises SEQ ID NOS: 185-187, which bearing Fc mutations for removal of effector function and for heterodimerization; IL-2 variant was fused to the C-terminus of anti-mouse PD-1 HC chain 2 (SEQ ID NO: 186) via a (G₄S)₃ linker (SEQ ID NO: 15). Table 13 lists the IL-2 variant in each exemplary murine surrogate PD1-IL-2 variant fusion protein:

TABLE 13
Exemplary murine surrogate PD-1 IL-2 variant fusion proteins

| Protein ID of IL-2 variant | IL-2 variant polypeptide |
|---|---|
| murine PD1 antibody fusion | SEQ ID NO: |
| P-0781 | 188 |
| P-0782 | 47 |
| P-0783 | 111 |
| P-0786 | 112 |
| P-0787 | 114 |
| P-0788 | 115 |
| P-0789 | 116 |
| P-0790 | 117 |
| P-0791 | 118 |
| p-0792 | 119 |
| P-0838 | 51 |
| P-0837 | 4 |

### Example 12

### IL-2 Variant Antibody Fusion Proteins Fully Retain IL-2 Potency and Activity Profiles in Ex Vivo Functional Assays

The surrogate mouse PD-1 antagonist antibody (SEQ ID NOS: 185-187) in current study does not cross-react with human antigen; consequently it was used as a non-functional antibody in human cells to assess the impact of the antibody fusion format on the potency and activity profile of IL-2 variants in stimulating and proliferating lymphocyte subsets.

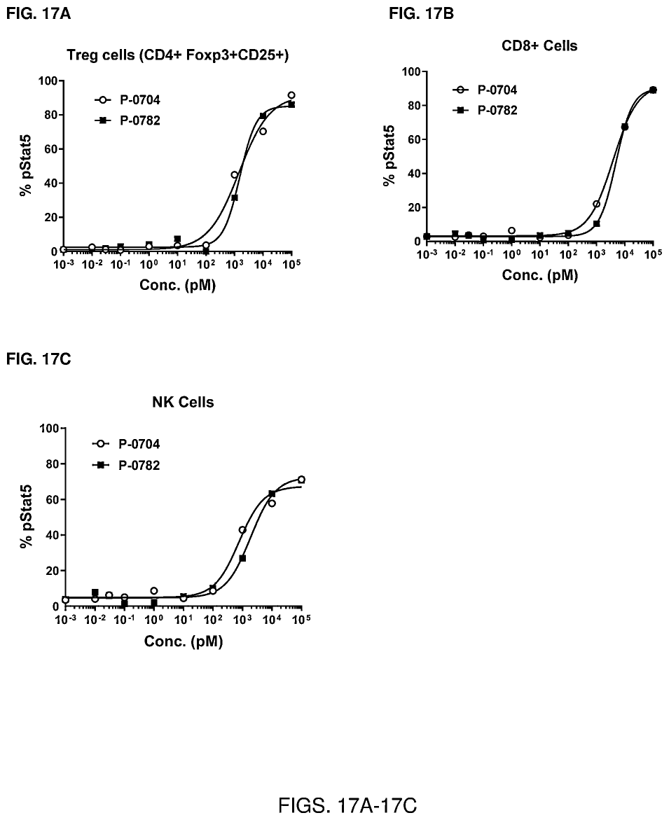

The impact of antibody fusion format was exemplified by P-0782 in comparison to its Fc fusion counterpart P-0704. Both P-0782 and P-0704 comprise monomeric IL-2 P65R variant linked to the C-terminus of heterodimeric Fc domain via a flexible (G₃S)₂ linker (SEQ ID NO: 18). The P65R substitution in IL-2 abolished binding to IL-2Rα (FIG. 7D). As depicted in FIGS. 17A-17C, P-0782 and P-0704 are equipotent in inducing dose-dependent STAT5 phosphorylation in CD4+ Treg cells (FIG. 17A), CD8+ T cells (FIG. 17B), and NK cells (FIG. 17C). The data confirmed that the IL-2 moiety fused to an antibody fully retained its activity as in its corresponding Fc fusion protein.

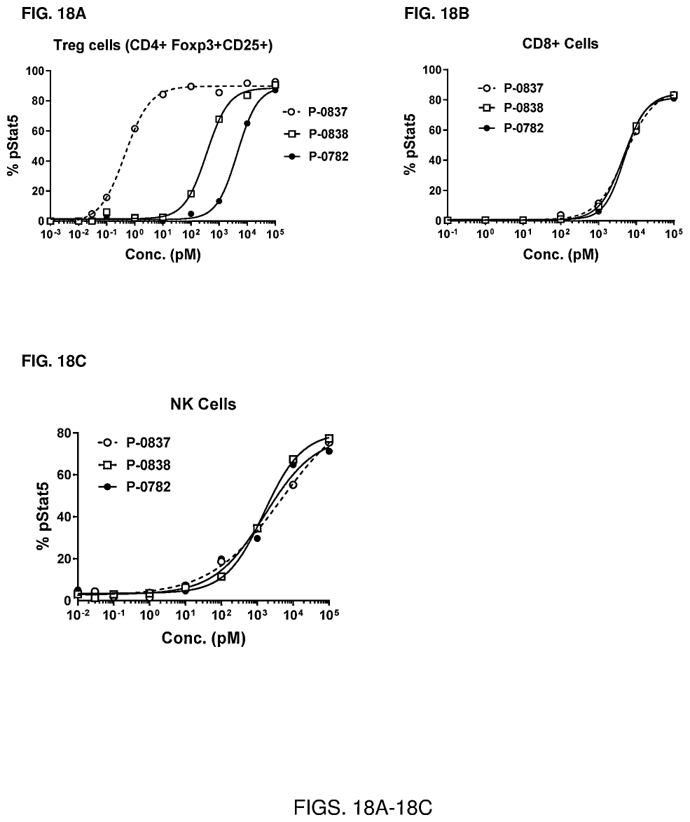

Further, three IL-2 variant mouse PD1 antibody fusion proteins, P-0837, P-0838, and P-0782, were compared for their activity in stimulating pSTAT5 in human PBMC. The IL-2 mutations P-0838 and P-0782 are P65Q and P65R, respectively. P-0837 comprises a wild-type IL-2 moiety (SEQ ID NO: 4). Compared to the wild-type, P65Q reduced IL-2Rα binding strength by 43 fold (Table 10) and P65R abolished binding to IL-2Rα. Corroborating findings of IL-2 Fc fusion molecules in earlier examples, FIGS. 18A-18C demonstrate that IL-2 mutations introduced at IL-2Rα interface indeed only interfere with CD25, and do not affect the interaction with IL-2Rβγ. As naïve CD8+ T and NK cells in human PBMC express no or very low levels of CD25, all the three molecules show identical potency in dose-dependent stimulation of pSTAT5 expression on these two lymphocyte subsets (FIGS. 18B and 18C). On the contrary, Treg cells constitutively express high levels of CD25, and consequently P-0838 and P-0782 showed dramatically reduced response in stimulating pSTAT5 expression in Treg cells than P-0837, the wild-type counterpart (FIG. 18A); EC₅₀s are 0.45 pM, 0.36 nM (800 fold weaker than P-0837), and 4.5 nM (10,000 fold weaker than P-0837) for P-0837, P-0838, and P-0782, respectively. Both CD25-binding reduced mutant P-0838 and CD25-binding abolished mutant P-0782 retained potency in stimulating CD8 and NK cells similarly as the wild-type counterpart. Additionally, abrogation of IL-2Rα binding in P-0782 resulted in EC₅₀ ratio of Treg/CD8 about 1, indicative of no preferential stimulation of Treg cells over cytotoxic effector cells (EC₅₀ 4.5 nM for Tregs vs 4.6 nM for CD8+ T cells). The presence, albeit significantly weakened IL-2Rα binding in P-0838, renders about 13-fold enhanced pSTAT5 responsiveness for Treg than CD8+ T cells (0.36 nM for Tregs vs 4.6 nM for CD8+ T cells).

The potency-attenuated IL-2 variants with IL-2Rβ-disrupting mutations L19Q or L19H in addition to reduced binding to IL-2a were also assessed in ex vivo functional assays for IL-2 antibody fusion format. Compared to P-0782, P-0786 comprises one additional L19Q substitution and P-0783 contains L19H. The Fc counterparts of P-0782, P-0786, and P-0783 are P-0704, P-0759, and P-0731, respectively.

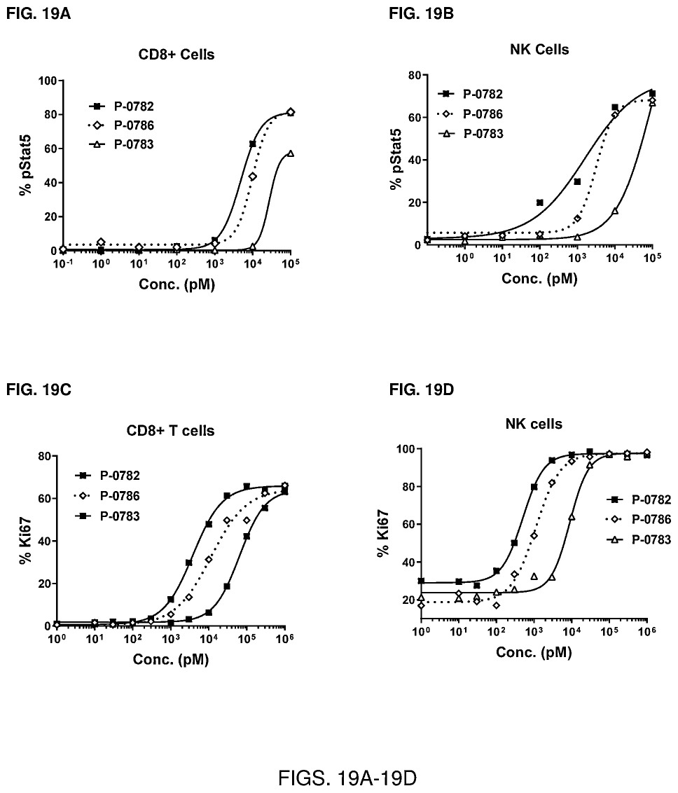

Induction of STAT5 phosphorylation by P-0782, P-0786, and P-0783 in a dose-dependent manner on human CD8+ T cells, and NK cells were illustrated in FIGS. 19A and 19B, respectively; dose-dependent increases in the proliferation of the same lymphocyte subsets were depicted in FIGS. 19C and 19D, respectively. Compared to P-0782, P-0786 showed a modest 2-3-fold potency reduction in inducing STAT5 phosphorylation on CD8+ T cells (FIG. 19A) and NK cells (FIG. 19B), while L19H mutation in P-0783 resulted in a more profound 20-30-fold potency reduction (FIGS. 19A and 19B). Similar level of potency attenuation was observed for dose dependent increases in Ki67 on CD8+ T cells (FIG. 19C) and NK cells (FIG. 19D). The level of potency attenuation in the antibody fusion proteins, P-0782, P-0786, and P-0783, followed the same trend as their corresponding Fc fusion proteins P-0704, P-0759 and P-0731, respectively (FIGS. 15A and 15B).

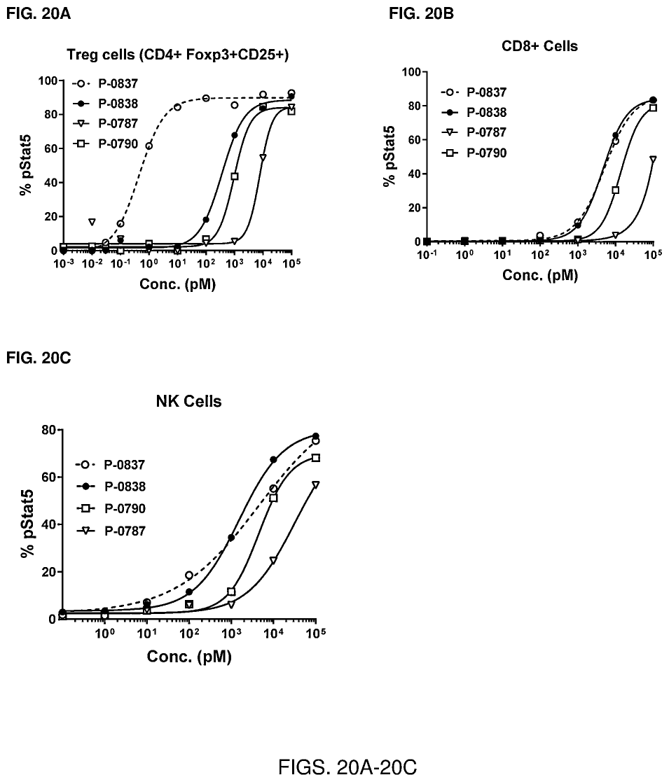

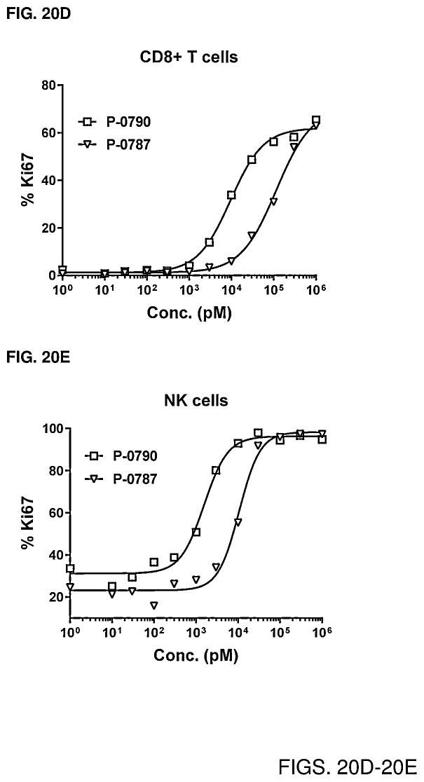

Potency-attenuation by IL-2Rβ-disrupt mutations were also evaluated in the context of P65Q mutation in IL-2 antibody fusion format. L19Q and L19H were introduced to P-0838 to make P-0790 and P-0787, respectively. FIGS. 20A, 20B and 20C display their activity in stimulating STAT5 phosphorylation on Treg, CD8+ T, and NK cells. FIGS. 20D and 20E shows the dose-dependent increases in the proliferation marker Ki67 on CD8+ T cells and NK cells. The levels of potency attenuation follow the same trend as observed for IL-Rα-abolishing substitution P65R-based Ab fusions.

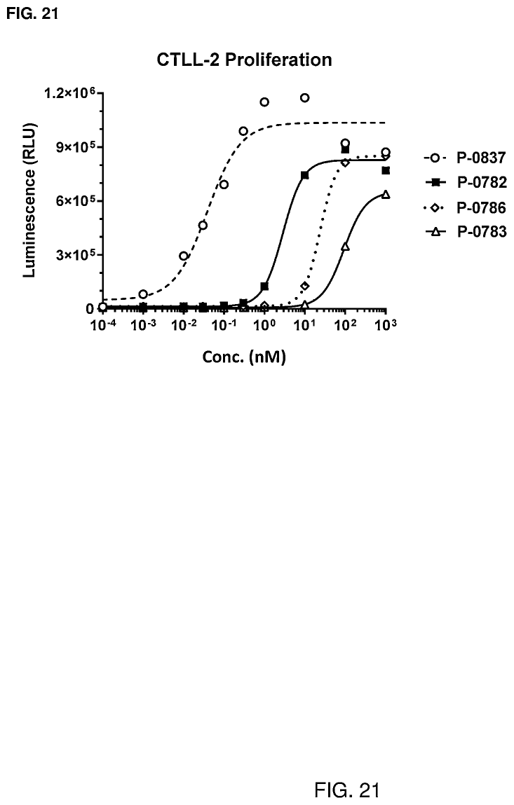

P-0782, P-0786, and P-0783 were further assessed for CTLL-2 proliferation activity along with P-0837, which comprises a S125I equivalent wild-type IL-2. CTLL-2 cells are C57BL/6 mouse-derived cytotoxic T cells expressing α, β, and γ receptor subunits. Briefly, CTLL2 cells were harvested, washed, and re-suspended in medium (RPMI1640, 10% FCS, 2 mM Glutamine) without IL-2 and incubated for two hours (IL-2 starvation). After starvation, 50 μl of CTLL-2 cells re-suspended at 50,000/ml in fresh medium without IL-2 were transferred to a 96-well U-bottom plate. Fifty μl of serially diluted IL-2 antibody fusion was added to wells to make a final volume of 100 μl/well. Samples were incubated for 2 days and proliferation was assessed using CellTiter-Glo according to manufacturer's instructions and luminescence signals were measured. As depicted in FIG. 21, levels of potency attenuation by L19Q and L19h also maintained in mouse cells. P-0837, comprising wild-type IL-2, demonstrated significant growth advantage over P-0782 due to the expression of IL-Ra subunit on CTLL-2 cells, resembling Treg cells.

In summary, IL-2 variants in surrogate mouse PD-1 antibody fusion protein format fully retained the potency and activity profiles as seen in their Fc fusion equivalents in ex vivo functional assays.

### Example 13

### In Vitro Characterization of IL-2 Variant Human PD-1 Antibody Fusion Proteins

P-0795 is a human PD-1 antagonist antibody comprising SEQ ID NO: 140 as the heavy chain and SEQ ID NO: 141 as the light chain. P-0803 (SEQ ID NOS: 166, 169 and 141) is an immunoconjugate of P-0795 with an IL-2 variant fused to the C-terminus of the knob-containing heterodimeric heavy chain. The IL-2 variant in P-0803 comprises an IL-2Rα binding-abolished mutation P65R and a developability-improving substitution 51251. The function of the antibody arm in the antibody fusion protein exemplified by P-0803 was assayed for both direct binding and ligand competitive inhibition in ELISA format.

For direct binding, the same ELISA protocol in Example 4 was followed using huPD-1-His as the coating antigen. For ligand (PD-L1) competitive inhibition ELISA, similar ELISA protocol was used with some modifications. Briefly, plate was coated with 0.2 μg/well of human PD1-Fc protein at 4° C. for overnight. After washing and blocking with 2% BSA, biotinylated Human PDL1-Fc at 0.5 μg/mL was mixed with serially-diluted P-0795 or P-0803 at 1:1 (v/v); 100 μL mixture was added to each well and incubate at 37° C. for 1 hour. Streptavidin-HRP was added as the secondary antibody.

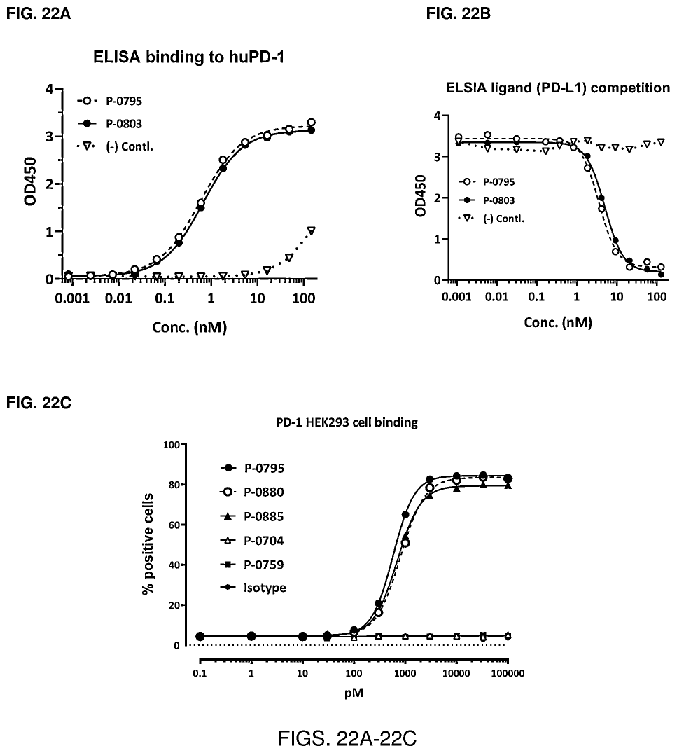

As depicted in FIG. 22A, P-0803 and P-0795 had identical binding strength to PD-1 (EC₅₀=0.6 nM). P-0803 also equally potent as P-0795 in blocking the binding of human PD-1 to PD-L1 immobilized on a surface (IC₅₀=2.1 nM; FIG. 22B). The data collectively confirmed that the antibody arm in the IL-2 antibody fusion is fully functional.

Similarly, IL-2 variant human PD-1 antibody IL-2 showed similar binding as the parent antibody to PD1 expressed on cell surface analyzed by FACS analysis (FIG. 22C). P-0795 is an antagonist human PD-1 antibody, and both P-0880 and P-0885 comprise monovalent IL-2 attached to the C-terminal of P-0795 via a (G₄S)₃ linker. P-0880 contains P65R/S125I substitution while P-0885 comprises L19Q/P65R/S125I mutations. P-0704 and P-0759 are the Fc fusion counterparts of P-0880 and P-0885, respectively. Due to the lack of PD-1-targeting arm, P-0704 and P-0759 did not bind to PD-1-expressing cells as expected.

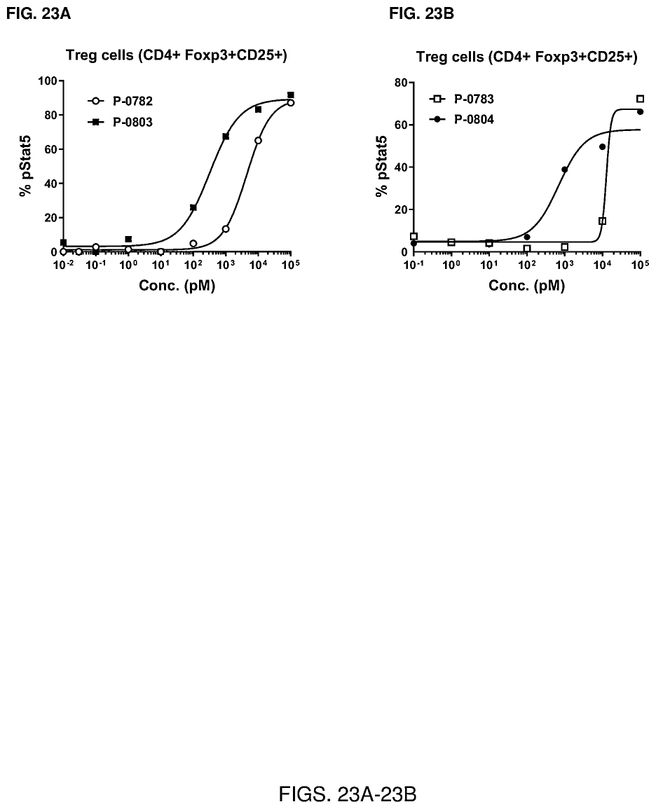

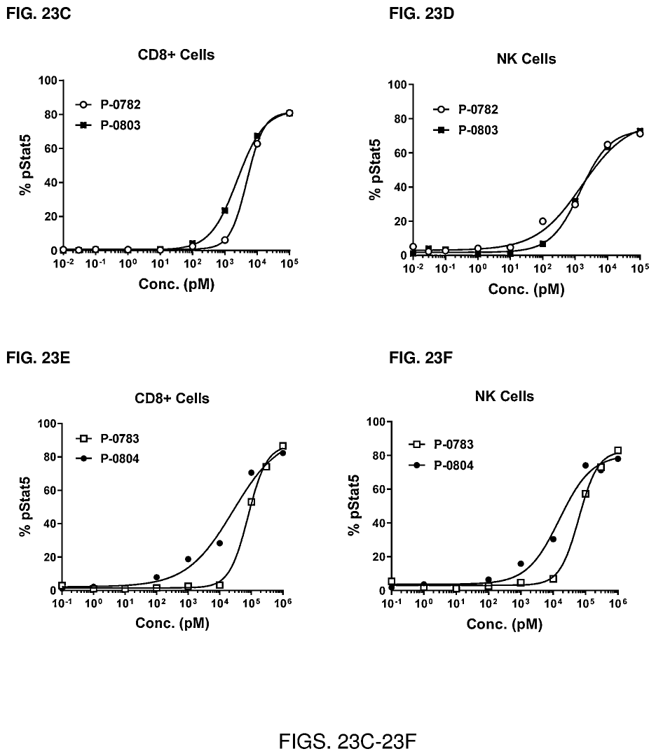

As PD-1 binds to the check point inhibitor PD-1, it is expected that the immunoconjugate can deliver IL-2 variant preferentially in cis to PD-1+ cells, such as activated and exhausted CD8+ T in tumor microenvironment, to facilitate selective signaling. In PBMC from healthy person, naive CD8+ T cells and NK cells are generally PD-1 negative while Tregs express low constitutive levels of PD-1. Consequently, it was observed that IL-2 huPD-1 Ab fusion proteins, P-0803 and P-0804, were over 15-fold more potent in stimulating pSTAT5 in PD-1 positive T cells than their non-PD-1 targeting equivalents, P-0782 and P-0783, respectively (FIGS. 23A and 23B); whereas, potency differences were minimal or mild on PD-1 negative cells (FIGS. 23C-23F). The huPD-1 Ab fusion proteins also showed a trend of increased potency compared to the non-PD-1 targeted counterparts in naïve non-activated CD8 and NK cells (FIGS. 23C-23F).

It is expected that higher PD-1 expression levels on T cells will more likely be targeted by the antibody fusion proteins to achieve selective signaling. Consequently, in the tumor microenvironment, IL-2 PD-1 antibody fusion proteins will preferentially bind to Teff vs. Tregs.

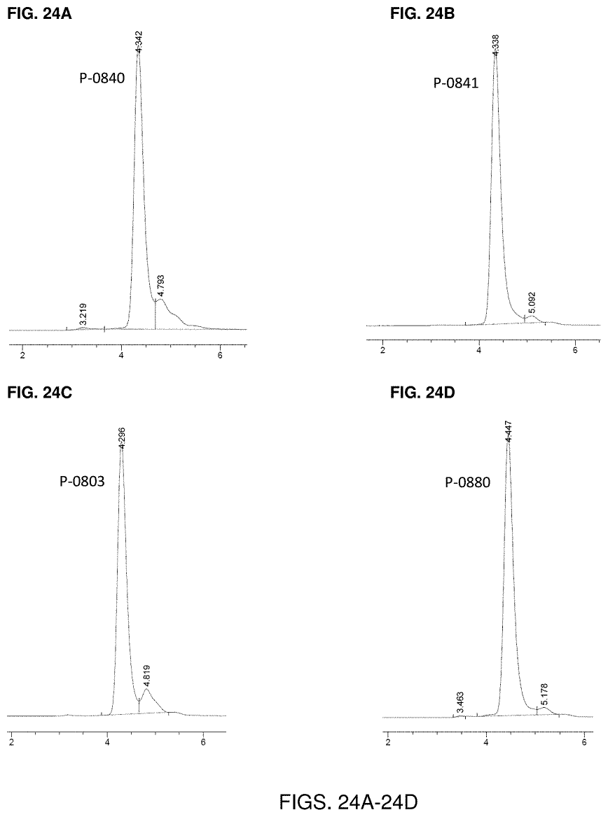

Further, the impact of the length of the linker connecting the antibody knob heavy chain and IL-2 variant on protein expression profile and activity was explored. P-0840 (SEQ ID NOS: 168, 169 and 141) and P-0841 (SEQ ID NOS:178, 175, and 141), are two IL-2 P-0795 fusion proteins that only differ in the linker length. P-0840 comprises a (G₃S)₂ linker (SEQ ID NO: 18) while P-0841 has a (G₄S)₃ linker (SEQ ID NO: 15). As shown in FIGS. 24A and 24B, Protein A purified P-0841 resulted from ExpiCHO transient expression displayed significantly less low molecular weight impurities than P-0840 yielded from identical production and purification processes (16% vs. 3%). Similar difference in impurity content was observed for P-0803 and P-0880 (SEQ ID NOS: 177, 175, and 141) (11% vs. 2.7%; FIGS. 24C and 24D), whose linkers are (G₃S)₂ and (G₄S)₃, respectively with otherwise identical sequences.

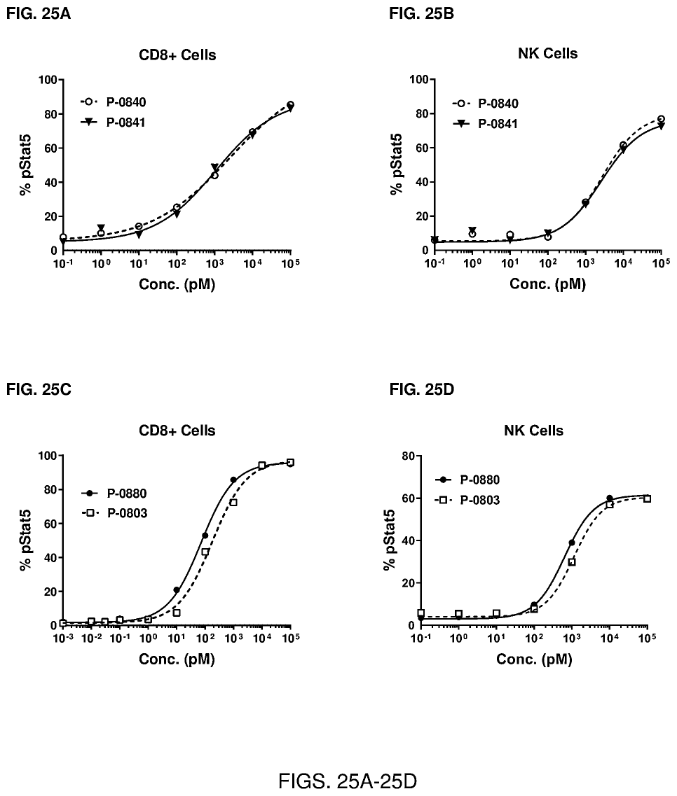

While a slightly longer linker in P-0841 and P-0880 resulted in improved purity compared to their respective shorter linker-containing counterparts, the impact on the biological activity of the IL-2 moiety were either minimal or marginally enhanced, exemplified by pSTAT5 stimulation potency on cytotoxic lymphocytes (FIG. 25). With the beneficial impact on the developability profile of fusion proteins, a longer linker without causing other negative impact is preferred over a shorter one.

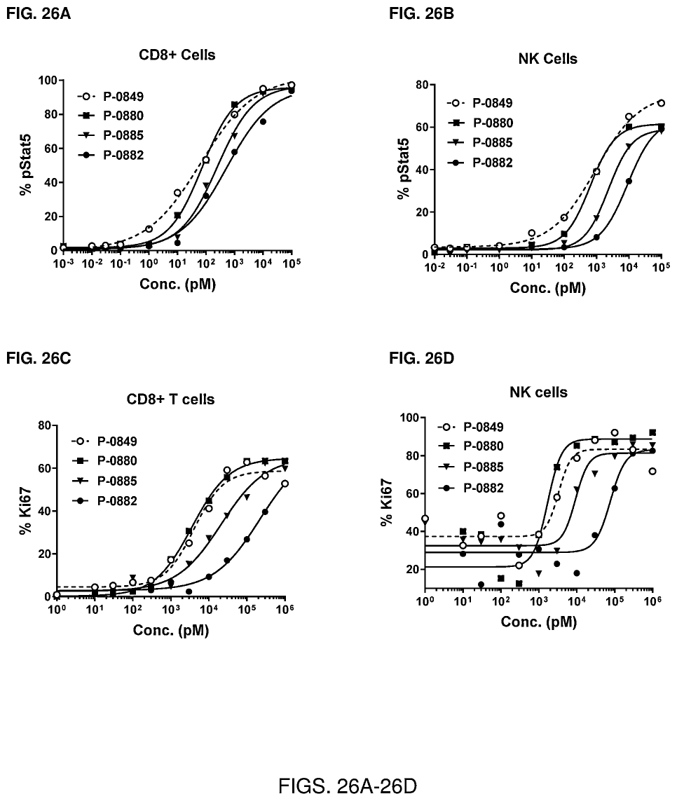

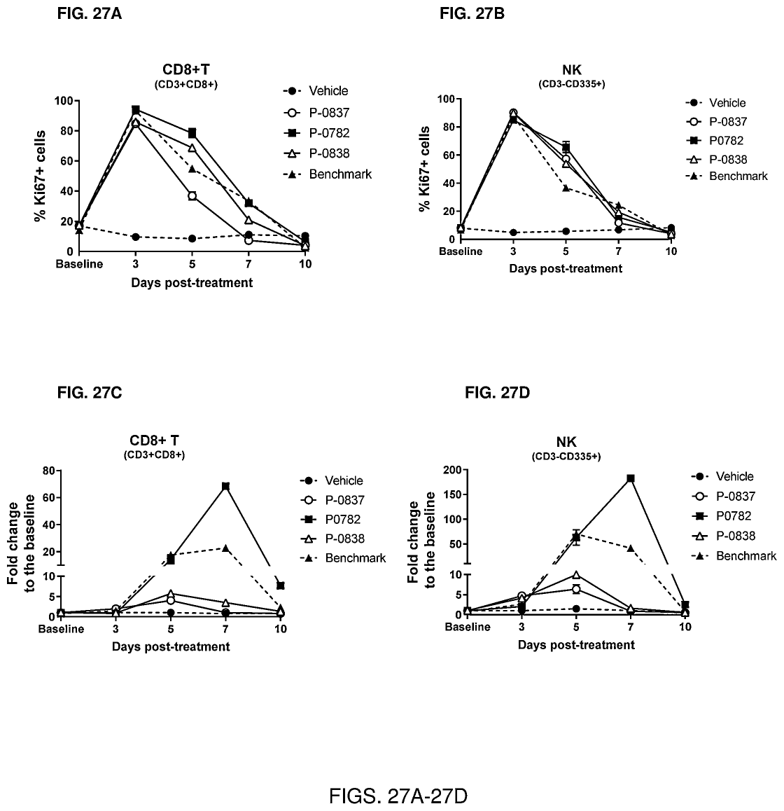

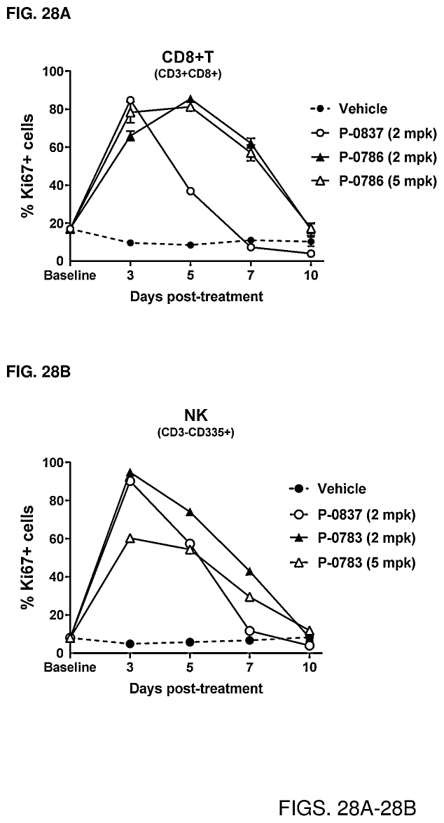

A few P-0795 fusion proteins with IL-2 variants fused to the C-terminus of the knob-containing heterodimeric heavy chain via (G₄S)₃ linker, P-0880, P-0882 (SEQ ID NOS: 176, 175 and 141), and P-0885 (SEQ ID NOS: 179, 175 and 141), were constructed. The binding to cell surface expressed PD-1 were not altered in IL-2 variant huPD-1 antibody fusion proteins with the longer linker compared to hPD1 antibody alone as shown in FIG. 22C. They were further tested in ex vivo functional assays to investigate IL-2 potency in stimulating pSTAT5 and inducing Ki67 expression in both CD8+ T cells and NK cells (FIG. 26). The three constructs all comprise IL-2Rα abolishing mutation P65R, while P-0882 and P-0885 contain additional L19H and L19Q mutations, respectively, to modulate the overall potency. P-0849, the wild-type IL-2 counterpart, was included in the assays for comparison. The ex vivo functional activities were summarized in Table 14. The level of potency attenuation by P-0885 and P-0882 in comparison to P-0880 followed the same trend across the cell subsets assessed, and were consistent with the level of reduction observed for P-0759 and P-0731 vs P-0704 (the respective Fc fusion proteins; FIGS. 15A & 15B), and P-0786 and P-0783 vs P-0782 (the respective mouse PD1 antibody fusion proteins; FIG. 19). Expectedly, the wild-type IL-2 fusion showed comparable activity on CD8+ T and NK cells as P-0880.

TABLE 14
Activity of IL-2 variant human PD-1 antibody fusion proteins

| | |
|---|---|
| pSTA5 EC₅₀ (pM) | Ki67 EC₅₀ (nM) |

| Compound | CD8+ T | NK | CD8+ T | NK |
|---|---|---|---|---|
| P-0880 | 73 | 643 | 1.7 | 3.6 |
| P-0885 | 249 | 2326 | 9.3 | 21 |
| P-0882 | 513 | 9920 | 77 | 230 |
| P-0849 | 65 | 934 | 3.3 | 4.1 |

### Example 14

### Pharmacodynamic Effect of IL-2 Variant Surrogate Mouse PD-1 Antibody Fusion Proteins in C57BL6 Mice

The pharmacodynamic effect of IL-2 variant mouse PD-1 antibody fusion proteins were assessed in C57BL6 mice following a single injection. Seven-week old female C57BL6 mice were received from Charles River Laboratory and acclimated in house for at least 7 days before the study. Vehicle, and a single dosing of each IL-2 mouse PD-1 antibody fusion protein was administered i.p. to mice at time 0. Blood samples were withdrawn on days 3, 5, 7, and 10 post injection. Each group included 5 mice. Heparin-treated whole blood was used for immune phenotyping described in Example 10.

P-0782 comprises an IL-2 P65R moiety that abrogated IL-2Rα binding, P-0838 comprises an IL-2 P65Q moiety that reduced IL-2Rα binding, while P-0837 contains a wild-type IL-2. P-0781, a counterpart mouse PD-1 antibody fusion protein containing a benchmark IL-2 variant (SEQ ID NO: 188) that completed lost binding to IL-2Rα, was included for comparison.

Following a single injection at 2 mg/kg, Ki67 stimulation achieved maximal levels for all compounds tested on CD8 and NK cells (FIGS. 27A-27B). The peak Ki67 expression signals for each compound reached maximum level on CD8+ T cells and peaked on day 3, For P-0782, P-0838 and Benchmark P-0781, the signal persisted through day 7 and diminished on day 10. In comparison, Ki67 signal weakened in an accelerated rate for wildtype P-0837 (FIG. 27A), Similar Ki67 induction on NK cells was observed for all compounds tested (FIG. 27B).

Strikingly, CD8 and NK cell expansion showed drastic difference among tested compounds. P-0782 with a mutation abolishing IL-2Rα binding showed vigorous expansion of CD8+ T (FIG. 27C) and NK cells (FIG. 27D). Expansion of both lymphocyte subsets started on day 3, continued and peaked on day 7 with a 68-fold increase in CD8+ T cells and 182-fold NK cell number increase. P-0838 containing the mutation with reduced IL-2Rα binding ability showed CD8 and NK cell expansion similar to or slightly stronger than WT antibody fusion. For the benchmark P-0781, expansion of both lymphocyte subsets was intermediate compared to P-0780 and P-0838. In sharp contrast, cell expansion of both lymphocyte subsets by wildtype P-0837 peaked on day 5 with significantly lower maximal signal (3.9-fold increases for CD8+ T cells and 6.8-fold for NK cells; FIGS. 27C and 27D)

It is possible that the CD25-binding abolishing mutation may provide advantage to reduce CD25 sink effect and consequently increase the availability to IL-2Rβγ. The enriched receptor occupancy elicits vigorous cytotoxic cell expansion. Mutants with residual CD25 binding activity may still have sink effect resulting in similar activity as wild type on CD8 and NK cells. In summary, P-0782 showed sharply different cell expansion profiles compared to P-0838 and P-0837. P-0782 demonstrated remarkable proliferation and expansion of both CD8+ T and NK cells compared to any compounds tested and is superior to the benchmark compound, P-0781. As an IL-2Rβγ-selective full agonist, P-0782 and P-0781 can bolster dramatical in vivo responses of CD8+ effector T and NK cells due to enhanced selectivity and reduced CD25 sink. Although P-0838 did not show strong CD8 and NK cell expansion compared to wild-type, the mutation introduced to reduce binding ability to IL-2Rα (CD25) is expected to provide benefits in reducing VLS. In addition, the residue immune regulatory Treg response may provide immune counterbalance to improve systemic tolerability and ensure the immune balance not tilted excessively to cytotoxic effector cells. The Treg response can be fine-tuned not to suffer tumor killing efficacy but strong enough to maintain peripheral tolerance.

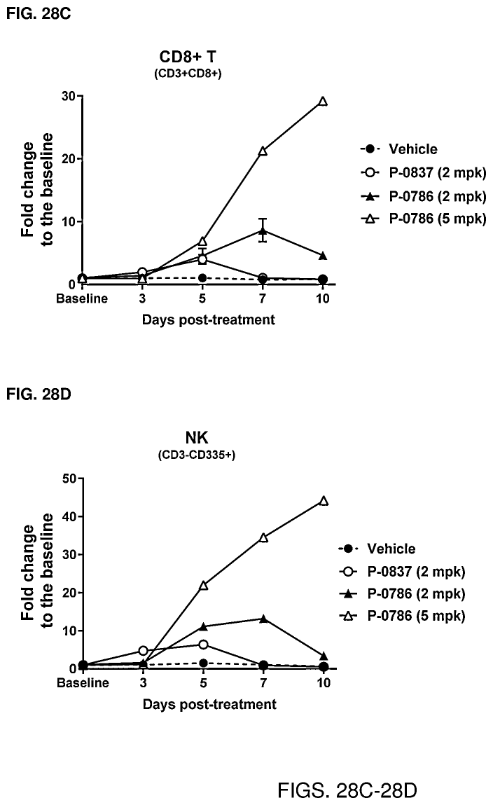

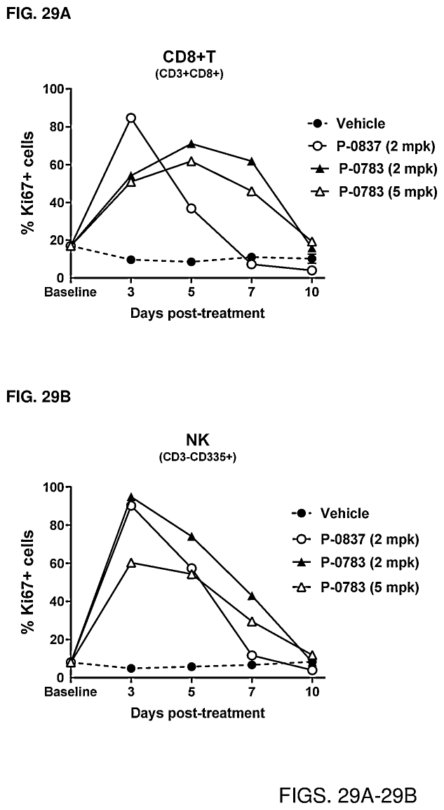

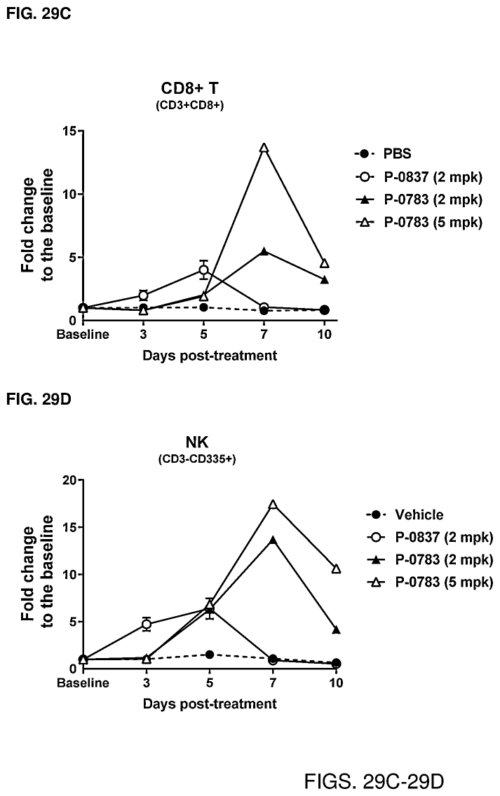

The pharmacodynamics of bifunctional PD1 antibody fusion proteins with IL-2 variants containing mutations to reduce IL-2Rβγ interaction in addition to abolishing binding capability to IL-2Rα was also tested. Both P-0786 and P-0783 are IL-2 potency-attenuated counterparts of P-0782 by incorporating different IL-2Rβ-modulating mutations L19Q and L19H, respectively. FIGS. 19C and 19D displayed the in vitro potency differences between these three compounds in stimulating Ki67 expression. The effect of P-0786 and P-0783 on proliferation and expansion of CD8+ and NK cell at two different dose levels are shown in FIGS. 28 and 29. As shown in FIG. 28A, the lower-potency compound, P-0786, induced peak Ki67 signal on CD8+ T cells on day 5 instead of day 3 observed for the wild-type P-0837. The increases in Ki67 on NK cells were maximized by both P-0783 and P-0837 (FIG. 28B), consistent with the notion that NK cells are more responsive to IL-2 than CD8+ T cell.

The pharmacodynamic effect of the PD1 antibody fusion of attenuated IL-2 variants was dramatically improved compared to wildtype fusion. FIGS. 28C and 28D demonstrated a remarkably prolonged and enhanced dose-response effect on cell expansion by P-0786 compared to wildtype. Increases in CD8+ T and NK cell expansion were delayed but persistent and durable. The response from 2 mg/kg dose group peaked on day 7 and did not return to baseline on Day 10, while the response from 5 mg/kg dose group sharply and continuously increased without reaching to the peak at Day 10 post dose. On the contrary, the CD8 and NK cell expansion in wildtype fusion group was marginal, peaked at day 5 and returned to baseline on day 7 (FIGS. 28C and 28D).

Comprising an even weaker IL-2 agonist, P-0783 showed similar delayed but persistent and durable effect as P-0786 in inducing Ki67 expression (FIGS. 29A and 29B) and expansion of CD8+ and NK cells in a dose-dependent manner (FIGS. 29C and 29D). The day on which the cell number peaked and fold change of cell number increases for each compound are summarized in Table 15.

TABLE 15
Peak peripheral cell numbers and fold change
over baseline following treatment

| | | CD8+ T cells | | NK cells | |
|---|---|---|---|---|---|
| | | Day the | | Day the | |
| | Dose level | signal | Fold | signal | Fold |
| Compound | (mg/kg) | peaked | ↑ | peaked | ↑ |
| P-0782 | 2 | 7 | 68 | 7 | 183 |
| P-0786 | 2 | 7 | 8.6 | 7 | 13 |
| | 5 | >10 | 29 | >10 | 44 |
| P-0783 | 2 | 7 | 5.5 | 7 | 14 |
| | 5 | 7 | 14 | 7 | 17 |
| P-0837 | 2 | 5 | 3.9 | 5 | 6.3 |

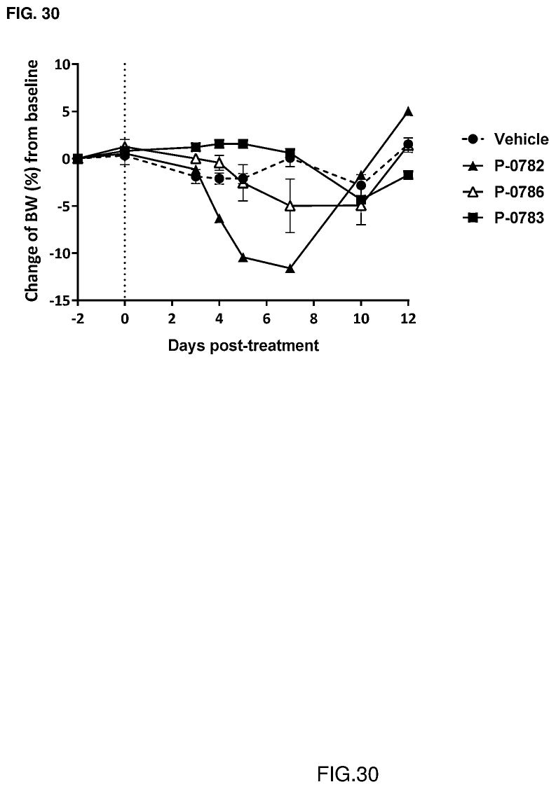

Further, as demonstrated in FIG. 30, the potency level and corresponding cytotoxic lymphocytes expansion correlated with the toxicity reflected by mice body weight losses. As an IL-2Rβγ-selective full agonist, P-0782 caused dramatic increases in both CD8+ T and NK cell numbers and resulted in the biggest weight loss; attenuated agonists P-0786 and P-0783 showed improved tolerability in vivo. P-0783 had a slight edge in tolerability than P-0786, consistent with the fact that P-0783 is a weaker agonist than P-0786.

In summary, P-0782 demonstrated a potent pharmacodynamic effect in proliferating and expanding CD8+ T and NK cells. P-0786 and P-0783 displayed weaker but more persistent signals. The potency ranking of the three compounds were in general agreement between ex vivo and in vivo. Further, potency attenuated compounds P-0786 and P-0783 showed improved pharmacodynamics and tolerability in vivo compared to the full agonist P-0782.

### Example 15

### In Vivo Efficacy of PD1 Antibody IL-2 Variant Fusion Proteins in Syngeneic Mouse Tumor Models

The anti-tumor efficacies of IL-2 variant mouse PD-1 antibody fusion proteins were tested in a subcutaneous B16F10 melanoma mouse tumor model. Female C57BL/6 mice (7 weeks) were randomized into treatment groups (n=10/group) by body weight after 4-7 days acclimation. B16F10 cells at passage 3 (5×10⁵ cells/mouse) were subcutaneously (s.c.) inoculated into the right flank of mice on day −1. Mice were administered intraperitoneally (i.p.) with tested compounds three times on days 0, 7 and 14 (Q7D). All the mice were closely monitored, and body weights were measured three times per week. Tumors were measured three time per week using the standard calipers, tumor size were calculated by standard formula Length×(width) w²×0.5 in mm³. Mice were euthanized as tumor size exceed the limit 1500 mm³.

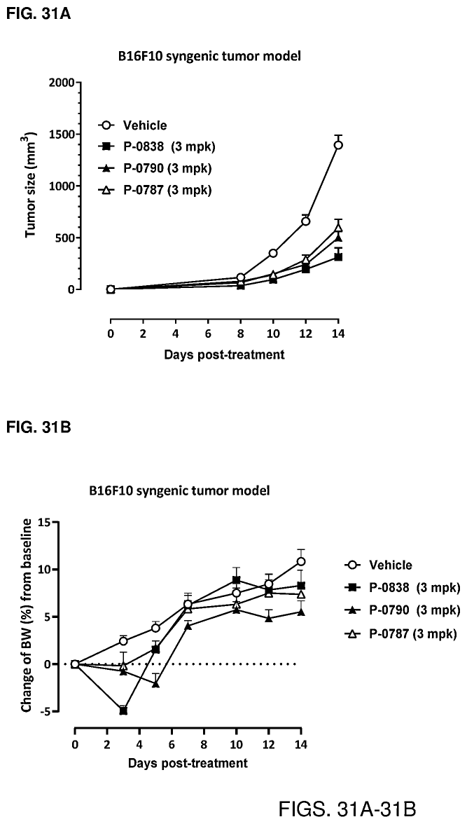

Three antibody fusion proteins, P-0838, P-0790, and P-0787, were dosed at 3 mg/kg with two Q7D doses. All three fusion proteins contain IL-2 L65Q mutation to impair binding to IL-2Rα; P-0790 and P-0787 comprise additional L19Q and L19H mutations, respectively, to further reduce IL-2Rβγ activity to modulate overall potency. As demonstrated in FIG. 31A, all compounds showed strong single-agent antitumor efficacy with 78%, 64%, and 57% of tumor growth inhibition for P-0787, P-0790, and P-0838, respectively. The level of tumor inhibitory efficacy correlated with the in vitro potency attenuation from P-0838 to P-0790 and to P-0787.

Similar to what was observed in FIG. 30, FIG. 31B depicted that full IL-2 agonist, P-0838, had the earliest and highest toxicity as reflected by the biggest weight loss; and attenuated agonists, P-0790 and P-0787, showed improved tolerability in vivo. P-0787 had some edge in tolerability than P-0790, consistent with the fact that P-0783 is a weaker agonist than P-0786. Overall, data support that IL-2Rβγ selective and attenuated mutants demonstrate good tumor killing efficacy and improved tolerability.

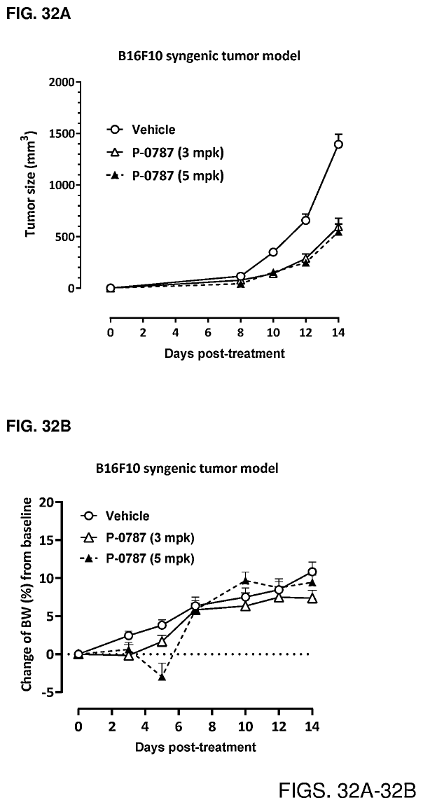

Dose effect on the tumor inhibition and tolerability in vivo were further investigated for P-0787. As seen in FIG. 32A, P-0787 showed similarly potent anti-tumor effect as dose increased from 3 mg/kg to 5 mg/kg. The dose escalation did not produce dramatic weight loss (FIG. 32B), suggesting a weak agonist facilitated high dose and increased tolerability.

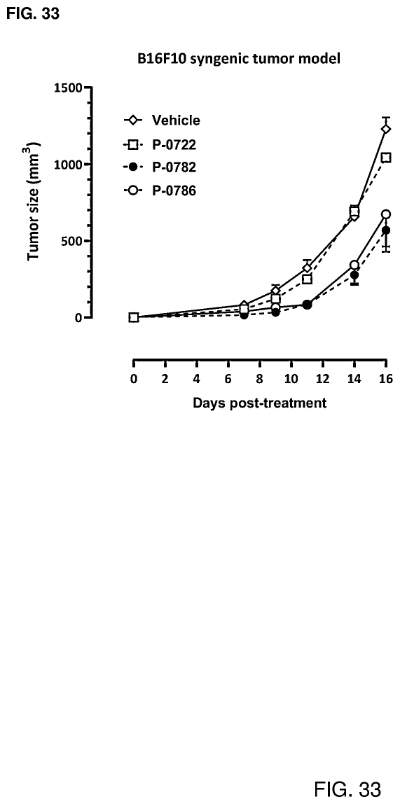

Strong tumor growth inhibition was also observed for P-0782 and P-0786 dosed at 1.5 mg/kg with 2 Q7D doses (FIG. 33). P-0722, the surrogate mouse PD-1 antibody, did not show anti-tumor effect in the B16F10 syngeneic model, whereas P-0782 and P-0786 showed comparable strong inhibition of tumor growth, despite that P-0786 is an attenuated counterpart of P-0782.

Finally, The IL-2 variant antibody fusion protein, P-0790, was tested in a mouse B16F10 pulmonary metastasis model. Briefly, 3×10⁵ mouse melanoma cells were intravenously injected into female B57BL6 mice (10-12 weeks old). Three Q7D treatments were initiated on the next day (day 1) via intraperitoneal injection. Treatment groups (n=5/group) includes P-0790 at 0.3, 1, and 3 mg/kg and corresponding antibody P-0722 at 3 mg/kg. Vehicle (PBS) was included as the negative control. On day 24, all mice were sacrificed for tissue harvesting. Lung tumor nodules were counted, and anti-metastatic effect were represented by different numbers of tumor nodules between treatment groups and vehicle control.

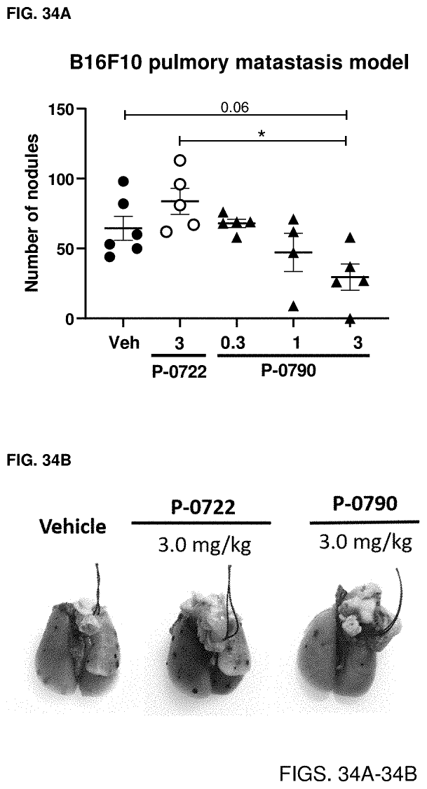

P-0790 is an IL-2 L19Q/P65Q PD-1 antibody fusion protein with significantly impaired binding to IL-2Rα and modulated overall potency. Similarly, P-0722, the surrogate mouse PD-1 antibody, was ineffective in inhibiting the metastasis of B16F10 tumor cells, whereas a dose-dependent inhibition of lung metastatic nodules by P-0790 was observed. FIG. 34A showed average lung nodule counts and FIG. 34B displayed lung picture of a representative animal from each group. Data are expressed as mean±SEM.

In summary, various IL-2 variant mouse PD-1 antibody fusion proteins demonstrated strong single-agent anti-tumor effects. Attenuated IL-2 agonists showed effective tumor growth inhibition and improved tolerability, which allowed for higher dose for improved efficacy.

### Example 16

### Pharmacodynamic/Pharmacokinetic and Safety Evaluation of Selected IL-2 Variant PD-1 Antibody Fusion Proteins in Cynomolgus Monkey

PK/PD properties and safety of selected IL-2 variant PD-1 antibody fusion proteins in cynomolgus monkey will be evaluated. Drug-naïve cynomolgus monkeys will be acclimated and trained for 2-3 weeks and randomized to one monkey per group, which will be followed by a pre-dose baseline week. On Day +1, one group will receive intravenous administration of vehicle (PBS), and other groups will be dosed intravenously with different test compounds.

Blood is collected on Day −3, 2, 4, 6, 8, 10, 12, 15. Peripheral blood mononuclear cells (PBMC) are isolated from monkey whole blood and used for FACS immunophenotyping of peripheral blood Treg, non-regulatory CD4+ T cells, CD8+ T cells, CD8+ T central memory, CD8+ effector memory, CD8+ T naïve and NK cells, to determine pharmacodynamics. Cell activation and proliferation will also be monitored by measuring CD25 and Ki67. Whole blood is also used for complete blood count (CBC) with 5-part differential: neutrophil, lymphocytes, monocytes, eosinophil, and basophil.

PK properties of selected IL-2 variant PD-1 antibody fusion proteins will be assessed in the cynomolgus plasma samples by measuring full-length intact molecule using mouse anti-human IL-2 Ab (BD Pharmingen) to coat 96-well plates in order to capture the fusion proteins. Mouse anti-human IL-2-biotin (in house) will be used for detection and the plasma concentrations of the test compounds will be subsequently quantified. In addition to the plasma samples collected on Day −3, 2, 3, 4, 5, 6, 8, 10, 15 and four more plasma samples were collected on day 1 at 10 minutes, 1 hour, 4 hours, and 8 hours post the administration of the selected IL-2 variant PD-1 antibody fusion proteins.

Plasma samples from days −7, 8, 15 will also be used to evaluate the following clinical chemistry parameters: aspartate aminotransferase, alanine aminotransferase, alkaline phosphatase, gamma glutamyl transferase, albumin, total bilirubin, creatinine, blood urea nitrogen, and C-reactive protein.

Further, body weight of each animal will be monitored weekly during the whole study period. Body temperature and blood pressure will be monitored on Day-1 (pre-dose) and 6 hours, 24 hours, 96 hours and 168 hours post the drug administration.

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

### SEQUENCE LISTINGS

The nucleic and amino acid sequences listed in the accompanying sequence listing are shown using standard letter abbreviations for nucleotide bases and one letter codes for amino acids, as defined in 37 C.F.R. 1.822.

SEQ ID NO: 1 is a human IL-2 precursor amino acid sequence.

SEQ ID NO: 2 is a human IL-2 mature form naturally occurring amino acid sequence.

SEQ ID NO: 3 is a human IL-2 mature form wild type amino acid sequence.

SEQ ID NO: 4 is a human IL-2 mature form amino acid sequence comprising 51251 substitution for improving fusion protein developability profiles.

SEQ ID NO: 5 is a human IL-2Rα extracellular domain amino acid sequence.

SEQ ID NO: 6 is a human IgG1-Fc amino acid sequence.

SEQ ID NO: 7 is a human IgG1-Fc sequence with reduced/abolished effector function.

SEQ ID NO: 8 is a human IgG1-Fc sequence with reduced/abolished effector function and extended half-life.

SEQ ID NO: 9 is a Knob-Fc amino acid sequence with reduced/abolished effector function.

SEQ ID NO: 10 is a Hole-Fc amino acid sequence with reduced/abolished effector function.

SEQ ID NOS: 11-30 are the amino acid sequences of various peptide linker sequences.

SEQ ID NOS: 31-66 are the amino acid sequences of various IL-2 variants with amino acid substitutions introduced at the interface with the IL-2 receptor a subunit.

SEQ ID NOS: 67-107 are the amino acid sequences of various IL-2 variant Fc fusion proteins.

SEQ ID NO: 108 is the amino acid sequence of Benchmark IL-2 variant Fc fusion protein.

SEQ ID NO: 109 is a human IL-2Rβ extracellular domain amino acid sequence.

SEQ ID NO: 110 is a human γc extracellular domain amino acid sequence.

SEQ ID NOS: 111-120 are the amino acid sequences of various IL-2 variants.

SEQ ID NOS: 121-133 are the amino acid sequences of various IL-2 variant Fc fusion proteins.

SEQ ID NO: 134 is a Knob-Fc amino acid sequence with reduced/abolished effector function and extended half-life.

SEQ ID NO: 135 is a Hole-Fc amino acid sequence with reduced/abolished effector function and extended half-life.

SEQ ID NOS: 136-137 are the amino acid sequences of the heavy chain and light chain of a humanized anti-FAP antibody.

SEQ ID NOS: 138-139 are the amino acid sequences of the heavy chain and light chain of a human PD-1 antagonist antibody.

SEQ ID NOS: 140-141 are the amino acid sequences of the heavy chain and light chain of a PD-1 antagonist antibody.

SEQ ID NOS: 142-143 are the amino acid sequences of the heavy chain and light chain of a PD-1 antagonist antibody.

SEQ ID NOS: 144-145 are the amino acid sequences of the heavy chain and light chain of a PD-1 antagonist antibody.

SEQ ID NOS: 146-147 are the amino acid sequences of the heavy chain and light chain of a PD-1 antagonist antibody.

SEQ ID NOS: 148-149 are the amino acid sequences of the heavy chain and light chain of a PD-L1 antagonist antibody.

SEQ ID NOS: 150-151 are the amino acid sequences of the heavy chain and light chain of a CTLA-4 antagonist antibody.

SEQ ID NOS: 152-153 are the amino acid sequences of the heavy chain and light chain of a CD40 agonist antibody.

SEQ ID NOS: 154-155 are the amino acid sequences of the heavy chain and light chain of a fibronectin antagonist antibody.

SEQ ID NOS: 156-157 are the amino acid sequences of the heavy chain and light chain of CD20 antagonist antibody.

SEQ ID NOS: 158-159 are the amino acid sequences of the heavy chain and light chain of a Her-2/neu antagonist antibody.

SEQ ID NOS: 160-161 are the amino acid sequences of the heavy chain and light chain of an EGFR antagonist antibody.

SEQ ID NO: 162 is the amino acid sequence of a human IgG1 CH1CH2CH3 domain sequence with reduced/abolished Fc effector function.

SEQ ID NO: 163 is the amino acid sequence of a human IgG1 CH1CH2CH3 domain knob chain sequence with reduced/abolished Fc effector function.

SEQ ID NO: 164 is the amino acid sequence of a human IgG1 CH1CH2CH3 domain hole chain sequence with reduced/abolished Fc effector function.

SEQ ID NOS: 165-169 are the amino acid sequences of various -IL-2 variant antibody fusion proteins.

SEQ ID NO: 170 is a human IL-2 receptor alpha Sushi domain amino acid sequence.

SEQ ID NOS: 171-174 are amino acid sequences of IL-2 and IL-2RSushi Fc fusion proteins.

SEQ ID NOS: 175-181 are amino acid sequences of the knob chains of various IL-2 variant human PD-1 antagonist antibody fusion proteins.

SEQ ID NOS: 182-184 are amino acid sequences of Benchmark IL-2 variant antibody fusion protein.

SEQ ID NOS: 185-187 are amino acid sequences of a surrogate anti-mouse PD-1 antibody with heterodimeric heavy chains.

SEQ ID NO: 188 is amino acid sequence of a Benchmark IL-2 variant.

SEQ ID NOS: 189-191 are amino acid sequences of the knob chains of various IL-2 variant human PD-1 antagonist antibody fusion proteins.

SEQUENCE LISTINGS
Human IL-2 precursor sequence
(SEQ ID NO: 1)
MYRMQLLSCIALSLALVINSAPTSSSIKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT
Human IL-2 mature form naturally occurring
sequence
(SEQ ID NO: 2)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
Human IL-2 mature form wild-type sequence
(SEQ ID NO: 3)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFSQSIISTLT
Human IL-2 S125I variant sequence
(SEQ ID NO: 4)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFIQSIISTLT
Human IL-2Rα (CD25) extracellular
domain sequence
(SEQ ID NO: 5)
ELCDDDPPEIPHATFKAMAYKEGTMLNCECKRGFRRIKSGSLYML
CTGNSSHSSWDNQCQCTSSATRNTTKQVTPQPEEQKERKTTEMQS
PMQPVDQASLPGHCREPPPWENEATERIYHFVVGQMVYYQCVQGY
RALHRGPAESVCKMTHGKTRWTQPQLICTGEMETSQFPGEEKPQA
SPEGRPESETSCLVITTDFQIQTEMAATMETSIFTTEYQ
Human IgG1-Fc
(SEQ ID NO: 6)
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G
Human IgG1-Fc with reduced/abolished
effector function
(SEQ ID NO: 7)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G
Human IgG1-Fc with reduced/abolished effector
function and extended half-life
(SEQ ID NO: 8)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHAHYTQKSLSLSP
G
Human IgG Knob-Fc with reduced/abolished
effector function
(SEQ ID NO: 9)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
G
Human IgG Hole-Fc with reduced/abolished
effector function
(SEQ ID NO: 10)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
EEMTKNQVSLSCAVKGFYPSDIAVEWESNGQPENNYKTTPPVLDS
DGSFFLVSKLTVDKSRWQQGNVFSCSVMHEALHNHYTQKSLSLSP
G
Peptide linker sequence
(SEQ ID NO: 11)
GGGSGGGSGGGS
Peptide linker sequence
(SEQ ID NO: 12)
GGGS
Peptide linker sequence
(SEQ ID NO: 13)
GSSGGSGGSGGSG
Peptide linker sequence
(SEQ ID NO: 14)
GSSGT
Peptide linker sequence
(SEQ ID NO: 15)
GGGGSGGGGSGGGGS
Peptide linker sequence
(SEQ ID NO: 16)
AEAAAKEAAAKEAAAKA
Peptide linker sequence
(SEQ ID NO: 17)
GGGGSGGGGSGGGGSGGGGS
Peptide linker sequence
(SEQ ID NO: 18)
GGGSGGGS
Peptide linker sequence
(SEQ ID NO: 19)
GSGST
Peptide linker sequence
(SEQ ID NO: 20)
GGSS
Peptide linker sequence
(SEQ ID NO: 21)
GGGGS
Peptide linker sequence
(SEQ ID NO: 22)
GGSG
Peptide linker sequence
(SEQ ID NO: 23)
SGGG
Peptide linker sequence
(SEQ ID NO: 24)
GSGS
Peptide linker sequence
(SEQ ID NO: 25)
GSGSGS
Peptide linker sequence
(SEQ ID NO: 26)
GSGSGSGS
Peptide linker sequence
(SEQ ID NO: 27)
GSGSGSGSGS
Peptide linker sequence
(SEQ ID NO: 28)
GSGSGSGSGSGS
Peptide linker sequence
(SEQ ID NO: 29)
GGGGSGGGGS
Peptide linker sequence
(SEQ ID NO: 30)
GSGSGSGSGSGSGGS
IL-2 F42A/S125I variant sequence
(SEQ ID NO: 31)
APTSSSTKKTQLQLEHLLLDLQMILNGINNYKNPKLTRMLTAKFY
MPKKATELKHLQCLEEELKPLEEVLNLAQSKNFHLRPRDLISNIN
VIVLELKGSETTFMCEYADETATIVEFLNRWITFIQSIISTLT
IL-2 R38F/S125I variant sequence
(SEQ ID NO: 32)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFIQSIISTLT
IL-2 R38G/S125I variant sequence
(SEQ ID NO: 33)
APTSSSTKKTQLQLEHLLLDLQMILNGINNYKNPKLTGMLTAKFY
MPKKATELKHLQCLEEELKPLEEVLNLAQSKNFHLRPRDLISNIN
VIVLELKGSETTFMCEYADETATIVEFLNRWITFIQSIISTLT
IL-2 R38A/S125I variant sequence
(SEQ ID NO: 34)
APTSSSTKKTQLQLEHLLLDLQMILNGINNYKNPKLTAMLTFKFY
MPKKATELKHLQCLEEELKPLEEVLNLAQSKNFHLRPRDLISNIN
VIVLELKGSETTFMCEYADETATIVEFLNRWITFIQSIISTLT
IL-2 T41A/S125I variant sequence
(SEQ ID NO: 35)
APTSSSTKKTQLQLEHLLLDLQMILNGINNYKNPKLTRMLAFKFY
MPKKATELKHLQCLEEELKPLEEVLNLAQSKNFHLRPRDLISNIN
VIVLELKGSETTFMCEYADETATIVEFLNRWITFIQSIISTLT
IL-2 T41G/S125I variant sequence
(SEQ ID NO: 36)
APTSSSTKKTQLQLEHLLLDLQMILNGINNYKNPKLTRMLGFKFY
MPKKATELKHLQCLEEELKPLEEVLNLAQSKNFHLRPRDLISNIN
VIVLELKGSETTFMCEYADETATIVEFLNRWITFIQSIISTLT
IL-2 T41V/S125I variant sequence
(SEQ ID NO: 37)
APTSSSTKKTQLQLEHLLLDLQMILNGINNYKNPKLTRMLVFKFY
MPKKATELKHLQCLEEELKPLEEVLNLAQSKNFHLRPRDLISNIN
VIVLELKGSETTFMCEYADETATIVEFLNRWITFIQSIISTLT
IL-2 F44G/S125I variant sequence
(SEQ ID NO: 38)
APTSSSTKKTQLQLEHLLLDLQMILNGINNYKNPKLTRMLTFKGY
MPKKATELKHLQCLEEELKPLEEVLNLAQSKNFHLRPRDLISNIN
VIVLELKGSETTFMCEYADETATIVEFLNRWITFIQSIISTLT
IL-2 F44V/S125I variant sequence
(SEQ ID NO: 39)
APTSSSTKKTQLQLEHLLLDLQMILNGINNYKNPKLTRMLTFKVY
MPKKATELKHLQCLEEELKPLEEVLNLAQSKNFHLRPRDLISNIN
VIVLELKGSETTFMCEYADETATIVEFLNRWITFIQSIISTLT
IL-2 E62A/S125I variant sequence
(SEQ ID NO: 40)
APTSSSTKKTQLQLEHLLLDLQMILNGINNYKNPKLTRMLTFKFY
MPKKATELKHLQCLEEALKPLEEVLNLAQSKNFHLRPRDLISNIN
VIVLELKGSETTFMCEYADETATIVEFLNRWITFIQSIISTLT
IL-2 E62F/S125I variant sequence
(SEQ ID NO: 41)
APTSSSTKKTQLQLEHLLLDLQMILNGINNYKNPKLTRMLTFKFY
MPKKATELKHLQCLEEFLKPLEEVLNLAQSKNFHLRPRDLISNIN
VIVLELKGSETTFMCEYADETATIVEFLNRWITFIQSIISTLT
IL-2 E62H/S125I variant sequence
(SEQ ID NO: 42)
APTSSSTKKTQLQLEHLLLDLQMILNGINNYKNPKLTRMLTFKFY
MPKKATELKHLQCLEEHLKPLEEVLNLAQSKNFHLRPRDLISNIN
VIVLELKGSETTFMCEYADETATIVEFLNRWITFIQSIISTLT
IL-2 E62L/S125I variant sequence
(SEQ ID NO: 43)
APTSSSTKKTQLQLEHLLLDLQMILNGINNYKNPKLTRMLTFKFY
MPKKATELKHLQCLEELLKPLEEVLNLAQSKNFHLRPRDLISNIN
VIVLELKGSETTFMCEYADETATIVEFLNRWITFIQSIISTLT
IL-2 P65G/S125I variant sequence
(SEQ ID NO: 44)
APTSSSTKKTQLQLEHLLLDLQMILNGINNYKNPKLTRMLTFKFY
MPKKATELKHLQCLEEELKGLEEVLNLAQSKNFHLRPRDLISNIN
VIVLELKGSETTFMCEYADETATIVEFLNRWITFIQSIISTLT
IL-2 P65E/S125I variant sequence
(SEQ ID NO: 45)
APTSSSTKKTQLQLEHLLLDLQMILNGINNYKNPKLTRMLTFKFY
MPKKATELKHLQCLEEELKELEEVLNLAQSKNFHLRPRDLISNIN
VIVLELKGSETTFMCEYADETATIVEFLNRWITFIQSIISTLT
IL-2 P65H/S125I variant sequence
(SEQ ID NO: 46)
APTSSSTKKTQLQLEHLLLDLQMILNGINNYKNPKLTRMLTFKF
YMPKKATELKHLQCLEEELKHLEEVLNLAQSKNFHLRPRDLISN
INVIVLELKGSETTFMCEYADETATIVEFLNRWITFIQSIISTL
T
IL-2 P65R/S125I variant sequence
(SEQ ID NO: 47)
APTSSSTKKTQLQLEHLLLDLQMILNGINNYKNPKLTRMLTFKFY
MPKKATELKHLQCLEEELKRLEEVLNLAQSKNFHLRPRDLISNIN
VIVLELKGSETTFMCEYADETATIVEFLNRWITFIQSIISTLT
IL-2 P65A/S125I variant sequence
(SEQ ID NO: 48)
APTSSSTKKTQLQLEHLLLDLQMILNGINNYKNPKLTRMLTFKFY
MPKKATELKHLQCLEEELKALEEVLNLAQSKNFHLRPRDLISNIN
VIVLELKGSETTFMCEYADETATIVEFLNRWITFIQSIISTLT
IL-2 P65K/S125I variant sequence
(SEQ ID NO: 49)
APTSSSTKKTQLQLEHLLLDLQMILNGINNYKNPKLTRMLTFKFY
MPKKATELKHLQCLEEELKKLEEVLNLAQSKNFHLRPRDLISNIN
VIVLELKGSETTFMCEYADETATIVEFLNRWITFIQSIISTLT
IL-2 P65N/S125I variant sequence
(SEQ ID NO: 50)
APTSSSTKKTQLQLEHLLLDLQMILNGINNYKNPKLTRMLTFKFY
MPKKATELKHLQCLEEELKNLEEVLNLAQSKNFHLRPRDLISNIN
VIVLELKGSETTFMCEYADETATIVEFLNRWITFIQSIISTLT
IL-2 P650/S125I variant sequence
(SEQ ID NO: 51)
APTSSSTKKTQLQLEHLLLDLQMILNGINNYKNPKLTRMLTFKFY
MPKKATELKHLQCLEEELKQLEEVLNLAQSKNFHLRPRDLISNIN
VIVLELKGSETTFMCEYADETATIVEFLNRWITFIQSIISTLT
IL-2 E68A/S125I variant sequence
(SEQ ID NO: 52)
APTSSSTKKTQLQLEHLLLDLQMILNGINNYKNPKLTRMLTFKFY
MPKKATELKHLQCLEEELKPLEAVLNLAQSKNFHLRPRDLISNIN
VIVLELKGSETTFMCEYADETATIVEFLNRWITFIQSIISTLT
IL-2 E68F/S125I variant sequence
(SEQ ID NO: 53)
APTSSSTKKTQLQLEHLLLDLQMILNGINNYKNPKLTRMLTFKFY
MPKKATELKHLQCLEEELKPLEFVLNLAQSKNFHLRPRDLISNIN
VIVLELKGSETTFMCEYADETATIVEFLNRWITFIQSIISTLT
IL-2 E68H/S125I variant sequence
(SEQ ID NO: 54)
APTSSSTKKTQLQLEHLLLDLQMILNGINNYKNPKLTRMLTFKFY
MPKKATELKHLQCLEEELKPLEHVLNLAQSKNFHLRPRDLISNIN
VIVLELKGSETTFMCEYADETATIVEFLNRWITFIQSIISTLT
IL-2 E68L/S125I variant sequence
(SEQ ID NO: 55)
APTSSSTKKTQLQLEHLLLDLQMILNGINNYKNPKLTRMLTFKFY
MPKKATELKHLQCLEEELKPLELVLNLAQSKNFHLRPRDLISNIN
VIVLELKGSETTFMCEYADETATIVEFLNRWITFIQSIISTLT
IL-2 E68P/S125I variant sequence
(SEQ ID NO: 56)
APTSSSTKKTQLQLEHLLLDLQMILNGINNYKNPKLTRMLTFKFY
MPKKATELKHLQCLEEELKPLEPVLNLAQSKNFHLRPRDLISNIN
VIVLELKGSETTFMCEYADETATIVEFLNRWITFIQSIISTLT
IL-2 Y107G/S125I variant sequence
(SEQ ID NO: 57)
APTSSSTKKTQLQLEHLLLDLQMILNGINNYKNPKLTRMLTFKVY
MPKKATELKHLQCLEEELKPLEEVLNLAQSKNFHLRPRDLISNIN
VIVLELKGSETTFMCEGADETATIVEFLNRWITFIQSIISTLT
IL-2 Y107H/S125I variant sequence
(SEQ ID NO: 58)
APTSSSTKKTQLQLEHLLLDLQMILNGINNYKNPKLTRMLTFKVY
MPKKATELKHLQCLEEELKPLEEVLNLAQSKNFHLRPRDLISNIN
VIVLELKGSETTFMCEHADETATIVEFLNRWITFIQSIISTLT
IL-2 Y107L/S125I variant sequence
(SEQ ID NO: 59)
APTSSSTKKTQLQLEHLLLDLQMILNGINNYKNPKLTRMLTFKVY
MPKKATELKHLQCLEEELKPLEEVLNLAQSKNFHLRPRDLISNIN
VIVLELKGSETTFMCELADETATIVEFLNRWITFIQSIISTLT
IL-2 Y107V/S125I variant sequence
(SEQ ID NO: 60)
APTSSSTKKTQLQLEHLLLDLQMILNGINNYKNPKLTRMLTFKVY
MPKKATELKHLQCLEEELKPLEEVLNLAQSKNFHLRPRDLISNIN
VIVLELKGSETTFMCEVADETATIVEFLNRWITFIQSIISTLT
IL-2 F42A/E62F/S125I variant sequence
(SEQ ID NO: 61)
APTSSSTKKTQLQLEHLLLDLQMILNGINNYKNPKLTRMLTAKFY
MPKKATELKHLQCLEEFLKPLEEVLNLAQSKNFHLRPRDLISNIN
VIVLELKGSETTFMCEYADETATIVEFLNRWITFIQSIISTLT
IL-2 F42A/E62A/S125I variant sequence
(SEQ ID NO: 62)
APTSSSTKKTQLQLEHLLLDLQMILNGINNYKNPKLTRMLTAKFY
MPKKATELKHLQCLEEALKPLEEVLNLAQSKNFHLRPRDLISNIN
VIVLELKGSETTFMCEYADETATIVEFLNRWITFIQSIISTLT
IL-2 F42A/E62H/S125I variant sequence
(SEQ ID NO: 63)
APTSSSTKKTQLQLEHLLLDLQMILNGINNYKNPKLTRMLTAKFY
MPKKATELKHLQCLEEHLKPLEEVLNLAQSKNFHLRPRDLISNIN
VIVLELKGSETTFMCEYADETATIVEFLNRWITFIQSIISTLT
IL-2 F42A/P65H/S125I variant sequence
(SEQ ID NO: 64)
APTSSSTKKTQLQLEHLLLDLQMILNGINNYKNPKLTRMLTAKFY
MPKKATELKHLQCLEEELKHLEEVLNLAQSKNFHLRPRDLISNIN
VIVLELKGSETTFMCEYADETATIVEFLNRWITFIQSIISTLT
IL-2 F42A/P65R/S125I variant sequence
(SEQ ID NO: 65)
APTSSSTKKTQLQLEHLLLDLQMILNGINNYKNPKLTRMLTAKFY
MPKKATELKHLQCLEEELKRLEEVLNLAQSKNFHLRPRDLISNIN
VIVLELKGSETTFMCEYADETATIVEFLNRWITFIQSIISTLT
IL-2 F42A/P65A/S125I variant sequence
(SEQ ID NO: 66)
APTSSSTKKTQLQLEHLLLDLQMILNGINNYKNPKLTRMLTAKFY
MPKKATELKHLQCLEEELKALEEVLNLAQSKNFHLRPRDLISNIN
VIVLELKGSETTFMCEYADETATIVEFLNRWITFIQSIISTLT
P-0250
(SEQ ID NO: 67)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKPLEEVLNLAQSKNFHLR
PRDLISNINVIVLELKGSETTFMCEYADETATIVEFLNRWITFSQ
SIISTLT
P-0531
(SEQ ID NO: 68)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKPLEEVLNLAQSKNFHLR
PRDLISNINVIVLELKGSETTFMCEYADETATIVEFLNRWITFIQ
SIISTLT
P-0613
(SEQ ID NO: 69)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEELKPLEEVLNLAQSKNFHLR
PRDLISNINVIVLELKGSETTFMCEYADETATIVEFLNRWITFIQ
SIISTLT
P-0614
(SEQ ID NO: 70)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TFMLTFKFYMPKKATELKHLQCLEEELKPLEEVLNLAQSKNFHLR
PRDLISNINVIVLELKGSETTFMCEYADETATIVEFLNRWITFIQ
SIISTLT
P-0615
(SEQ ID NO: 71)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TGMLTFKFYMPKKATELKHLQCLEEELKPLEEVLNLAQSKNFHLR
PRDLISNINVIVLELKGSETTFMCEYADETATIVEFLNRWITFIQ
SIISTLT
P-0602
(SEQ ID NO: 72)
DKTHTCPPCPAPEAAGAPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVMHEALHNHYTQKSLSLSPG
GGGSGGGSAPTSSSIKKTQLQLEHLLLDLQMILNGINNYKNPKLT
AMLTFKFYMPKKATELKHLQCLEEELKPLEEVLNLAQSKNFHLRP
RDLISNINVIVLELKGSETTFMCEYADETATIVEFLNRWITFIQS
IISTLT
P-0603
(SEQ ID NO: 73)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLAFKFYMPKKATELKHLQCLEEELKPLEEVLNLAQSKNFHLR
PRDLISNINVIVLELKGSETTFMCEYADETATIVEFLNRWITFIQ
SIISTLT
P-0604
(SEQ ID NO: 74)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLGFKFYMPKKATELKHLQCLEEELKPLEEVLNLAQSKNFHLR
PRDLISNINVIVLELKGSETTFMCEYADETATIVEFLNRWITFIQ
SIISTLT
P-0605
(SEQ ID NO: 75)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLVFKFYMPKKATELKHLQCLEEELKPLEEVLNLAQSKNFHLR
PRDLISNINVIVLELKGSETTFMCEYADETATIVEFLNRWITFIQ
SIISTLT
P-0606
(SEQ ID NO: 76)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKGYMPKKATELKHLQCLEEELKPLEEVLNLAQSKNFHLR
PRDLISNINVIVLELKGSETTFMCEYADETATIVEFLNRWITFIQ
SIISTLT
P-0607
(SEQ ID NO: 77)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKVYMPKKATELKHLQCLEEELKPLEEVLNLAQSKNFHLR
PRDLISNINVIVLELKGSETTFMCEYADETATIVEFLNRWITFIQ
SIISTLT
P-0624
(SEQ ID NO: 78)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEALKPLEEVLNLAQSKNFHLR
PRDLISNINVIVLELKGSETTFMCEYADETATIVEFLNRWITFIQ
SIISTLT
P-0625
(SEQ ID NO: 79)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEFLKPLEEVLNLAQSKNFHLR
PRDLISNINVIVLELKGSETTFMCEYADETATIVEFLNRWITFIQ
SIISTLT
P-0626
(SEQ ID NO: 80)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEHLKPLEEVLNLAQSKNFHLR
PRDLISNINVIVLELKGSETTFMCEYADETATIVEFLNRWITFIQ
SIISTLT
P-0627
(SEQ ID NO: 81)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEELLKPLEEVLNLAQSKNFHLR
PRDLISNINVIVLELKGSETTFMCEYADETATIVEFLNRWITFIQ
SIISTLT
P-0608
(SEQ ID NO: 82)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKGLEEVLNLAQSKNFHLR
PRDLISNINVIVLELKGSETTFMCEYADETATIVEFLNRWITFIQ
SIISTLT
P-0633
(SEQ ID NO: 83)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKELEEVLNLAQSKNFHLR
PRDLISNINVIVLELKGSETTFMCEYADETATIVEFLNRWITFIQ
SIISTLT
P-0634
(SEQ ID NO: 84)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKHLEEVLNLAQSKNFHLR
PRDLISNINVIVLELKGSETTFMCEYADETATIVEFLNRWITFIQ
SIISTLT
P-0635
(SEQ ID NO: 85)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0628
(SEQ ID NO: 86)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKPLEAVLNLAQSKNFHLR
PRDLISNINVIVLELKGSETTFMCEYADETATIVEFLNRWITFIQ
SIISTLT
P-0629
(SEQ ID NO: 87)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKPLEFVLNLAQSKNFHLR
PRDLISNINVIVLELKGSETTFMCEYADETATIVEFLNRWITFIQ
SIISTLT
P-0630
(SEQ ID NO: 88)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKPLEHVLNLAQSKNFHLR
PRDLISNINVIVLELKGSETTFMCEYADETATIVEFLNRWITFIQ
SIISTLT
P-0631
(SEQ ID NO: 89)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKPLELVLNLAQSKNFHLR
PRDLISNINVIVLELKGSETTFMCEYADETATIVEFLNRWITFIQ
SIISTLT
P-0632
(SEQ ID NO: 90)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKFYMPKKATELKHLQCLEEELKPLEPVLNLAQSKNFHLR
PRDLISNINVIVLELKGSETTFMCEYADETATIVEFLNRWITFIQ
SIISTLT
P-0609
(SEQ ID NO: 91)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKVYMPKKATELKHLQCLEEELKPLEEVLNLAQSKNFHLR
PRDLISNINVIVLELKGSETTFMCEGADETATIVEFLNRWITFIQ
SIISTLT
P-0610
(SEQ ID NO: 92)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSIKKTQLQLEHLLLDLQMILNGINNYKNPKL
TRMLTFKVYMPKKATELKHLQCLEEELKPLEEVLNLAQSKNFHLR
PRDLISNINVIVLELKGSETTFMCEHADETATIVEFLNRWITFIQ
SIISTLT
P-0611
(SEQ ID NO: 93)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKVYMPKKATELKHLQCLEEELKPLEEVLNLAQSKNFHLR
PRDLISNINVIVLELKGSETTFMCELADETATIVEFLNRWITFIQ
SIISTLT
P-0612
(SEQ ID NO: 94)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKVYMPKKATELKHLQCLEEELKPLEEVLNLAQSKNFHLR
PRDLISNINVIVLELKGSETTFMCEVADETATIVEFLNRWITFIQ
SIISTLT
P-0551
(SEQ ID NO: 95)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFAMPKKATELKHLQCLEEELKPLEEVLNGAQSKNFHLR
PRDLISNINVIVLELKGSETTFMCEYADETATIVEFLNRWITFIQ
SIISTLT
P-0704 knob chain
(SEQ ID NO: 96)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0706 knob chain
(SEQ ID NO: 97)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKALEEVLNLAQSKNFHLR
PRDLISNINVIVLELKGSETTFMCEYADETATIVEFLNRWITFIQ
SIISTLT
P-0707 knob chain
(SEQ ID NO: 98)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKKLEEVLNLAQSKNFHLR
PRDLISNINVIVLELKGSETTFMCEYADETATIVEFLNRWITFIQ
SIISTLT
P-0708 knob chain
(SEQ ID NO: 99)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKNLEEVLNLAQSKNFHLR
PRDLISNINVIVLELKGSETTFMCEYADETATIVEFLNRWITFIQ
SIISTLT
P-0709 knob chain
(SEQ ID NO: 100)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKQLEEVLNLAQSKNFHLR
PRDLISNINVIVLELKGSETTFMCEYADETATIVEFLNRWITFIQ
SIISTLT
P-0702 knob chain
(SEQ ID NO: 101)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEFLKPLEEVLNLAQSKNFHLR
PRDLISNINVIVLELKGSETTFMCEYADETATIVEFLNRWITFIQ
SIISTLT
P-0766 knob chain
(SEQ ID NO: 102)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEALKPLEEVLNLAQSKNFHLR
PRDLISNINVIVLELKGSETTFMCEYADETATIVEFLNRWITFIQ
SIISTLT
P-0767 knob chain
(SEQ ID NO: 103)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEHLKPLEEVLNLAQSKNFHLR
PRDLISNINVIVLELKGSETTFMCEYADETATIVEFLNRWITFIQ
SIISTLT
P-0703 knob chain
(SEQ ID NO: 104)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEELKHLEEVLNLAQSKNFHLR
PRDLISNINVIVLELKGSETTFMCEYADETATIVEFLNRWITFIQ
SIISTLT
P-0705 knob chain
(SEQ ID NO: 105)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEELKRLEEVLNLAQSKNFHLR
PRDLISNINVIVLELKGSETTFMCEYADETATIVEFLNRWITFIQ
SIISTLT
P-0765 knob chain
(SEQ ID NO: 106)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFYMPKKATELKHLQCLEEELKALEEVLNLAQSKNFHLR
PRDLISNINVIVLELKGSETTFMCEYADETATIVEFLNRWITFIQ
SIISTLT
P-0689 knob chain
(SEQ ID NO: 107)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKPLEEVLNLAQSKNFHLR
PRDLISNINVIVLELKGSETTFMCEYADETATIVEFLNRWITFIQ
SIISTLT
Benchmark knob chain
(SEQ ID NO: 108)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTAKFAMPKKATELKHLQCLEEELKPLEEVLNGAQSKNFHLR
PRDLISNINVIVLELKGSETTFMCEYADETATIVEFLNRWITFIQ
SIISTLT
Human
IL-2Rβ (CD122) extracellular domain sequence
(SEQ ID NO: 109)
AVNGTSQFTCFYNSRANISCVWSQDGALQDTSCQVHAWPDRRRWN
QTCELLPVSQASWACNLILGAPDSQKLTTVDIVTLRVLCREGVRW
RVMAIQDFKPFENLRLMAPISLQVVHVETHRCNISWEISQASHYF
ERHLEFEARTLSPGHTWEEAPLLTLKQKQEWICLETLTPDTQYEF
QVRVKPLQGEFTTWSPWSQPLAFRTKPAALGKDT
Human common subunit gamma γ_{c} (CD132)
extracellular odmain sequence
(SEQ ID NO: 110)
LNTTILTPNGNEDTTADFFLTTMPTDSLSVSTLPLPEVQCFVFNV
EYMNCTWNSSSEPQPTNLTLHYWYKNSDNDKVQKCSHYLFSEEIT
SGCQLQKKEIHLYQTFVVQLQDPREPRRQATQMLKLQNLVIPWAP
ENLTLHKLSESQLELNWNNRFLNHCLEHLVQYRTDWDHSWTEQSV
DYRHKFSLPSVDGQKRYTFRVRSRFNPLCGSAQHWSEWSHPIHWG
SNTSKENPFLFALEA
IL-2 L19H/P65R/S125I variant sequence
(SEQ ID NO: 111)
APTSSSIKKTQLQLEHLLHDLQMILNGINNYKNPKLTRMLTFKFY
MPKKATELKHLQCLEEELKRLEEVLNLAQSKNFHLRPRDLISNIN
VIVLELKGSETTFMCEYADETATIVEFLNRWITFIQSIISTLT
IL-2 L19Q/P65R/S125I variant sequence
(SEQ ID NO: 112)
APTSSSTKKTQLQLEHLLQDLQMILNGINNYKNPKLTRMLTFKFY
MPKKATELKHLQCLEEELKRLEEVLNLAQSKNFHLRPRDLISNIN
VIVLELKGSETTFMCEYADETATIVEFLNRWITFIQSIISTLT
IL-2 L19Y/P65R/S125I variant sequence
(SEQ ID NO: 113)
APTSSSTKKTQLQLEHLLYDLQMILNGINNYKNPKLTRMLTFKFY
MPKKATELKHLQCLEEELKRLEEVLNLAQSKNFHLRPRDLISNIN
VIVLELKGSETTFMCEYADETATIVEFLNRWITFIQSIISTLT
IL-2 L19H/P65Q/S125I variant sequence
(SEQ ID NO: 114)
APTSSSIKKTQLQLEHLLHDLQMILNGINNYKNPKLTRMLTFKFY
MPKKATELKHLQCLEEELKQLEEVLNLAQSKNFHLRPRDLISNIN
VIVLELKGSETTFMCEYADETATIVEFLNRWITFIQSIISTLT
IL-2 L19H/P65H/S125I variant sequence
(SEQ ID NO: 115)
APTSSSIKKTQLQLEHLLHDLQMILNGINNYKNPKLTRMLTFKFY
MPKKATELKHLQCLEEELKHLEEVLNLAQSKNFHLRPRDLISNIN
VIVLELKGSETTFMCEYADETATIVEFLNRWITFIQSIISTLT
IL-2 L19H/P65N/S125I variant sequence
(SEQ ID NO: 116)
APTSSSIKKTQLQLEHLLHDLQMILNGINNYKNPKLTRMLTFKFY
MPKKATELKHLQCLEEELKNLEEVLNLAQSKNFHLRPRDLISNIN
VIVLELKGSETTFMCEYADETATIVEFLNRWITFIQSIISTLT
IL-2 L19Q/P65Q/S125I variant sequence
(SEQ ID NO: 117)
APTSSSTKKTQLQLEHLLQDLQMILNGINNYKNPKLTRMLTFKFY
MPKKATELKHLQCLEEELKQLEEVLNLAQSKNFHLRPRDLISNIN
VIVLELKGSETTFMCEYADETATIVEFLNRWITFIQSIISTLT
IL-2 L19Q/P65H/S125I variant sequence
(SEQ ID NO: 118)
APTSSSTKKTQLQLEHLLQDLQMILNGINNYKNPKLTRMLTFKFY
MPKKATELKHLQCLEEELKHLEEVLNLAQSKNFHLRPRDLISNIN
VIVLELKGSETTFMCEYADETATIVEFLNRWITFIQSIISTLT
IL-2 L19Q/P65N/S125I variant sequence
(SEQ ID NO: 119)
APTSSSTKKTQLQLEHLLQDLQMILNGINNYKNPKLTRMLTFKFY
MPKKATELKHLQCLEEELKNLEEVLNLAQSKNFHLRPRDLISNIN
VIVLELKGSETTFMCEYADETATIVEFLNRWITFIQSIISTLT
IL-2 P65R/S125I/0126E variant sequence
(SEQ ID NO: 120)
APTSSSTKKTQLQLEHLLLDLQMILNGINNYKNPKLTRMLTFKFY
MPKKATELKHLQCLEEELKRLEEVLNLAQSKNFHLRPRDLISNIN
VIVLELKGSETTFMCEYADETATIVEFLNRWITFIESIISTLT
P-0731 knob chain
(SEQ ID NO: 121)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLHDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0759 knob chain
(SEQ ID NO: 122)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLQDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0761 knob chain
(SEQ ID NO: 123)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLYDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0811 knob chain
(SEQ ID NO: 124)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLHDLQMILNGINNYKNPKL
TRMLTFKFYMPKKATELKHLQCLEEELKQLEEVLNLAQSKNFHLR
PRDLISNINVIVLELKGSETTFMCEYADETATIVEFLNRWITFIQ
SIISTLT
P-0812 knob chain
(SEQ ID NO: 125)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLHDLQMILNGINNYKNPKL
TRMLTFKFYMPKKATELKHLQCLEEELKHLEEVLNLAQSKNFHLR
PRDLISNINVIVLELKGSETTFMCEYADETATIVEFLNRWITFIQ
SIISTLT
P-0813 knob chain
(SEQ ID NO: 126)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLHDLQMILNGINNYKNPKL
TRMLTFKFYMPKKATELKHLQCLEEELKNLEEVLNLAQSKNFHLR
PRDLISNINVIVLELKGSETTFMCEYADETATIVEFLNRWITFIQ
SIISTLT
P-0814 knob chain
(SEQ ID NO: 127)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLQDLQMILNGINNYKNPKL
TRMLTFKFYMPKKATELKHLQCLEEELKQLEEVLNLAQSKNFHLR
PRDLISNINVIVLELKGSETTFMCEYADETATIVEFLNRWITFIQ
SIISTLT
P-0815 knob chain
(SEQ ID NO: 128)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLQDLQMILNGINNYKNPKL
TRMLTFKFYMPKKATELKHLQCLEEELKHLEEVLNLAQSKNFHLR
PRDLISNINVIVLELKGSETTFMCEYADETATIVEFLNRWITFIQ
SIISTLT
P-0816 knob chain
(SEQ ID NO: 129)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLQDLQMILNGINNYKNPKL
TRMLTFKFYMPKKATELKHLQCLEEELKNLEEVLNLAQSKNFHLR
PRDLISNINVIVLELKGSETTFMCEYADETATIVEFLNRWITFIQ
SIISTLT
P-0732 knob chain
(SEQ ID NO: 130)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIE
SIISTLT
P-0758
(SEQ ID NO: 131)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLHDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0760
(SEQ ID NO: 132)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLQDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
P-0762
(SEQ ID NO: 133)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGSGGGSAPTSSSTKKTQLQLEHLLYDLQMILNGINNYKNPKL
TRMLTFKFYMPKKATELKHLQCLEEELKRLEEVLNLAQSKNFHLR
PRDLISNINVIVLELKGSETTFMCEYADETATIVEFLNRWITFIQ
SIISTLT
Knob-Fc with extended in vivo half-life
(SEQ ID NO: 134)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHAHYTQKSLSLSP
G
Hole-Fc with extended in vivo half-life
(SEQ ID NO: 135)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
EEMTKNQVSLSCAVKGFYPSDIAVEWESNGQPENNYKTTPPVLDS
DGSFFLVSKLTVDKSRWQQGNVFSCSVMHEALHAHYTQKSLSLSP
G
Humanized anti-FAP antibody heavy chain
(SEQ ID NO: 136)
QVQLVQSGAEVKKPGASVKVSCKASGYTFTENIIHWVRQAPGQGL
EWMGWFHPGSGSIKYAQKFQGRVTMTADKSTSTVYMELSSLRSED
TAVYYCARHGGTGRGAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPEAAGAPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPG
Humanized anti-FAP antibody kappa light chain
(SEQ ID NO: 137)
DIQMTQSPSSLSASVGDRVTITCRASRSISTSAYSYMHWYQQKPG
KAPKLLIYLASNLESGVPSRFSGSGSGTDFTLTISSLQPEDFATY
YCQHSRELPYTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Human PD-1 antagonist antibody heavy chain
(SEQ ID NO: 138)
EVQLVQSGAEVKKPGASVKVSCKASGYRFTSYGISWVRQAPGQGL
EWMGWISAYNGNTNYAQKLQGRVTMTTDTSTNTAYMELRSLRSDD
TAVYYCARDADYSSGSGYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPEAAGAPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPG
Human PD-1 antagonist antibody LA
(SEQ ID NO: 139)
SYELTQPPSVSVSPGQTARITCSGDALPKQYAYWYQQKPGQAPVM
VIYKDTERPSGIPERFSGSSSGTKVTLTISGVQAEDEADYYCQSA
DNSITYRVFGGGTKVTVLGQPKAAPSVTLFPPSSEELQANKATLV
CLISDFYPGAVTVAWKADSSPVKAGVETTTPSKQSNNKYAASSYL
SLTPEQWKSHRSYSCQVTHEGSTVEKTVAPTECS
Humanized PD-1 antagonist antibody-HC
(SEQ ID NO: 140)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTIPPVLDSDGSFF
LYSKLTVDKSRWQQGNVFSCSVMHEALHNHYTQKSLSLSPG
Humanized PD-1 antagonist antibody-Lκ
(SEQ ID NO: 141)
DIVMTQSPLSLPVTPGEPASITCKASQDVETVVAWYLQKPGQSPR
LLIYWASTRHTGVPDRFSGSGSGTDFTLKISRVEAEDVGVYYCQQ
YSRYPWTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Humanized PD-1 antagonist antibody-HC
(SEQ ID NO: 142)
QGQLVQSGAEVKKPGASVKVSCKASGYTFTDYEMHWVRQAPGQGL
EWMGVIESETGGTAYNQKFKGRAKITADKSTSTAYMELSSLRSED
TAVYYCTREGITTVATTYYWYFDVWGQGTTVTVSSASTKGPSVFP
LAPCSRSTSESTAALGCLVKDYFPEPVTVSWNSGALTSGVHTFPA
VLQSSGLYSLSSVVTVPSSSLGTKTYTCNVDHKPSNTKVDKRVES
KYGPPCPPCPAPEFLGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
GK
Humanized PD-1 antagonist antibody-Lκ
(SEQ ID NO: 143)
DVVMTQSPLSLPVTLGQPASISCRSSQSIVHSNGNTYLEWYLQKP
GQSPQLLIYKVSNRFSGVPDRFSGSGSGTDFTLKISRVEAEDVGV
YYCFQGSHVPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
Humanized PD-1 antagonist antibody-HC
(SEQ ID NO: 144)
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGK
Humanized PD-1 antagonist antibody-Lκ
(SEQ ID NO: 145)
EIVLTQSPAILSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Human PD-1 antagonist antibody-HC
(SEQ ID NO: 146)
QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSSASTKGPSVFPLAPCSRSTSEST
AALGCLVKDYFPEPVTVSWNSGALTSGVHTFPAVLQSSGLYSLSS
VVTVPSSSLGTKTYTCNVDHKPSNTKVDKRVESKYGPPCPPCPAP
EFLGGPSVFLFPPKPKDTLMISRTPEVTCVVVDVSQEDPEVQFNW
YVDGVEVHNAKTKPREEQFNSTYRVVSVLTVLHQDWLNGKEYKCK
VSNKGLPSSIEKTISKAKGQPREPQVYTLPPSQEEMTKNQVSLTC
LVKGFYPSDIAVEWESNGQPENNYKTTPPVLDSDGSFFLYSRLTV
DKSRWQEGNVFSCSVMHEALHNHYTQKSLSLSLGK
Human PD-1 antagonist antibody-Lκ
SEQ ID NO: 147)
EIVLTQSPATLSLSPGERATLSCRASQSVSSYLAWYQQKPGQAPR
LLIYDASNRATGIPARFSGSGSGTDFTLTISSLEPEDFAVYYCQQ
SSNWPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Humanized PD-L1 antagonist antibody-HC
(SEQ ID NO: 148)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYASTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPGK
Humanized PD-L1 antagonist antibody-Lκ
(SEQ ID NO: 149)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Human CTLA-4 antagonist antibody-HC
(SEQ ID NO: 150)
QVQLVESGGGVVQPGRSLRLSCAASGFTFSSYTMHWVRQAPGKGL
EWVTFISYDGNNKYYADSVKGRFTISRDNSKNTLYLQMNSLRAED
TAIYYCARTGWLGPFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKRVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTIPPVLDSDGSF
FLYSKLTVDKSRWQQGNVFSCSVMHEALHNHYTQKSLSLSPGK
Human CTLA-4 antagonist antibody-Lκ
(SEQ ID NO: 151)
EIVLTQSPGTLSLSPGERATLSCRASQSVGSSYLAWYQQKPGQAP
RLLIYGAFSRATGIPDRFSGSGSGTDFTLTISRLEPEDFAVYYCQ
QYGSSPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC
Human CD40 agonist antibody-HC
(SEQ ID NO: 152)
QVQLVQSGAEVKKPGASVKVSCKASGYTFTGYYMHWVRQAPGQGL
EWMGWINPDSGGTNYAQKFQGRVTMTRDTSISTAYMELNRLRSDD
TAVYYCARDQPLGYCTNGVCSYFDYWGQGTLVTVSSASTKGPSVF
PLAPCSRSTSESTAALGCLVKDYFPEPVTVSWNSGALTSGVHTFP
AVLQSSGLYSLSSVVTVPSSNFGTQTYTCNVDHKPSNTKVDKTVE
RKCCVECPPCPAPPVAGPSVFLFPPKPKDTLMISRTPEVTCVVVD
VSHEDPEVQFNWYVDGVEVHNAKTKPREEQFNSTFRVVSVLTVVH
QDWLNGKEYKCKVSNKGLPAPIEKTISKTKGQPREPQVYTLPPSR
EEMTKNQVSLTCLVKGFYPSDISVEWESNGQPENNYKTTPPMLDS
DGSFFLYSKLTVDKSRWQQGNVFSCSVMHEALHNHYTQKSLSLSP
GK
Human CD40 agonist antibody-Lκ
(SEQ ID NO: 153)
DIQMTQSPSSVSASVGDRVTITCRASQGIYSWLAWYQQKPGKAPN
LLIYTASTLQSGVPSRFSGSGSGTDFTLTISSLQPEDFATYYCQQ
ANIFPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Humanized anti-fibronectin antibody-HC
(SEQ ID NO: 154)
EVQLLESGGGLVQPGGSLRLSCAASGFTFSSFSMSWVRQAPGKGL
EWVSSISGSSGTTYYADSVKGRFTISRDSKNTLYLQMNSLRAEDT
AVYYCAKPFPYFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKRVEPKSCDKTHTCP
PCPAPELLGGPSVFLFPPKPKDTLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSRDELTKNQ
VSLTCLVKGFYPSDIAVEWESNGQPENNYKTTPPVLDSDGSFFLY
SKLTVDKSRWQQGNVFSCSVMHEALHNHYTQKSLSLSPGK
Humanized anti-fibronectin antibody-Lκ
(SEQ ID NO: 155)
EIVLTQSPGTLSLSPGERATLSCRASQSVSSSFLAWYQQKPGQAP
RLLIYYASSRATGIPDRFSGSGSGTDFTLTISRLEPEDFAVYYCQ
QTGRIPPTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC
Chimeric anti-CD20 antibody-HC
(SEQ ID NO: 156)
QVQLQQPGAELVKPGASVKMSCKASGYTFTSYNMHWVKQTPGRGL
EWIGAIYPGNGDTSYNQKFKGKATLTADKSSSTAYMQLSSLTSED
SAVYYCARSTYYGGDWYFNVWGAGTTVTVSAASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKAEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVMHEALHNHYTQKSLSLSPG
K
Chimeric anti-CD20 antibody-Lκ
(SEQ ID NO: 157)
QIVLSQSPAILSASPGEKVTMTCRASSSVSYIHWFQQKPGSSPKP
WIYATSNLASGVPVRFSGSGSGTSYSLTISRVEAEDAATYYCQQW
TSNPPTFGGGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
Humanized anti-Her2 antibody-HC
(SEQ ID NO: 158)
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK
Humanized anti-Her2 antibody-Lκ
(SEQ ID NO: 159)
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Chimeric anti-EGFR antibody-HC
(SEQ ID NO: 160)
QVQLKQSGPGLVQPSQSLSITCTVSGFSLTNYGVHWVRQSPGKGL
EWLGVIWSGGNTDYNTPFTSRLSINKDNSKSQVFFKMNSLQSNDT
AIYYCARALTYYDYEFAYWGQGTLVTVSAASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTIPPVLDSDGS
FFLYSKLTVDKSRWQQGNVFSCSVMHEALHNHYTQKSLSLSPGK
Chimeric anti-EGFR antibody-Lκ
(SEQ ID NO: 161)
DILLTQSPVILSVSPGERVSFSCRASQSIGTNIHWYQQRTNGSPR
LLIKYASESISGIPSRFSGSGSGTDFTLSINSVESEDIADYYCQQ
NNNWPTTFGAGTKLELKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Human IgG1 CH1-CH2-CH3 domain with
reduced/abolished effector function
(SEQ ID NO: 162)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGAPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG
Human IgG1 CH1-CH2-CH3 domain with
reduced/abolished effector function
knob chain
(SEQ ID NO: 163)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGAPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVCTLPPSREEMTKNQVSLWCLVKGFYPSDIAVEWESNGQP
ENNYKTTPPVLDSDGSFFLYSKLTVDKSRWQQGNVFSCSVMHEAL
HNHYTQKSLSLSPG
Human IgG1 CH1-CH2-CH3 domain with
reduced/abolished effector function
Hole chain
(SEQ ID NO: 164)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGAPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPCREEMTKNQVSLSCAVKGFYPSDIAVEWESNGQP
ENNYKTTPPVLDSDGSFFLVSKLTVDKSRWQQGNVFSCSVMHEAL
HNHYTQKSLSLSPG
Humanized PD-1 antagonist antibody-HC-
IL-2 variant
(SEQ ID NO: 165)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTIPPVLDSDGSFF
LYSKLTVDKSRWQQGNVFSCSVMHEALHAHYTQKSLSLSPGGGGS
GGGSAPTSSSTKKTQLQLEHLLHDLQMILNGINNYKNPKLTRMLT
FKFYMPKKATELKHLQCLEEELKRLEEVLNLAQSKNFHLRPRDLI
SNINVIVLELKGSETTFMCEYADETATIVEFLNRWITFIQSIIST
LT
Humanized PD-1 antagonist antibody-HC-
IL-2 variant knob chain
(SEQ ID NO: 166)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVCTLPPSREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGS
GGGSAPTSSSTKKTQLQLEHLLLDLQMILNGINNYKNPKLTRMLT
FKFYMPKKATELKHLQCLEEELKRLEEVLNLAQSKNFHLRPRDLI
SNINVIVLELKGSETTFMCEYADETATIVEFLNRWITFIQSIIST
LT
Humanized PD-1 antagonist antibody-HC-
IL-2 variant knob chain
(SEQ ID NO: 167)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVCTLPPSREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGS
GGGSAPTSSSTKKTQLQLEHLLLDLQMILNGINNYKNPKLTRMLT
FKFYMPKKATELKHLQCLEEELKQLEEVLNLAQSKNFHLRPRDLI
SNINVIVLELKGSETTFMCEYADETATIVEFLNRWITFIQSIIST
LT
Humanized PD-1 antagonist antibody-HC-
IL-2 variant knob chain
(SEQ ID NO: 168)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVCTLPPSREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGS
GGGSAPTSSSTKKTQLQLEHLLQDLQMILNGINNYKNPKLTRMLT
FKFYMPKKATELKHLQCLEEELKQLEEVLNLAQSKNFHLRPRDLI
SNINVIVLELKGSETTFMCEYADETATIVEFLNRWITFIQSIIST
LT
Humanized PD-1 antagonist antibody-
IgG1-HC hole chain
(SEQ ID NO: 169)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLSCAVKGFYPSDIAVEWESNGQPENNYKTTPPVLDSDGSFF
LVSKLTVDKSRWQQGNVFSCSVMHEALHNHYTQKSLSLSPG
Human
IL-2Rα Sushi domains sequence
(SEQ ID NO: 170)
ELCDDDPPEIPHATFKAMAYKEGTMLNCECKRGFRRIKSGSLYML
CTGNSSHSSWDNQCQCTSSATRNTTKQVTPQPEEQKERKTTEMQS
PMQPVDQASLPGHCREPPPWENEATERIYHFVVGQMVYYQCVQGY
RALHRGPAESVCKMTHGKTRWTQPQLICTG
P-0327
(SEQ ID NO: 171)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GGGGGSGGGGSGGGGSELCDDDPPEIPHATFKAMAYKEGTMLNCE
CKRGFRRIKSGSLYMLCTGNSSHSSWDNQCQCTSSATRNTTKQVT
PQPEEQKERKTTEMQSPMQPVDQASLPGHCREPPPWENEATERIY
HFVVGQMVYYQCVQGYRALHRGPAESVCKMTHGKTRWTQPQLICT
GGGGGSGGGGSGGGGSAPTSSSTKKTQLQLEHLLLDLQMILNGIN
NYKNPKLTRMLTFKFYMPKKATELKHLQCLEEELKPLEEVLNLAQ
SKNFHLRPRDLISNINVIVLELKGSETTFMCEYADETATIVEFLN
RWITFSQSIISTLT
P-0422
(SEQ ID NO: 172)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFSQSIISTLTGG
GGSGGGGSGGGGSELCDDDPPEIPHATFKAMAYKEGTMLNCECKR
GFRRIKSGSLYMLCTGNSSHSSWDNQCQCTSSATRNTTKQVTPQP
EEQKERKTTEMQSPMQPVDQASLPGHCREPPPWENEATERIYHFV
VGQMVYYQCVQGYRALHRGPAESVCKMTHGKTRWTQPQLICTGGG
GSGGGGSGGGGSCPPCPAPEAAGAPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVMHEALHNHYTQ
KSLSLSPG
P-0482-Hole chain
(SEQ ID NO: 173)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
EEMTKNQVSLSCAVKGFYPSDIAVEWESNGQPENNYKTTPPVLDS
DGSFFLVSKLTVDKSRWQQGNVFSCSVMHEALHNHYTQKSLSLSP
GGGGSGGGSAPTSSSTKKTQLQLEHLLLDLQMILNGINNYKNPKL
TRMLTFKFYMPKKATELKHLQCLEEELKPLEEVLNLAQSKNFHLR
PRDLISNINVIVLELKGSETTFMCEYADETATIVEFLNRWITFSQ
SIISTLT
P-0482-Knob chain
(SEQ ID NO: 174)
DKTHTCPPCPAPEAAGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGGSGGGGSGGGGSELCDDDPPEIPHATFKAMAYKEGTMLNCE
CKRGFRRIKSGSLYMLCTGNSSHSSWDNQCQCTSSATRNTTKQVT
PQPEEQKERKTTEMQSPMQPVDQASLPGHCREPPPWENEATERIY
HFVVGQMVYYQCVQGYRALHRGPAESVCKMTHGKTRWTQPQLICT
Humanized PD-1 antagonist antibody-IgG1-
HC hole chain
(SEQ ID NO: 175)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVCTLPPSREEMTK
NQVSLSCAVKGFYPSDIAVEWESNGQPENNYKTTPPVLDSDGSFF
LVSKLTVDKSRWQQGNVFSCSVMHEALHNHYTQKSLSLSPG
Humanized PD-1 antagonist antibody-HC-
IL-2 variant knob chain
(SEQ ID NO: 176)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLHDLQMILNGINNYKNP
KLTRMLTFKFYMPKKATELKHLQCLEEELKRLEEVLNLAQSKNFH
LRPRDLISNINVIVLELKGSETTFMCEYADETATIVEFLNRWITF
IQSIISTLT
Humanized PD-1 antagonist antibody-HC-
IL-2 variant knob chain
(SEQ ID NO: 177)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLLDLQMILNGINNYKNP
KLTRMLTFKFYMPKKATELKHLQCLEEELKRLEEVLNLAQSKNFH
LRPRDLISNINVIVLELKGSETTFMCEYADETATIVEFLNRWITF
IQSIISTLT
Humanized PD-1 antagonist antibody-HC-
IL-2 variant knob chain
(SEQ ID NO: 178)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLQDLQMILNGINNYKNP
KLTRMLTFKFYMPKKATELKHLQCLEEELKQLEEVLNLAQSKNFH
LRPRDLISNINVIVLELKGSETTFMCEYADETATIVEFLNRWITF
IQSIISTLT
Humanized PD-1 antagonist antibody-HC-
IL-2 variant knob chain
(SEQ ID NO: 179)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLQDLQMILNGINNYKNP
KLTRMLTFKFYMPKKATELKHLQCLEEELKRLEEVLNLAQSKNFH
LRPRDLISNINVIVLELKGSETTFMCEYADETATIVEFLNRWITF
IQSIISTLT
Humanized PD-1 antagonist antibody-HC-
IL-2 variant knob chain
(SEQ ID NO: 180)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLHDLQMILNGINNYKNP
KLTRMLTFKFYMPKKATELKHLQCLEEELKQLEEVLNLAQSKNFH
LRPRDLISNINVIVLELKGSETTFMCEYADETATIVEFLNRWITF
IQSIISTLT
Humanized PD-1 antagonist antibody-HC-
IL-2 variant knob chain
(SEQ ID NO: 181)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSAPTSSSTKKTQLQLEHLLQDLQMILNGINNYKNP
KLTRMLTFKFYMPKKATELKHLQCLEEELKNLEEVLNLAQSKNFH
LRPRDLISNINVIVLELKGSETTFMCEYADETATIVEFLNRWITF
IQSIISTLT
Benchmark PD-1 antagonist antibody-HC-
hole chain
(SEQ ID NO: 182)
EVQLLESGGGLVQPGGSLRLSCAASGFSFSSYTMSWVRQAPGKGL
EWVATISGGGRDIYYPDSVKGRFTISRDNSKNTLYLQMNSLRAED
TAVYYCVLLTGRVYFALDSWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPEAAGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALGAPIEKTISKAKGQPREPQVCTLPPSRDE
LTKNQVSLSCAVKGFYPSDIAVEWESNGQPENNYKTTPPVLDSDG
SFFLVSKLTVDKSRWQQGNVFSCSVMHEALHNHYTQKSLSLSPG
Benchmark PD-1 antagonist antibody-HC-
Benchmark IL-2 variant knob chain
(SEQ ID NO: 183)
EVQLLESGGGLVQPGGSLRLSCAASGFSFSSYTMSWVRQAPGKGL
EWVATISGGGRDIYYPDSVKGRFTISRDNSKNTLYLQMNSLRAED
TAVYYCVLLTGRVYFALDSWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPEAAGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALGAPIEKTISKAKGQPREPQVYTLPPCRDE
LTKNQVSLWCLVKGFYPSDIAVEWESNGQPENNYKTTPPVLDSDG
SFFLYSKLTVDKSRWQQGNVFSCSVMHEALHNHYTQKSLSLSPGG
GGGSGGGGSGGGGSAPASSSTKKTQLQLEHLLLDLQMILNGINNY
KNPKLTRMLTAKFAMPKKATELKHLQCLEEELKPLEEVLNGAQSK
NFHLRPRDLISNINVIVLELKGSETTFMCEYADETATIVEFLNRW
ITFAQSIISTLT
Benchmark PD-1 antagonist antibody-LA
(SEQ ID NO: 184)
EVQLLESGGGLVQPGGSLRLSCAASGFSFSSYTMSWVRQAPGKGL
EWVATISGGGRDIYYPDSVKGRFTISRDNSKNTLYLQMNSLRAED
TAVYYCVLLTGRVYFALDSWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPEAAGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALGAPIEKTISKAKGQPREPQVCTLPPSRDE
LTKNQVSLSCAVKGFYPSDIAVEWESNGQPENNYKTTPPVLDSDG
SFFLVSKLTVDKSRWQQGNVFSCSVMHEALHNHYTQKSLSLSPG
Surrogate mouse PD-1 antagonist antibody
HC chain 1
(SEQ ID NO: 185)
EVQLQESGPGLVKPSQSLSLTCSVTGYSITSSYRWNWIRKFPGNR
LEWMGYINSAGISNYNPSLKRRISITRDTSKNQFFLQVNSVTTED
AATYYCARSDNMGTTPFTYWGQGTLVTVSSAKTTPPSVYPLAPGS
AAQTNSMVTLGCLVKGYFPEPVTVTWNSGSLSSGVHTFPAVLQSD
LYTLSSSVTVPSSTWPSQTVTCNVAHPASSTKVDKKIVPRDCGCK
PCICTVPEVSSVFIFPPKPKDVLTITLTPKVTCVVVAISKDDPEV
QFSWFVDDVEVHTAQTKPREEQINSTFRSVSELPIMHQDWLNGKE
FKCRVNSAAFGAPIEKTISKTKGRPKAPQVYTIPPPKKQMAKDKV
SLTCMITNFFPEDITVEWQWNGQPAENYKNTQPIMKTDGSYFVYS
KLNVQKSNWEAGNTFTCSVLHEGLHNHHTEKSLSHSPG
Surrogate mouse PD-1 antagonist antibody
HC chain 2
(SEQ ID NO: 186)
EVQLQESGPGLVKPSQSLSLTCSVTGYSITSSYRWNWIRKFPGNR
LEWMGYINSAGISNYNPSLKRRISITRDTSKNQFFLQVNSVTTED
AATYYCARSDNMGTTPFTYWGQGTLVTVSSAKTTPPSVYPLAPGS
AAQTNSMVTLGCLVKGYFPEPVTVTWNSGSLSSGVHTFPAVLQSD
LYTLSSSVTVPSSTWPSQTVTCNVAHPASSTKVDKKIVPRDCGCK
PCICTVPEVSSVFIFPPKPKDVLTITLTPKVTCVVVAISKDDPEV
QFSWFVDDVEVHTAQTKPREEQINSTFRSVSELPIMHQDWLNGKE
FKCRVNSAAFGAPIEKTISKTKGRPKAPQVYTIPPPKEQMAKDKV
SLTCMITNFFPEDITVEWQWNGQPAENYDNTQPIMDTDGSYFVYS
DLNVQKSNWEAGNTFTCSVLHEGLHNHHTEKSLSHSPG
Surrogate mouse PD-1 antagonist antibody LC
(SEQ ID NO: 187)
DIVMTQGTLPNPVPSGESVSITCRSSKSLLYSDGKTYLNWYLQRP
GQSPQLLIYWMSTRASGVSDRFSGSGSGTDFTLKISGVEAEDVGI
YYCQQGLEFPTFGGGTKLELKRTDAAPTVSIFPPSSEQLTSGGAS
VVCFLNNFYPRDINVKWKIDGSERQNGVLNSWTDQDSKDSTYSMS
STLTLTKDEYERHNSYTCEATHKTSTSPIVKSFNRNEC
Benchmark IL-2 variant
(SEQ ID NO: 188)
APASSSTKKTQLQLEHLLLDLQMILNGINNYKNPKLTRMLTAKFA
MPKKATELKHLQCLEEELKPLEEVLNGAQSKNFHLRPRDLISNIN
VIVLELKGSETTFMCEYADETATIVEFLNRWITFAQSIISTLT
Humanized PD-1 antagonist antibody-HC-
IL-2 variant knob chain
(SEQ ID NO: 189)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSKTQLQLEHLLLDLQMILNGINNYKNPKLTRMLTF
KFYMPKKATELKHLQCLEEELKRLEEVLNLAQSKNFHLRPRDLIS
NINVIVLELKGSETTFMCEYADETATIVEFLNRWITFIQSIISTL
T
Humanized PD-1 antagonist antibody-HC-
IL-2 variant knob chain
(SEQ ID NO: 190)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSTQLQLEHLLLDLQMILNGINNYKNPKLTRMLTFK
FYMPKKATELKHLQCLEEELKRLEEVLNLAQSKNFHLRPRDLISN
INVIVLELKGSETTFMCEYADETATIVEFLNRWITFIQSIISTLT
Humanized PD-1 antagonist antibody-HC-IL-2
variant knob chain
(SEQ ID NO: 191)
EVQLVESGGGLVKPGGSLRLSCAASGFTFSSYDMSWVRQAPGKGL
EWVATISGGGSYTYYPDSVKGRFTISRDNAKNSLYLQMNSLRAED
TAVYYCASPDSSGVAYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGAPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPCREEMTK
NQVSLWCLVKGFYPSDIAVEWESNGQPENNYKTTPPVLDSDGSFF
LYSKLTVDKSRWQQGNVFSCSVMHEALHNHYTQKSLSLSPGGGGG
SGGGGSGGGGSQLQLEHLLLDLQMILNGINNYKNPKLTRMLTFKF
YMPKKATELKHLQCLEEELKRLEEVLNLAQSKNFHLRPRDLISNI
NVIVLELKGSETTFMCEYADETATIVEFLNRWITFIQSIISTLT

1-39. (canceled)

40. An isolated IL-2 variant polypeptide, wherein said IL-2 variant polypeptide comprises the amino acid sequence of SEQ ID NO: 3 having one or more of amino acid residues position R38, T41, F42, F44, E62, P65, E68, Y107, or S125 substituted with another amino acid, and wherein said IL-2 variant polypeptide demonstrates reduced binding to IL-2Rα with lower Treg activity as compared to the polypeptide represented by SEQ ID NO: 3, yet retains the ability to bind to and activate the IL-2Rβγ complex.

41. The IL-2 variant polypeptide according to claim 40, further comprising an amino acid substitution at position 126.

42. The IL-2 variant polypeptide according to claim 41, wherein the amino acid substitution is selected from the group consisting of: the substitution of L19D, L19H, L19N, L19P, L19Q, L19R, L19S, L19Y at position 19, the substitution of R38A, R38F, R38G at position 38, the substitution of T41A, T41G, T41V at position 41, the substitution of F42A at position 42, the substitution of F44G, F44V at position 44, the substitution of E62A, E62F, E62H, E62L at position 62, the substitution of P65A, P65E, P65G, P65H, P65K, P65N, P65Q, P65R at position 65, the substitution of E68E, E68F, E68H, E68L, E68P at position 68, the substitution of Y107G, Y107H, Y107L, Y107V at position 107, the substitution of S125I at position 125, and the substitution of Q126E, Q126K at position 126.

43. The IL-2 variant polypeptide according to claim 40, wherein the IL-2 variant polypeptide comprises two amino acid substitutions at amino acid residues position P65 and S125 of SEQ ID NO: 3.

44. The IL-2 variant polypeptide according to claim 40, wherein the IL-2 variant polypeptide comprises three amino acid substitutions at amino acid residues position L19, P65 and S125 of SEQ ID NO: 3.

45. The IL-2 variant polypeptide according to claim 41, wherein the IL-2 variant polypeptide comprises four amino acid substitutions at amino acid residues position L19, P65, S125 and Q126 of SEQ ID NO: 3.

46. The IL-2 variant polypeptide according to claim 41, wherein the IL-2 variant polypeptide comprises the amino acid sequence is selected from the group consisting of the amino acid sequences set forth in SEQ ID NOS: 31-66 and SEQ ID NOS: 111-120.

47. An isolated fusion protein comprising 1) an IL-2 variant polypeptide according to claims 41 and 2) a heterologous protein selected from an isolated fusion protein wherein said IL-2 variant polypeptide is fused at its N-terminal amino acid to the C-terminal amino acid of the heterologous protein and an isolated fusion protein wherein said IL-2 variant polypeptide is fused at its C-terminal amino acid to the N-terminal amino acid of the heterologous protein; optionally through a peptide linker, either in a monomeric or a dimeric form.

48. The isolated fusion protein of claim 47, wherein said IL-2 variant polypeptide is fused to said heterologous protein through a peptide linker, either in dimeric or monomeric form.

49. The fusion protein of claim 48, wherein said peptide linker comprises between 1 and 40 amino acids.

50. The isolated fusion protein according to claim 47, wherein the heterologous protein is a targeting moiety in the form of an antibody, an antibody heavy chain or light chain, an antibody fragment, a protein and a peptide which targets a tumor associated antigen (TAA).

51. The isolated fusion protein according to claim 50, wherein the antibody, or an antibody fragment is selected from the group consisting of: PD-1 antagonistic antibodies; PD-L1 antagonistic antibodies; TIGIT antagonistic antibodies; CTLA-4 antagonistic antibodies; CD20 antagonistic antibodies; Her-2/neu antagonistic antibodies; EGFR antagonistic antibodies; Fibroblast Activation Protein (FAP) antagonistic antibodies; anti-inflammatory antibodies against integrin α₄β₇; TNFα antagonistic antibodies; and agonistic CD40 antibodies.

52. The isolated fusion protein according to claim 50, wherein the heterologous protein is an antibody or an antibody fragment to an immune checkpoint modulator.

53. The isolated fusion protein according to claim 52, wherein the antibody is an antagonistic Programmed Death-1 (PD-1) antibody or antibody fragment.

54. The isolated fusion protein according to claim 53, wherein the antibody is an antagonistic humanized PD-1 antibody selected from the antibody comprising the heavy chain and light chain amino acid sequences set forth in SEQ ID NOS: 138 and 139; the heavy chain and light chain amino acid sequences set forth in SEQ ID NOS: 140 and 141; the heavy chain and light chain amino acid sequences set forth in SEQ ID NOS: 142 and 143; the heavy chain and light chain amino acid sequences set forth in SEQ ID NOS: 144 and 145; and the heavy chain and light chain amino acid sequences set forth in SEQ ID NOS: 146 and 147.

55. A pharmaceutical composition selected from the group consisting of a pharmaceutical composition comprising an IL-2 variant polypeptide according to claim 40 in admixture with a pharmaceutically acceptable carrier, and an isolated fusion protein according to claim 47 in admixture with a pharmaceutically acceptable carrier.

56. A method of treating cancer in a subject, comprising administering to the subject a therapeutically effective amount of the pharmaceutical composition according to claim 55.

57. The method according to claim 56, wherein the method further comprises administering a second therapeutic agent or modality capable of treating cancer in a subject.

58. A host cell comprising a nucleic acid molecule encoding an IL-2 variant polypeptide according to claim 40.

59. A method of producing an IL-2 variant polypeptide according to claim 40 comprising culturing the host cell of claim 58 under conditions promoting the expression of the IL-2 variant polypeptide and recovering the IL-2 variant polypeptide.
